(19) United States
(12) Patent Application Publication
(10) Pub. No.: US 2023/0112908 A1
(43) Pub. Date: Apr. 13, 2023

(54) MODIFIABLE PHARMACEUTICAL PROTOCOL FOR MIGRAINE TREATMENT AND ASSESSMENT OF DRUG EFFICACY THEREOF

(71) Applicant: Twill, Inc., New York, NY (US)

(72) Inventors: Jared Minkel, Kingstown, RI (US); Janis Martman, British Columbia (CA); Chris Wasden, Park City, UT (US); Ran Zilca, Ra'anana (IL); Tomer Ben-Kiki, New York, NY (US)

(73) Assignee: Twill, Inc., New York, NY (US)

(21) Appl. No.: 17/964,869

(22) Filed: Oct. 12, 2022

Related U.S. Application Data
(60) Provisional application No. 63/254,623, filed on Oct. 12, 2021.

(51) Int. Cl. G16H 20/10

(52) U.S. Cl. CPC G16H 20/10

(57) ABSTRACT

The present disclosure relates to a treatment protocol for treatment of migraine comprising an abortive drug adapted to bind with a plurality of calcitonin gene-related peptides (CGRP) receptors. The abortive drug may be adapted to mitigate vasodilation in a user. The treatment protocol may comprise evaluating medication-relevant information from a user input to determine positive and/or negative effects of the abortive drug regimen. The treatment protocol may include a preventative drug regimen and a corresponding preventative drug. The treatment protocol may be configured to evaluate the efficacy of the abortive and/or preventive drug regimens based on the determined positive and/or negative effects. In a further embodiment, the drug protocol may be configured to generate suggested alterations to either regimen based on the efficacy evaluation.

### FIELD OF THE INVENTION

The present invention relates to a treatment protocol utilizing drugs adapted to bind with a plurality of calcitonin gene-related peptides (CGRP) receptors to treat migraine. Specifically, the present invention relates to a modifiable drug protocol utilizing abortive drugs and/or preventative drugs, wherein the regimen of each is modifiable as a function of a user’s positive and/or negative side effects.

### INTRODUCTION

Migraine is a complex, common neurological condition characterized by severe, episodic attacks of headache and associated features such as nausea, vomiting, sensitivity to light, sound or movement. Moreover, in order to properly treat, prevent, and/or abort a migraine headache a patient must adhere and comply with the treatment as best suited for that particular patient.

In some patients, the headache is preceded or accompanied by an aura. The headache pain may be severe and sometimes occurs on one side of the brain. This is called unilateral migraine. Migraines in about 15% of patients are “side-locked” in that they only get migraine headaches on one side. In North America and Western Europe, the overall prevalence of migraine patients is 11% of the general population, i.e., 6% in males and 15-18% in females. The median frequency of migraine attacks in individuals is one or two per month, though the deviation from this mean is substantial. There is a strong genetic component to migraines.

A chronic migraine is when a migraine occurs 15 or more days per month. Symptoms in chronic migraine often change frequently as may the severity of the pain. Primarily due to the high frequency of chronic migraine, it has a particularly debilitating impact on the patient’s quality of life and has the potential to be a primary feature of the patient’s life. Sufferers of chronic migraine have a high incidence of depression, anxiety, employment issues and lower socioeconomic status than the general public. Chronic migraine affects about 2% of the general population.

A migraine is much more than a bad headache. Migraine attacks are often disruptive to daily life. The throbbing pain is often debilitating and its debilitating impact typically lasts several hours but may last days. Onset of a migraine attack may be associated with triggers that include movement, light, sound and many others. A migraine may involve one or more symptoms like neurological pain, tiredness, nausea, visual disturbances, numbness and tingling, irritability, difficulty speaking, temporary loss of vision and many more. Migraine is a common neurological disease having a most prevalent symptom of a throbbing, pulsing headache on one side of head. Migraine symptoms typically worsen with physical activity, lights, sounds or smells.

It would be desirable to provide a treatment protocol comprising a pharmaceutical configured to prevent vasodilation in order to mitigate negative effects of migraine. It would be further desirable to provide a treatment protocol comprising a means of tracking the patient’s adherence. It would be yet further desirable to provide a treatment protocol adapted to adjust the regimens according to the patient’s adherence and/or treatment performance.

### SUMMARY OF AN EMBODIMENT

Disclosed herein are devices and methods involving digital therapeutics for treating migraine, symptoms associated with migraine, symptoms associated with side-effects of migraine medications, and comorbidities of migraine. A digital device for use in such treatment includes a display, an input device, one or more processors, networking interfaces and memory storing one or more software programs configured to be executed by the one or more processors.

The invention of the present disclosure may be a treatment protocol comprising an abortive drug configured to inhibit at least one of a plurality of a calcitonin superfamily of peptides, the abortive drug adapted to bind with a plurality of calcitonin gene-related peptides (CGRP) receptors, the abortive drug further comprising an abortive dosage, wherein the plurality of the calcitonin superfamily of peptides comprises one or more CGRPs, wherein the one or more CGRPs comprise a 37 amino acid vasoactive neuropeptide, and wherein the abortive drug may bind to the plurality of CGRP receptors to mitigate vasodilation. In a further embodiment, the treatment protocol includes a client device comprising at least one processor, at least one memory comprising a drug database and computer-executable instructions which, when executed by the at least one processor, cause the client device to: receive, via the at least one memory, an initial abortive drug regimen, wherein the abortive drug regimen comprises one or more abortive drugs and one or more abortive dosages; and receive, via the client device, a user input through an interactive session, wherein the interactive session is configured for bidirectional communication between the client device and a user.

In an aspect, the computer-executable instructions may further cause the client device to: extract, via the at least one processor, medication-relevant information from the user input, wherein the medication-relevant information is correlated to drug-specific information stored within the drug database; and determine, via the at least one processor, based on the medication-relevant information and the user input, a migraine severity of the user. In a further aspect, the computer-executable instructions may further cause the client device to: display, via the client device, an alert configured to inform the user to administer the one or more abortive dosages if the migraine severity is greater than an abortive drug threshold; and analyze, via the at least one processor, the medication-relevant information in view of the drug-specific information to determine whether the initial abortive drug regimen is being adhered to and whether the medication-relevant information comprises one or more side effects or one or more positive effects, wherein the one or more side effects and the one or more positive effects are correlated to the one or more abortive drugs via the drug database.

In a further embodiment, the treatment protocol further comprises a preventative drug configured to inhibit at least one of the plurality of the calcitonin superfamily of peptides, the preventative drug adapted to bind with the plurality of CGRP receptors, the preventative drug further comprising a preventative dosage, wherein the preventative drug may bind to the plurality of CGRP receptors to mitigate vasodilation. In such an embodiment, the treatment protocol further includes the computer-executable instructions which, when executed by the at least one processor, further cause the client device to: receive, via the at least one memory, an initial preventative drug regimen, wherein the preventative drug regimen comprises one or more preventative drugs and one or more preventative dosages; and display, via the client device, a reminder configured to inform the user to administer the one or more preventative dosages according to the initial preventative drug regimen.

In yet another aspect, the computer-executable instructions further cause the client device to: analyze, via the at least one processor, the medication-relevant information in view of the drug-specific information to determine whether the initial preventative drug regimen is being adhered to and whether the medication-relevant information comprises one or more side effects or one or more positive effects, wherein the one or more side effects and the one or more positive effects are correlated to the one or more preventative drugs via the drug database.

In an embodiment, the computer-executable instructions which, when executed by the at least one processor, further cause the client device to: generate, via the at least one processor, an activity within the interactive session, the activity configured to increase the one or more positive effects and decrease the one or more side effects, wherein the activity is one of a plurality of activity types, and wherein the activity comprises an activity content. In such an embodiment, the computer-executable instructions further cause the processor to receive, via the at least one memory, an activity response; and analyze, via the at least one processor, the activity response to determine whether the one or more side effects and the one or more positive effects have fluctuated relative to the previously assessed one or more side effects and one or more positive effects.

In yet a further embodiment, the computer-executable instructions which, when executed by the at least one processor, further cause the client device to: determine, via the at least one processor, an activity content efficacy and an activity type efficacy; and generate, via the at least one processor, a succeeding activity, wherein the succeeding activity is one of the plurality of activity types according to the activity type efficacy, and wherein the succeeding activity comprises the activity content according to the activity content efficacy.

The treatment protocol may further comprise one or more sensors in electrical communication with the client device, the one or more sensors configured to capture the user input and the activity response, and transmit said user input and said activity response to the at least one memory. The computer-executable instructions which, when executed by the at least one processor, may cause the client device to: generate, via the at least one processor, an updated abortive drug regimen based on the initial abortive drug regimen, the one or more side effects, and the one or more positive effects. Further, the computer-executable instructions which, when executed by the at least one processor, may cause the client device to: generate, via the at least one processor, an updated preventative drug regimen based on the initial preventative drug regimen, the one or more side effects, and the one or more positive effects.

Additional aspects related to this disclosure are set forth, in part, in the description which follows, and, in part, will be obvious from the description, or may be learned by practice of this disclosure.

It is to be understood that both the forgoing and the following descriptions are exemplary and explanatory only and are not intended to limit the claimed disclosure or application thereof in any manner whatsoever.

### BRIEF DESCRIPTION OF THE FIGURES

The present disclosure will become more fully understood from the detailed description and the accompanying drawings, wherein:

FIG. 1 shows an example of a client-server based distributed communication system that can be used to implement an online service for enhancing mental health of users and a dialogue management system for the online service;

FIG. 2 shows an example of a client device of the distributed communication system of FIG. 1;

FIG. 3 shows an example of a block diagram of a server of the distributed communication system of FIG. 1;

FIG. 4 shows an example of a block diagram of the online service;

FIG. 5A shows an example of a block diagram of the dialogue management system;

FIG. 5B shows an example of a dialogue box (also called a dialog box) including a dialogue between the online service of FIG. 4 and a user of the online service using the dialogue management system of FIG. 5A;

FIG. 6 shows an example of a flowchart of a method of conducting a dialogue between the online service of FIG. 4 and a user of the online service using the dialogue management system of FIG. 5A;

FIG. 7 shows an example of a flowchart of a method of creating a master dialogue file for conducting a dialogue between the online service of FIG. 4 and a user of the online service using the dialogue management system of FIG. 5A;

FIG. 8 shows an example of a flowchart of a method of creating a skeleton file for conducting a dialogue between the online service of FIG. 4 and a user of the online service using the dialogue management system of FIG. 5A;

FIG. 9 shows an example of a flowchart of a method of creating a skin file for conducting a dialogue between the online service of FIG. 4 and a user of the online service using the dialogue management system of FIG. 5A;

FIGS. 10A-10N show a table including examples of tracks and activities offered by the online service to the users of the online service of FIG. 4 for improving mental health of the users;

FIGS. 11A-11C show a table including an example of a track, activities of the track, and tasks of the activities offered by the online service for FIG. 4 for improving mental health of the users; and

FIG. 12 is an exemplary flowchart explaining a proactive triaging feature of the present invention.

In the drawings, reference numbers may be reused to identify similar and/or identical elements.

### DETAILED DESCRIPTION

Some aspects and embodiments of the disclosed invention will be described more fully with reference to the accompanying drawings. This disclosed invention may be embodied in many different forms and should not be construed as limited to the aspects and embodiments set forth herein.

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized and that structural changes and/or substitutions of various elements may be made without departing from the scope and spirit of this disclosure. The following detailed description is, therefore, not to be construed in a limited sense.

It is noted that description herein is not intended as an extensive overview, and as such, concepts may be simplified in the interests of clarity and brevity.

All documents mentioned in this application are hereby incorporated by reference in their entirety. Any process described in this application may be performed in any order and may omit any of the steps in the process. Processes may also be combined with other processes or steps of other processes.

An aura is a group of sensory, motor and speech symptoms that usually act like warning signals that a migraine headache is approaching. Sometimes misconstrued as a seizure or stroke, aura typically happens before the headache pain, but can sometimes appear during or even after the migraine episode. An aura can last from 10 to 60 minutes and occur in about 15% to 20% of people who experience migraines.

Aura symptoms include seeing bright flashing dots, sparkles, or lights, blind spots in vision, numb or tingling skin, speech changes, ringing in ears (tinnitus), temporary vision loss, seeing wavy or jagged lines, changes in smell or taste, and a “funny” feeling.

There are several types of migraines and the same type may go by different names. Migraine with aura is also referred to as a complicated migraine, occurring in about 15% to 20% of people with migraine headaches. Migraine without aura is also referred to as common migraine. This type of migraine headache strikes without the warning, though the symptoms are the same, other than lack of aura symptoms. Migraine without head pain is referred to as silent migraine or acephalgic migraine and includes the aura symptom but not the headache that typically follows. Hemiplegic migraine involves temporary paralysis (hemiplegia) or neurological or sensory changes on one side of the body. Onset of hemiplegic migraine headache may be associated with temporary numbness, extreme weakness on one side of the body, a tingling sensation, a loss of sensation and dizziness or vision changes. Sometimes it includes headache and sometimes it does not. Retinal migraine is sometime referred to as ocular migraine and has symptoms including temporary, partial or complete loss of vision in one eye, along with a dull ache behind that eye that may spread. Vision loss may last a minute or as long as months.

Migraine with brainstem aura is migraine accompanied by vertigo, slurred speech, double vision or loss of balance, which symptoms occur before the headache. The headache pain may affect the back of the head. Migraine with brainstem aura symptoms usually occur suddenly and can be associated with the inability to speak properly, ringing in the ears and vomiting. Status migrainosus is a rare and severe type of migraine that can last longer than 72 hours. The headache pain and nausea can be extremely bad. Certain medications, or medication withdrawal, can cause this type of migraine.

The four stages of typical migraine are, in chronological order, the prodrome (premonitory), aura, headache and postdrome. About 30% of people experience symptoms before their headache starts. Prodrome may last a few hours or a few days and is sometimes referred to as the “preheadache” or “premonitory” phase. The aura phase can last as long as 60 minutes or as little as five. Most people do not experience an aura, and some have both the aura and the headache at the same time. Headache lasts about 4 hours to 72 hours. Although sometimes mild, the headache pain is typically intense, starting on one side of the head and spreading to the other side. Postdrome follows the headache and lasts for a day or two. It has been called a migraine “hangover” and 80% of those who have migraines experience it.

The cause(s) of migraine remain little understood. Changes is the brainstem and the interaction of the brainstem with the trigeminal nerve, a major pain pathway, may be involved. Imbalances in brain chemicals, e.g., serotonin, may also be a factor. Serotonin helps regulate pain in the nervous system and its role has been a focus of migraine research. Other neurotransmitters have been receiving attention with regard to migraine research, including calcitonin gene related peptide (CGRP), discussed further hereinbelow.

The primary risk factors for migraine include genetics, gender, stress level and smoking. About 80% of people who get migraine headaches have a first-degree relative with the disease. Migraine headaches are two to three times more prevalent in women than in men, this is especially true for women between the ages of 15 and 55. These two facts, among others, contribute to the strong evidence that female hormones influence risk factors.

Hormonal changes, stress and smoking exist on the border between causes/risk factors for migraine and migraine triggers. Fluctuations in estrogen seem to trigger migraines, as do higher stress levels, smoking, caffeine, sensory stimuli such as strong lights, loud sounds and strong smells, changes in sleep patterns, physical exertion, weather changes, medications, some foods and food additives.

One outcome from the lack of understanding of causation in migraine is difficulty in treating the disease. The first option regarding migraine is prevention. That is, reducing the frequency and/or severity of migraine episodes. Success has been achieved in this regard connected with a number of the triggers mentioned previously. Reduction in stress, smoking, exposure to strong light, etc., are all actions taken by migraine patients to reduce and/or limit the severity of migraines. The efficacy of addressing the triggers of migraine has a very high level of variability among patients. Perhaps more importantly, the length of time such trigger avoidance is effective also has a high level of variability from patient to patient. That is, elimination of some triggers will have zero efficacy for some patients and long-term efficacy for others, with the majority falling somewhere in between.

Drugs for migraine headaches can relieve the pain and other symptoms of migraine and/or may help prevent future migraine episodes. Abortive treatments are those that seek to reduce or eliminate a migraine once it starts or once the patient feels that a migraine is approaching. Abortive medications are particularly useful in persons with prevalent nausea/vomiting symptoms. Preventive treatments seek to lessen the frequency and severity of migraine attacks and are typically taken on a set schedule, e.g., daily or weekly. Prevention is considered if migraines occur frequently, i.e., more than once per week, or if migraine symptoms are severe. Abortive treatments include triptans and ditans, which specifically target serotonin. Such drugs include almotriptan, eletriptan, frovatriptan, naratriptan, rizatriptan, sumatriptan and zolmitriptan.

Over the counter pain medications and combination pain medications have been used for migraine essentially since their introduction. The most used drugs include those containing ibuprofen, aspirin, acetaminophen, caffeine, isometheptene and dichloralphenazone. Drugs containing mixtures of these compounds are popular, including Excedrin® Migraine which contains aspirin, acetaminophen and caffeine. Ergot alkaloids, including dihydroergotamine and ergotamine, are used to treat migraine often in combination with caffeine and other compounds.

Antagonists to calcitonin gene related peptides (CGRP), discussed further hereinbelow, are also used as abortive treatments for migraine. Abortive treatments for migraine related nausea include chlorpromazine (Thorazine®), droperidol, metoclopramide and prochlorperazine. Drugs for headache pain but not specific to migraine include analgesics, narcotics and barbiturates though these drugs are less ideal due to potential to be habit forming.

Some abortive treatments, especially when used by chronic migraine patients, can lead to worsening of chronic migraine. Overuse of such treatments often results in a secondary headache called a medication overuse headache. Further, such treatments often have well known cardiovascular and gastrointestinal side-effects, e.g., chronic use of nonsteroidal antiinflammatory drugs (NSAIDs) increases risk of peptic ulcer, renal failure, stroke and myocardial infarction.

Preventative treatment medications are more likely to be administered as the frequency or severity of migraine symptoms increase. Some high blood pressure medications have been prescribed as preventative treatments, including beta-blockers such as propanolol, timolol and metoprolol as well as calcium channel blockers such as verapamil. Antidepressant medications such as amitriptylin and nortriptyoline have been utilized. Antiseizure medications like gabapentin, topiramate and valproic acid have been prescribed. Again, calcitonin gene related peptides are discussed further hereinbelow and have also been prescribed as preventative treatment therapeutics for migraine. Injectable botulinum toxin (Botox®) prevents the release of the neurotransmitter acetylcholine from axons near the neuromuscular junction, causing a type of paralysis and is an FDA approved treatment for chronic migraine headache.

Several medical devices are also available for treatment of migraine headache. Some of these devices are based on the premise that migraine causation or symptoms of migraine are related to neuronal activity in the brain and that modulation of this neuronal activity will have an effect on migraine and/or its symptoms. Cefaly® is a small headband device that sends electrical pulses through the forehead to stimulate a nerve linked with migraines. Cefaly® is an electronic transcutaneous nerve stimulation (“e-TNS”) device available over the counter and is approved by the FDA for migraine treatment and prevention. Single Pulse transcranial magnetic stimulators (“sTMS”) are based on the theory that aura in migraine results from a wave of unusual electrical activity called cortical spreading depression. A device that emits relatively strong pulse(s) of magnetic energy may disrupt this wave and thus prevent the onset of aura. One sTMS device is the eNeura sTMS Mini® which is a small device held to the back of the head by the user which emits a short magnetic pulse. Spring TMS® is similar to eNeura sTMS Mini®. Both of these TMS devices are FDA approved. A noninvasive vagus nerve stimulator (nVS) is a hand-held portable device placed over the vagus nerve in the neck that releases a mild electrical stimulation to relieve pain. It appears that several nVS devices have been approved by the FDA for use in treating migraine.

Many migraine patients are encouraged to keep a migraine journal that may assist the patient and their healthcare provider with the diagnosis and the identification of triggers. A highly detailed and frequently updated journal may be a useful tool but the ability of a patient to keep such a journal, even for a short span of time, is highly variable. Things tracked in such a journal include date and time of when the migraine/prodrome started, whether symptoms preceded the head pain, time periods of the four stages, levels of pain, unilateral/bilateral spread, other symptoms accompanying headache, etc. Patterns can be a very helpful tool, including anticipation of what will happen in the future. Diary entries as to how many hours of sleep per night, stress level, weather, food/water/alcohol intake, medications taken, etc., are all helpful things in such a diary, permitting insight into triggers and other migraine factors. Similarly, medications or other treatments attempted for a given migraine and their efficacy is very useful data to track. A number of smartphone apps have sought to take the place of things like a migraine journal with mixed success.

### CGRP Drugs in Migraine

The calcitonin superfamily of peptides includes at least five known members: calcitonin, amylin, adrenomedullin, and two calcitonin gene-related peptides (“CGRP”), CGRP1 (also known as ctCGRP, or CGRP) and CGRP2 (also known as βCGRP).

CGRP and physiological changes linked thereto have been shown to be present in migraine. CGRP is a 37 amino acid vasoactive neuropeptide expressed in both the central and peripheral nervous systems and has been shown to be a potent vasodilator in the periphery, where CGRP-containing neurons are closely associated with blood vessels. CGRP-mediated vasodilatation is associated with neurogenic inflammation, as part of a cascade of events that results in extravasation, i.e., leakage, of plasma and vasodilation of the microvasculature.

Amylin (“Amy”) has specific binding sites in the central nervous system (“CNS”) and is thought to regulate gastric emptying and have a role in carbohydrate metabolism. Adrenomedullin is a potent vasodilator and has specific receptors on astrocytes and its messenger RNA is upregulated in CNS tissues that are subject to ischemia.

Calcitonin is involved in the control of bone metabolism and is also active in the CNS. The biological activities of CGRP include the regulation of neuromuscular junctions, of antigen presentation within the immune system, of vascular tone and of sensory neurotransmission. Three calcitonin receptor stimulating peptides (CRSPs) have also been identified in a number of mammalian species; the CRSPs may form a new subfamily in the CGRP family.

Further to CGRP in particular, the peptide chain of 37 amino acids is produced primarily in peripheral and central neurons. Although technically a hormone, many attributes and functions of CGRP1 are similar to those of a neurotransmitter. In the spinal cord, the function and expression of CGRP1 differs relative to its location of synthesis. Besides its vasoactive functions, CGRP1 can function in transmission of nociception, may contribute to regeneration of nervous tissue, may be linked to pain transmission, is thought to play a role in cardiovascular homeostasis, acts as a chronotype in the heart by increasing heart rate, is known to modulate the autonomic nervous system, has moderate effects on calcium homeostasis and plays a role in ingestion.

The receptor for CGRP1 has more than one part. One part of the receptor is a G protein-coupled receptor known as the calcitonin receptor-like receptor (“CRLR”). The other part is also a transmembrane protein, this one is called a receptor activity-modifying protein (“RAMP”). When RAMP1 interacts with CRLR a CGRP receptor results whereas when a RAMP3 interacts with CRLR a dual CGRP and adrenomedullin receptor results. This results from the RAMP family of polypeptides acting as receptor modulators that determine the ligand specificity of receptors for the calcitonin peptide family members. Unless associated with a RAMP, CRLR is not known to bind any endogenous ligand.

CGRP is a potent vasodilator that has been implicated in the pathology of a number of vasomotor symptoms, such as all forms of vascular headache, including migraines (with or without aura) and cluster headache. Migraine pathophysiology involves the activation of the trigeminal ganglia, where CGRP is localized, and CGRP levels significantly increase during a migraine attack. This in turn, promotes cranial blood vessel dilation and neurogenic inflammation and sensitization. Further, the serum levels of CGRP in the external jugular vein are elevated in patients during migraine headache. Intravenous administration of human ci-CGRP induced headache and migraine in patients suffering from migraine without aura, supporting the view that CGRP has a causative role in migraine.

Possible CGRP involvement in migraine has been the basis for the development and testing of a number of compounds having some impact on CGRP. Triptans are a family of drugs used as abortive migraine medications; about a half-dozen triptans have been approved by the U.S. FDA. The agonist effects of triptans on serotonin receptors in blood vessels and nerve endings result in the inhibition of CGRP. Several proposed compounds, e.g., BIBN4096BS, antagonize the CGRP receptor, thus inhibiting CGRP. A potent small-molecule CGRP antagonist, telcagepant (MK-0974), has been shown to relieve moderate-to-severe migraine attacks, including migraine pain and migraine-associated symptoms.

Erenumab-aooe (AIMOVIG®) is a monoclonal antibody that binds with high affinity to the CGRP receptor, antagonizing the receptor’s function. Erenumab-aooe was first in class of monoclonal antibody therapies for migraine when allowed by the FDA in May 2018. Fremanezumab (AJOVY®) and galcanezumab (EMGALITY®) are both monoclonal antibody based drugs that also antagonizes the CGRP receptor and were approved by the FDA subsequent to erenumab-aooe.

Erenumab-aooe (“erenumab”) is a human immunoglobulin G2 (IgG2) monoclonal antibody that has high affinity binding to CGRP receptor. Erenumab-aooe is produced using recombinant DNA technology in Chinese hamster ovary cells. It is composed of 2 heavy chains, each containing 456 amino acids, and 2 light chains each containing 216 amino acids. Erenumab-aooe is supplied as a sterile, preservative-free, solution for subcutaneous injection. Each 1 mL prefilled single-dose injector, whether autoinjector or glass syringe, contains 70 mg erenumab-aooe, 1.5 mg acetate, 0.10 mg polysorbate 80 and 73 mg sucrose. Recommended dosage is 70 mg once monthly with some patients benefitting from a dosage of 140 mg once monthly.

In a randomized, multi-center, 3-month, placebo-controlled, double-blind study evaluating erenumab as a preventive treatment of chronic migraine, 667 patients with a history of chronic migraine with or without aura were randomized such that 191 received 70 mg erenumab, 190 received 140 mg erenumab and 286 received placebo by subcutaneous injections once monthly for 3 months. Patients were allowed to use acute headache treatments including migraine-specific medications, i.e., triptans, ergotamine derivatives) and NSAIDs during the study. The mean migraine frequency at baseline was approximately 18 migraine days per month and was similar across treatment groups. At both the 70 mg and 140 mg monthly dosages, the change from baseline in migraine days per month was -6.6 days. Further, 39.9% of the 70 mg dosage group and 41.2% of the 140 mg dosage group cut their monthly migraine days by at least one-half.

Blocking intestinal calcitonin gene-related peptide (CGRP) with a CGRP antagonist such as erenumab, a medicine used for migraine prevention, may lead to constipation, which can be severe in some patients. This side effect is the result of the gastrointestinal (digestive) tract containing CGRP proteins. Some studies suggest that CGRP may play an important role in maintaining the movement of the bowels. Most people who develop constipation with erenumab do so after the first injection, but it may also occur later. In the clinical studies involving erenumab, constipation was one of the most common adverse reactions reported, occurring in about 3 out of 100 patients. Higher monthly dosing of erenumab correlates with higher incidence of constipation.

In some people, the constipation with erenumab is severe enough that constipation related complications result. Hospitalization or surgery may be needed in some cases. Thus, making patients aware of the issue, monitoring patients for constipation and dealing with the issue in a timely and effective manner are all important when constipation arises as a side effect.

A treatment protocol may be provided comprising an abortive drug and/or a preventative drug. The abortive drug and/or the preventative drug may be any suitable pharmaceutical compound as described in the present disclosure. The abortive drug may be configured to inhibit at least one of a plurality of a calcitonin superfamily of peptides. In effect, the abortive drug may be adapted to bind with a plurality of calcitonin gene-related peptides (CGRP) receptors. Such binding may reduce migraine symptoms associated with CGRP. However, in alternate embodiments, the abortive drug may be configured to inhibit any family of peptides or any other substance as described herein, wherein such inhibition reduces migraine symptoms. Accordingly, in such an embodiment, the abortive drug may be configured to bind to any suitable corresponding receptor. In an embodiment, the abortive drug may be prescribed to the patient in accordance with a treatment curated by a medical professional. In terms of the treatment protocol, the abortive drug may be prescribed in accordance with an abortive dosage. For example, the abortive dosage may be adjusted to stop migraine symptoms during a migraine attack. In one embodiment, the plurality of the calcitonin superfamily of peptides comprises one or more CGRPs, the one or more CGRPs comprise a 37 amino acid vasoactive neuropeptide, and/or the abortive drug is bound to the plurality of CGRP receptors to mitigate vasodilation.

The treatment protocol may include or may be utilized in conjunction with a client device 120 comprising at least one processor, at least one memory comprising a drug database and computer-executable instructions. The instructions may cause the client device 120 to receive, via the at least one memory, an initial abortive drug regimen. The abortive drug regimen may be prescribed by a health care professional. In one embodiment, the abortive drug regimen may be uploaded to the system from the medical professional, for example, via a portal or other means. However, in another embodiment, the abortive drug regimen may be imported to the system by the patient. The abortive drug regimen may comprise one or more abortive drugs and one or more abortive dosages. For example, the abortive dosage may be a dosage configured to stop migraine symptoms. In further aspects, the abortive drug regimen may comprise any number of variables or characteristics, such as a list of tertiary medications to avoid due to reactions with the prescribed drug, a list of side effects of the drug, and safe storage information.

Further, the drug treatment protocol may be configured to receive user input via an interactive session. The interactive session may be configured for bidirectional communication between the patient and the client device 120. The treatment protocol may include any embodiment of the interactive sessions as described herein. During interactions with the interactive session, the client device may extract medication-relevant information from the user input. In such an embodiment, the medication-relevant information is correlated to drug-specific information stored within the drug database. For example, the medication-relevant information may be components of the user input that are relevant to drug side effects, either negative or positive. As a non-limiting example, the user input may include information pertaining to the user’s day, wherein the user recounts their trip to the grocery store where the user became dizzy in the frozen foods aisle. Accordingly, the protocol may be configured to extract the fact that the user became dizzy from that set of user input. Further, the client device or a server may comprise a database, such as a drug database, wherein such a database comprises each potentially prescribed drug and the correlated positive and negative effects. Thus, by extracting the medication-relevant information from the user input and cross referencing the medication relevant-information with the drug database, the system may determine the effects felt by the user as a function of the drug administration. Thus, the client device 120 may be instructed to analyze, via the at least one processor, the medication-relevant information in view of the drug-specific information to determine whether the initial drug regimen is being followed and whether the medication-relevant information comprises one or more side effects and one or more positive effects, wherein the one or more side effects and the one or more positive effects are correlated to the one or more drugs via the drug database. Further yet, the protocol may be configured to determine, based on the medication-relevant information and the user input, a migraine severity of the user. The migraine severity may be a measure of the migraine severity and/or the patient’s interpretation of the migraine severity. Accordingly, the migraine severity may be a relativistic measurement; for example, evaluated based on previous conditions of the user. In such an embodiment, the System may be configured to track and/or juxtapose conditions of the user over time, as to create relativistic condition measurements. For example, the System may evaluate symptom severity based on the user’s responses to activities. Thus, the System may evaluate fluctuations in such responses to determine severity of migraine. Alternatively, migraine severity may be measured ‘absolutely.’ For example, a group of subjects may be evaluated for migraine severity and the System may compare the conditions of the one or more subjects to those of the user.

The computer-executable instructions may cause the client device to display an alert configured to inform the user to administer the one or more abortive dosages if the migraine severity is greater than an abortive drug threshold. For example, the alert may be a message to a smart phone, an audio alert emitted from the client device, or a vibrational alert. However, the alert may be any suitable means of making the patient aware. In a further embodiment, the system may be configured to determine whether the alerts are effective. If an alert is considered to be ineffective, the system may generate another alert exhibiting different characteristics. The instructions may further cause the client device to analyze, via the at least one processor, the medication-relevant information in view of the drug-specific information to determine whether the initial abortive drug regimen is being adhered to and whether the medication-relevant information comprises one or more side effects or one or more positive effects. In such an embodiment, the one or more side effects and the one or more positive effects are correlated to the one or more abortive drugs via the drug database.

In addition to an abortive drug, the treatment protocol may comprise a preventative drug. The preventative drug may be configured to inhibit at least one of the plurality of the calcitonin superfamily of peptides, where the preventative drug is adapted to bind with the plurality of CGRP receptors. The treatment protocol may further comprise a preventative dosage, wherein the preventative drug bound to the plurality of CGRP receptors mitigates vasodilation. The preventative drug may be the same pharmaceutical as the abortive drug or may be a different pharmaceutical. In an aspect, however, the abortive dosage is different than the preventative dosage. For example, in one embodiment, the abortive drug and the preventative drug may be the same pharmaceutical compound, but the abortive drug dosage may be greater than that of the preventative drug.

The treatment protocol may receive an initial preventative drug regimen, wherein the preventative drug regimen comprises one or more preventative drugs and one or more preventative dosages. For example, the initial preventative drug regimen may be prescribed by a health professional and uploaded, via a portal or other means, by the health professional. However, the initial preventative drug regimen may also be uploaded by the patient. The treatment protocol may display, via the client device, a reminder configured to inform the user to administer the one or more preventative dosages according to the initial preventative drug regimen. Such a reminder may be a push notification on a smart phone, a text message, or another suitable alert.

The treatment protocol may be configured to analyze the medication-relevant information in view of the drug-specific information to determine whether the initial preventative drug regimen is being adhered to and whether the medication-relevant information comprises one or more side effects or one or more positive effects, wherein the one or more side effects and the one or more positive effects are correlated to the one or more preventative drugs via the drug database.

In a further aspect, the computer-executable instructions which, when executed by the at least one processor, cause the client device to generate, via the at least one processor, an activity within the interactive session, the activity configured to increase the one or more positive effects and decrease the one or more side effects, wherein the activity is one of a plurality of activity types, and wherein the activity comprises an activity content; receive, via the at least one memory, an activity response; and analyze, via the at least one processor, the activity response to determine whether the one or more side effects and the one or more positive effects have fluctuated relative to the previously assessed one or more side effects and one or more positive effects. In yet a further aspect, the computer-executable instructions may cause the client device to determine, via the at least one processor, an activity content efficacy and an activity type efficacy; and generate, via the at least one processor, a succeeding activity, wherein the succeeding activity is one of the plurality of activity types according to the activity type efficacy, and wherein the succeeding activity comprises the activity content according to the activity content efficacy. Similarly, the activity content efficacy and the activity type efficacy may be utilized to determine the overall drug efficacy and/or migraine severity. For example, the treatment protocol may utilize the efficacy measurements of activities that were administered during, before, or after, a preventative or abortive drug administration.

In an embodiment, the protocol may further comprise one or more sensors in electrical communication with the client device, the one or more sensors configured to capture the user input and the activity response and transmit said user input and said activity response to the at least one memory.

In a further aspect, the protocol further comprises a server hosting a patient network, the server comprising at least one server processor, at least one server memory comprising computer-executable server instructions which, when executed by the at least one server processor, cause the server to generate, via the at least one server processor, a profile for each of a plurality of users, each profile comprising a digital happiness wallet (“wallet”); receive, from the client device, an activity performance data; populate, via the at least one server processor, the profile and the wallet with the activity performance data; and distribute, via the patient network, at least the profile. The computer-executable server instructions, when executed by the at least one server processor, may further cause the server to select, via the at least one server processor, from the patient network, one or more suggested profiles based on the user information; and transmit, to the client device, the one or more suggested profiles.

Additionally, the computer-executable instructions which, when executed by the at least one processor, may further cause the client device to generate an updated abortive drug regimen based on the initial abortive drug regimen, the one or more side effects, and/or the one or more positive effects. Similarly, the instructions may cause the client device to generate an updated preventative drug regimen based on the initial preventative drug regimen, the one or more side effects, and the one or more positive effects. In one embodiment, the updated abortive drug regimen and/or the updated preventative drug regimen may replace the initial abortive drug regimen and/or the initial preventative drug regimen, respectively. However, in another embodiment, the updated drug regimen(s) are transmitted to a server and/or the original prescribing medical professional. Accordingly, the medical professional may authorize changes to the drug regimen(s) based on the updated regimen(s) as compiled by the treatment protocol client device. In one embodiment, the server comprises a means of enabling the medical professional to simply modify the regimen(s) as they will appear to the patient via the client device 120.

### Computing System for Implementing a Digital Therapeutic

The present invention includes and requires an interactive computing system that provides an environment in which a human user interacts with the computer for the purpose of achieving one or more clinical benefits to the user. The clinical benefit to the user can be relatively direct in nature, such as decreasing the level of depression or decreasing the level of anxiety of the user. Clinical benefits of a somewhat indirect nature may also be achieved. For example, if depression and/or anxiety are significant comorbidities that may amplify the impact of symptoms of another disease, then managing depression and/or anxiety will result in treating the other disease symptoms.

A digital therapeutic regimen may also have the benefit, which may be classified as direct or indirect, of increasing medication compliance and adherence. Medication compliance is defined as how well a patient follows the directions written on a prescription. Medication adherence is related to compliance but involves the level of motivation a patient has in sticking to a therapeutic regimen. Adherence is often impacted by social and environmental influences. Difficult side effects of a drug will have a tendency to negatively impact adherence and compliance. This is because the prevalence and severity of side effects varies among patients and, just as important, patients possess varying levels of motivation regarding sticking to their therapeutic regimen. Whereas minor side effects require minimal motivation to adhere to a regimen, major side effects will require greatly increased motivational basis. Severe side effects may also have an impact on a patient’s ability to properly weigh the costs versus the benefits of a given therapeutic regimen. As an extreme example, the severe side effects of some chemotherapy regimens result in poor adherence and compliance in spite of the huge benefits said regimen has upon the patient.

A more prosaic example of the impact of side effects on adherence is a treatment for migraine that results in constipation. Although it would be hoped that very few patients would tend toward non-compliance with moderate constipation, i.e., choose more migraines to avoid the constipation side effect, human psychology simply does not work this way. Whatever the mental process of a given patient, e.g., choosing the devil you know versus the devil you do not know, there are many examples of drugs having poor adherence and compliance in spite of the relative benefit of the treatment being substantial and the side effect, i.e., cost, being low or moderate. There are a number of potential explanations for this counterintuitive result but, ultimately, much will depend on the individual psychology of a particular patient. Some patients may have an increase in depression and/or anxiety based on a side effect and this increase may, in some portion of patients suffering this side effect, lower the patient’s adherence by having an outsized impact on their cost/benefit calculation.

Other psychological factors may have a significant impact on a patient’s compliance or, perhaps, be utilized in increasing their adherence. For example, mindfulness may be used to encourage the patient to fully appreciate the costs versus benefits of adhering to a medication regimen. By having a positive impact on the patient’s psychology, including increasing their mindfulness, the patient is better prepared for dealing with side effects.

In general, and as described in greater detail herein, the computing system is configured to provide and engage the user in a set of activities and tasks particularly designed and selected for that user to increase the user’s level of happiness and lower their level of anxiety. The system may also be configured to address symptoms of migraine as well as side-effects associated with various migraine treatment regimens.

In accordance with the present invention, the computing system dynamically responds to the user’s actions and feedback, which result from the user’s partial or full performance of certain activities and tasks, and such dynamic responding by the computing system entails interaction that includes demonstration of simulated human emotion and/or human cognitive skill, such as empathy. As will be further described, interaction that includes demonstration of simulated human emotion and/or human cognitive skill results in a more personal and in-context environment with the user, mimicking a human-to-human conversation that, in turn, resulting in a manner of guiding the user that leads to achieving the desired goal.

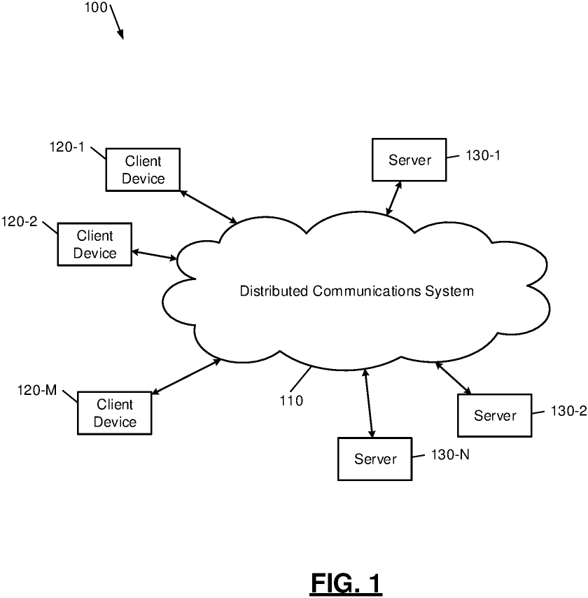

FIG. 1 shows a simplified example of a distributed app 200. The distributed app 200 includes a distributed communications system 110, one or more client devices 120-1, 120-2, etc., collectively, client devices 120 and one or more servers 130-1, 130-2, etc., collectively, servers 130. The distributed communications system 110 may include a local area network (LAN), a wide area network (WAN) such as the Internet, or other type of network. The client devices 120 and the servers 130 may be located at different geographical locations and communicate with each other via the distributed communications system 110. The client devices 120 and the servers 130 connect to the distributed communications system 110 using wireless and/or wired connections. The client devices 120 may include smartphones, personal digital assistants (PDAs), tablets, laptop computers, personal computers (PCs), etc. The servers 130 may provide multiple services to the client devices 120. For example, the servers 130 may execute software applications developed by one or more vendors. The servers 130 may host multiple databases that are relied on by the software applications in providing services to users of the client devices 120. For example, one or more of the servers 130 execute an application that implements the online service including the dialogue management system of the present disclosure.

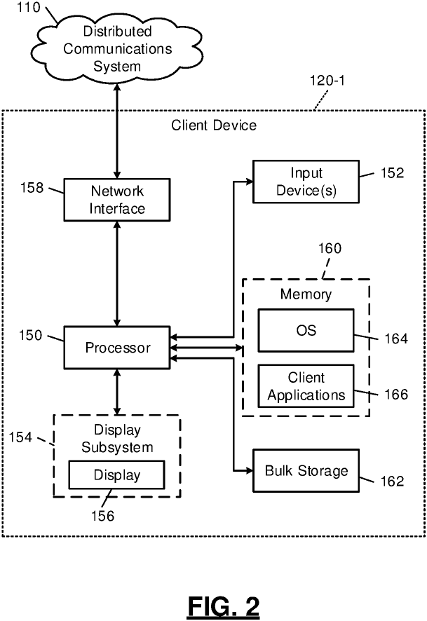

FIG. 2 shows a simplified example of the client device 120-1. The client device 120-1 may typically include a central processing unit (CPU) or processor 150, one or more input devices 152, e.g., a keypad, touchpad, mouse, touchscreen, etc.), a display subsystem 154 including a display 156, a network interface 158, memory 160, and bulk storage 162. The network interface 158 connects the client device 120-1 to the distributed app 200 via the distributed communications system 110. For example, the network interface 158 may include a wired interface (for example, an Ethernet interface) and/or a wireless interface (for example, a Wi-Fi, Bluetooth, near field communication (NFC), or other wireless interface). The memory 160 may include volatile or nonvolatile memory, cache, or other type of memory. The bulk storage 162 may include flash memory, a magnetic hard disk drive (HDD), and other bulk storage devices. The processor 150 of the client device 120-1 executes an operating system (OS) 164 and one or more client applications 166. The client applications 166 include an application that accesses the servers 130 via the distributed communications system 110. The client applications 166 include an application that accesses the online service including the dialogue management system executed by one or more of the servers 130.

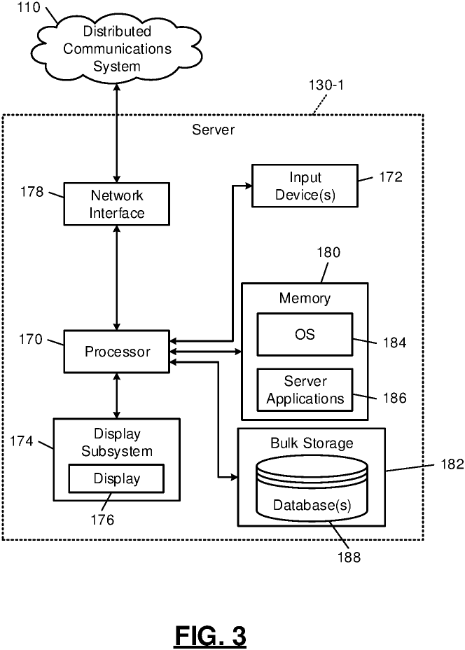

FIG. 3 shows a simplified example of the server 130-1. The server 130-1 typically includes one or more CPUs or processors 170, a network interface 178, memory 180, and bulk storage 182. In some implementations, the server 130-1 may be a general-purpose server and include one or more input devices 172, e.g., a keypad, touchpad, mouse, and so on) and a display subsystem 174 including a display 176. The network interface 178 connects the server 130-1 to the distributed communications system 110. For example, the network interface 178 may include a wired interface, e.g., an Ethernet interface) and/or a wireless interface, e.g., a Wi-Fi, Bluetooth, near field communication (NFC), or other wireless interface). The memory 180 may include volatile or nonvolatile memory, cache, or other type of memory. The bulk storage 182 may include flash memory, one or more magnetic hard disk drives (HDDs), or other bulk storage devices. The processor 170 of the server 130-1 executes an operating system (OS) 184 and one or more server applications 186, which may be housed in a virtual machine hypervisor or containerized architecture, which include the online service and the dialogue management system of the present disclosure. The term “application” or “app” is used extensively herein and includes the online service and similar related concepts. The bulk storage 182 may store one or more databases 188 that store data structures used by the server applications 186 to perform respective functions.

The online service or app is a science-based online service and social community for engaging, learning and training the skills of happiness and related skills for improving mental health attributes. The app can be offered through a variety of computing devices including smartphones, tablets, laptops, etc. The app is based on a framework developed by psychologists and researchers in areas such as positive psychology and neuroscience. The app assists users in the development of skills such as, for example, Savor, Thank, Aspire, Give, and Empathize (or S.T.A.G.E.^{™}). The app includes an additional happiness skill called Revive that is concerned with physical wellness. Throughout the present disclosure, references are made to the STAGE skills for convenience only, and such references should be understood to include the sixth Revise skill. Each skill may be developed using various activities, ordered in increasing skill level, that gradually unlock as the user progresses in building that skill. Users of the app may be given a range of activities from the STAGE skills, from reflective blogging and science-based games and quizzes, to real-life tasks that the users are asked to perform and report back on. Each activity is backed by scientific studies that may be directly accessible by the user via links provided by the app in the recommended activities.

The activities may be offered to users in several ways. One such offering described below focuses on “tracks” that include sets of activities programmed to address a specific life situation or goal, e.g., “Cope better with stress;” “Enjoy parenting more”, etc. Beginning the app, users may complete self-assessments that give them their initial happiness level as well as an initial recommended track. Alternatively, as described in detail below, a particular order of tracks designed to address a particular need set of a user may be implemented. The term ‘need set’ may involve a condition, e.g., migraine, suffered by the user and the symptoms, side effects and comorbidities associated therewith. Tracks may be organized into modules of several tracks with modules also being determined based on need set. When users finish a track part, users may win, for example, a badge that represents their level of activity in that track part.

As users perform their activities, users may create activity posts that are saved in their personal profile and build up a ‘digital happiness wallet’ they can reflect on. Posts may include the type of activity performed by the user, any text and images the user added, other people involved, if any, as well as the time and location for the post. When the activity is a conversation performed with the dialogue management system, a post may include a summary of record of the conversation. Posts also may appear on various activity feeds on the service, which allows other users to read, draw inspiration from, and offer encouragement in the form of comments and likes. Users may also follow activities posted by other users they find interesting if those users allow themselves to be followed or mark their post ‘public.’ Periodically, the app may make suggestions for users to follow other users whose profiles match in terms of demographics and psychographics, as well as level of activity on the site and other criteria.

Periodic, scientifically-designed assessments are an important part of the app and may track a number of relevant parameters related to conditions, symptoms, side-effects and comorbidities suffered by the user. These parameters may be compared to past levels. Over time, the online service may build graphs for the user, comprising of activities, people, places, and things correlated with the impact they had on the parameters being tracked for user. This information may be used to optimize the user experience and the activities the app suggests.

Benefits provided by the app include: clarity, e.g., 5 skills, level progression), integrated self-assessments, e.g., provides self-insights, recommends tracks & activities), progress measurement, e.g., periodic happiness measurements allow the users to monitor their progress), guided experience, e.g., four week track experience optimizes habit formation, enables continued focus on a specific topic, e.g., parenting, stress)), flexible, e.g., track structure allows the users to pick the activities and tasks they prefer from a wider selection of options), personalized, e.g., activity recommendations are based on past user behavior and preference), integrated social experience, e.g., users share and follow, like and comment on other users’ posts), increasingly challenging, e.g., as the users progress, tracks require increased number of activities and higher level of challenge), entertaining, e.g., variety of activity types, track content), extendible in several dimensions, e.g., content: new tracks and track content (tasks, quizzes, polls etc.), activity types: adding new games and activity types, framework: adding new skills), and multi-screen, e.g., web, mobile accessibility).

The app employs a science-to-action framework, provides sustained guidance, allows users to grow visual environments by interacting with them directly, provides contextual social interaction, e.g., users socialize around contextual activity posts prescribed to others, provides activity variety, e.g., real-life, reflective and gaming activities, provides measure-act-measure loop, e.g., allowing users to track their progress as they go, and provides an efficient and versatile dialogue management system that uses a 3-tier architecture to facilitate dialogues about multiple activities performed by multiple users using the least amount of data structures.

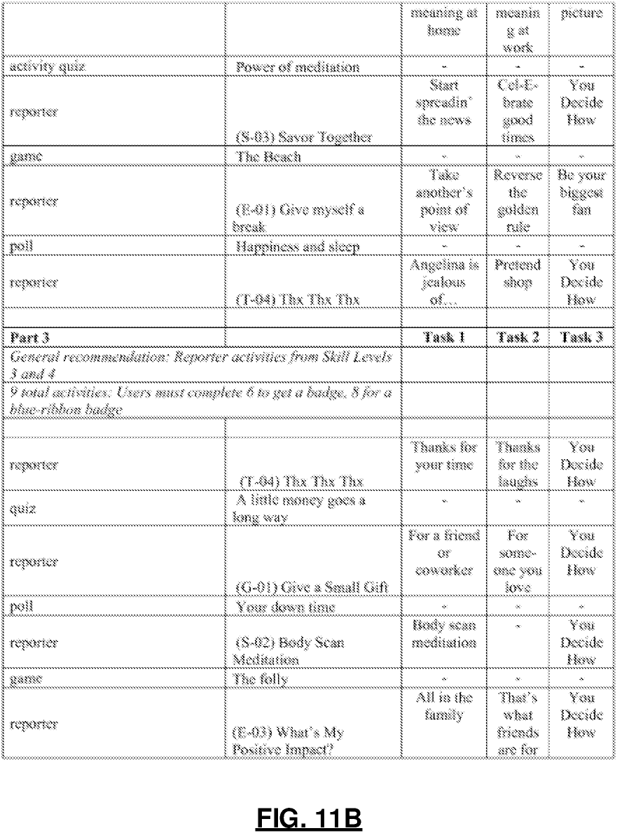

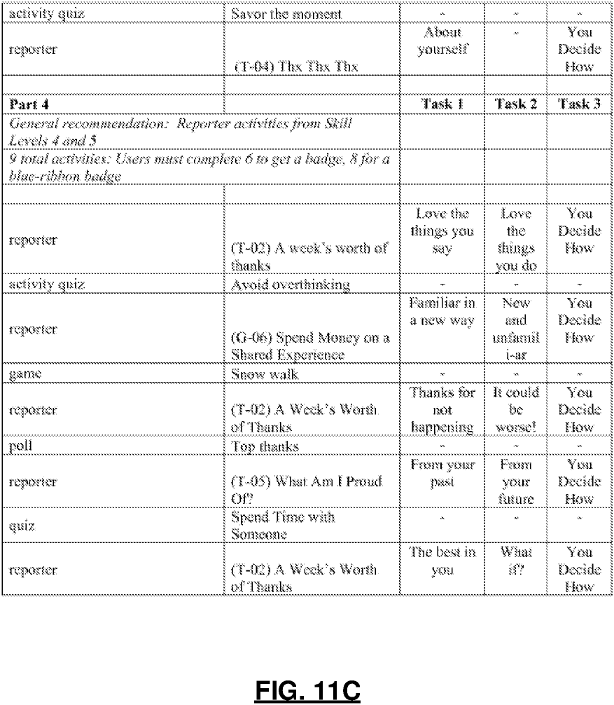

The tracks, modules, activities, and tasks offered by the app are now described in further detail to enhance understanding of the dialogue management system. Tracks are sets of activities that are programmed together to address specific life situations, goals, or concerns that users have. Each track is composed of multiple parts (described below; also see FIGS. 11A-11C). The number of activities and their level of difficulty may be set to increase as the user progresses.

The following are examples of rules that may be used to govern the tracks. Users may be afforded a set time period during which to complete a track part and thus earn badges. Badges may be regular or honors badge, depending on the number of activities they completed. Users may be allowed to extend beyond the set time period and still win the regular badge. If a user reaches the regular badge threshold the user is allowed to ‘win’ the regular badge and move to the next part, or continue for the honors badge. This allows users to skip the remaining activities and win the regular badge if they prefer.

At any time, multiple activities may be available for the user to perform with one or more being ‘queue-locked,’ which means that if the user performs an available activity, it will make the ‘queue-locked’ activity become available. Each day, for example, three time-locked activities become ‘queue-locked,’ and queue-locked activities become available up to a limit of four available activities. This limit of four available activities is intended to avoid showing the users too many available activities when they next log in.

Every activity a user completes creates a post that may be added to the user’s profile. Users can mark their posts private, i.e., only visible to them and not visible to others) or viewable to other people (people who follow them and people doing the track in group mode with them). As part of social interaction, users can view the shared posts of other people who are following the track and can like or comment on them or follow the authors of those posts. Users can like and comment on posts to encourage each other and discuss their contents.

‘Career and money’ tracks include activities directed to the following aspects: appreciate what i have (currently available), reduce on-the-job stress, get energized about my job, stay upbeat while out of work, balance work and home life, and control my spending habits.

‘Family and kids’ tracks include activities directed to the following aspects: enjoy parenting more, better cope with new parenthood, better adjust to becoming an empty nester, forgive and forget feud (with a family member), and better cope with the stresses related to my aging parents.

‘Leisure and friends’ tracks include activities directed to the following aspects: be more socially connected, talkers and listeners, explore the art in happiness, find more “me” time, and be a better friend.

‘Love and intimacy’ tracks include activities directed to the following aspects: feel more loved by my partner, feel and be more devoted to my spouse, fight less and love more in my relationship, get over a broken heart, and feel hopeful to start dating after divorce.

‘Mind and body’ tracks include activities directed to the following aspects: cope better with stress, nurture my body and soul, come to terms with getting older, feel healthier, be more optimistic about my potential, and find more purpose and meaning in my life.

Each part of a track may include a balanced mix of ‘reporter’ activities and ‘light’ activities. The reporter activities may gradually increase in difficulty as a user progress through each of the four parts. Light activities may include: games, e.g., mini games, such as hidden object “mindfulness” game, training the user on a specific happiness skill), quizzes, e.g., multiple-choice or true/false questions about a happiness topic), activity quizzes, e.g., users read a science paragraph about an activity and are quizzed with multiple-choice questions at the end), and polls, e.g., polling users’ opinion about a related topic and showing them community’s vote breakdown). Reporter activities fall into two categories: “essay” or “do” activity, which asks users to reflect on a subject and make a log entry, e.g., reflective microblogging: users are asked to reflect on a topic and write down their thoughts, like what they are grateful for, what they look forward to, taking another person’s perspective, etc.); and “plan-do” activity, which asks user to plan and perform an action in the real world, then come back and report on how it went, e.g., write about his/her experience in a savoring exercise)). The conversational activities, i.e., the conversations performed with the dialogue management system) are different than reporter activities.

A mix of about 50% “reporter” activities and 50% “light” activities may be used in each track part to avoid overwhelming the user. The online service allows for an activity to appear more than once in a track if it’s a crucial activity for the track theme and there are new/different suggested tasks for each use. The number of activities per track part is flexible.

For example, a 7-day sequence of every track part includes a narrative purpose and a feel as if it has a beginning, middle, and an end that gives the user a sense of accomplishment. In the first days of a track part, the activities jump-start a key positive emotion the user will need for subsequent activities or asks the user to try something new, intriguing, fun, or funny-which rattles the user out of her funk and gets her in a good mood for what’s next. In the middle of a track part, the activities build on (or complement) previous ones. An activity may be introduced that needs some extra thought or action. By day 4 or 5, the user feels a little more committed or motivated and willing to take on slightly more demanding activities. In the end, on the last day of a track part, users want something that’s fun, easy or inspiring. Accordingly, unfamiliar/demanding tasks are avoided. The users anticipate a feeling of accomplishment but is intrigued enough to commit to the next part of their track.

The goal of the tracks is to create an appealing balance between activities that can be completed immediately by writing after a few minutes of reflection versus activities that require action (and in some cases, pre-planning) before reporting on how it went. In general, easier (levels 1 and 2) activities are programmed towards the beginning of a track (parts 1 and 2), and as a user progresses to the later parts of a track, the activities become more difficult (levels 4 and 5 activities), but this is not required. Users are awarded badges based on how many activities they complete in each part of a track. The online service offers special badges for each part of a track.

Users interacting with the app may start off at level-1 in all skills. As they complete activities they may progress in each skill from level-1 to level-2, etc. New activities, self-assessments, and other options may unlock as the user reaches a higher level. For each skill, the app offers relevant, science based activities that train the user in an entertaining way. As the users level up in a skill, they unlock new activities (level 1 to level 5 activities may be made available in each skill). Each activity provides the user with several alternatives for completing the activity (“Suggested Tasks”) to pick from. Users can view an explanation of “why it works”: a short summary of the science behind that activity, complete with links to the actual study this activity is based on.

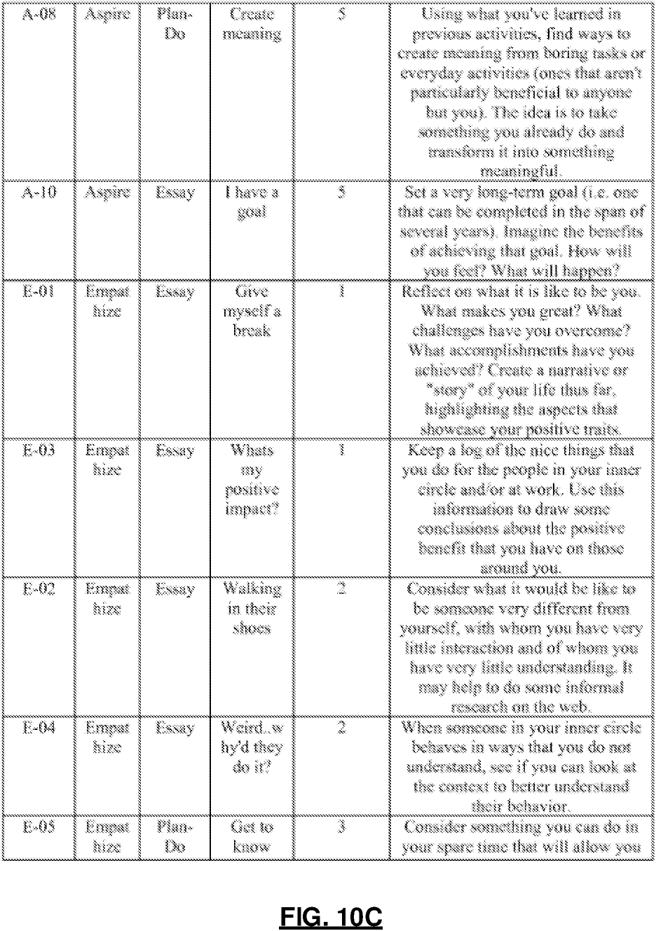

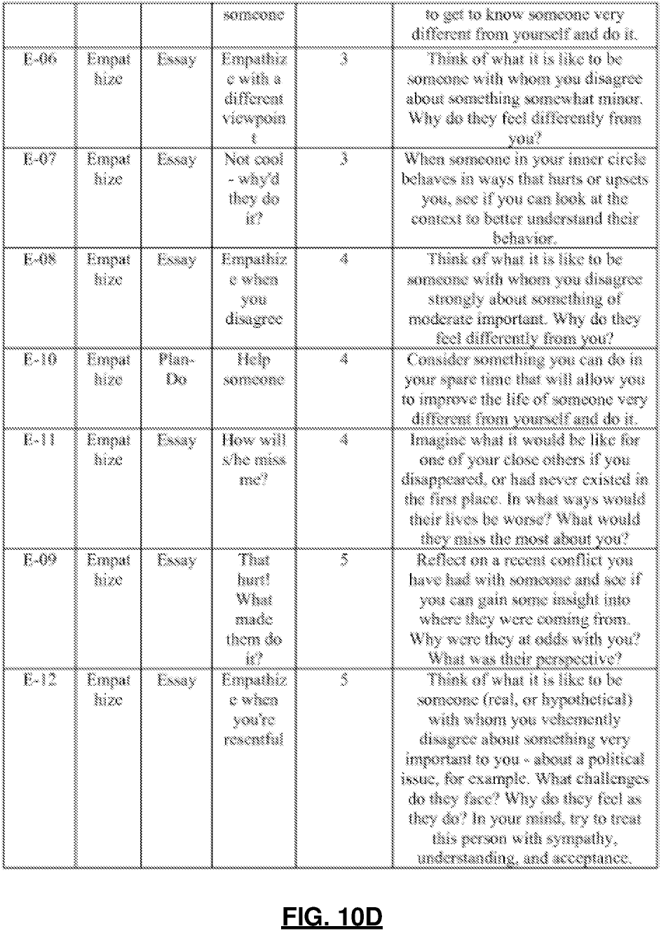

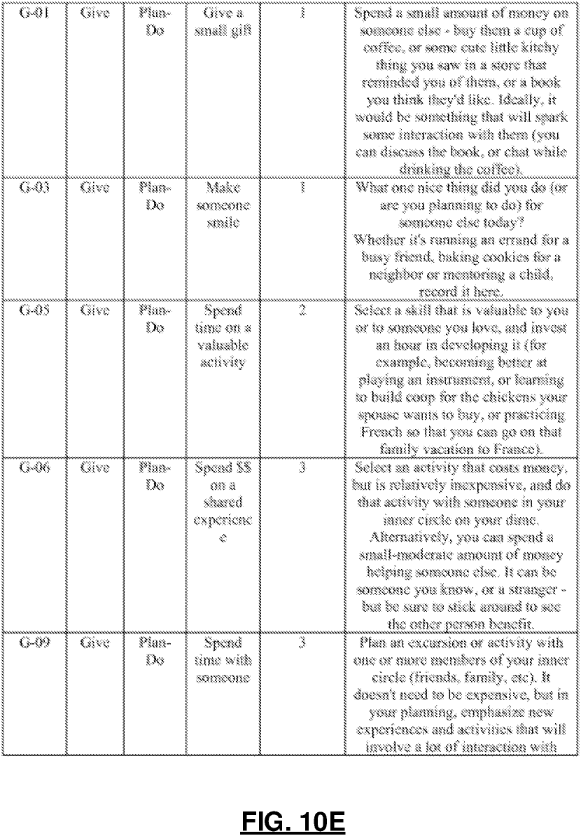

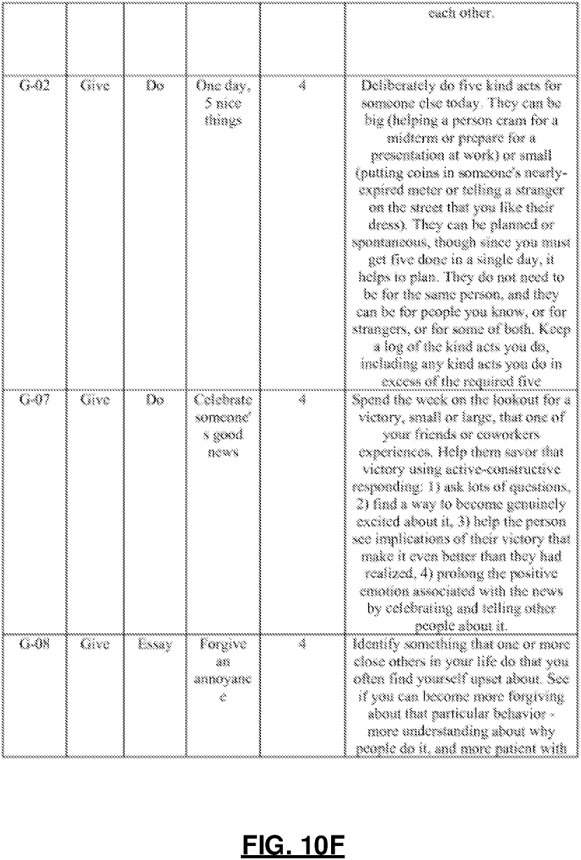

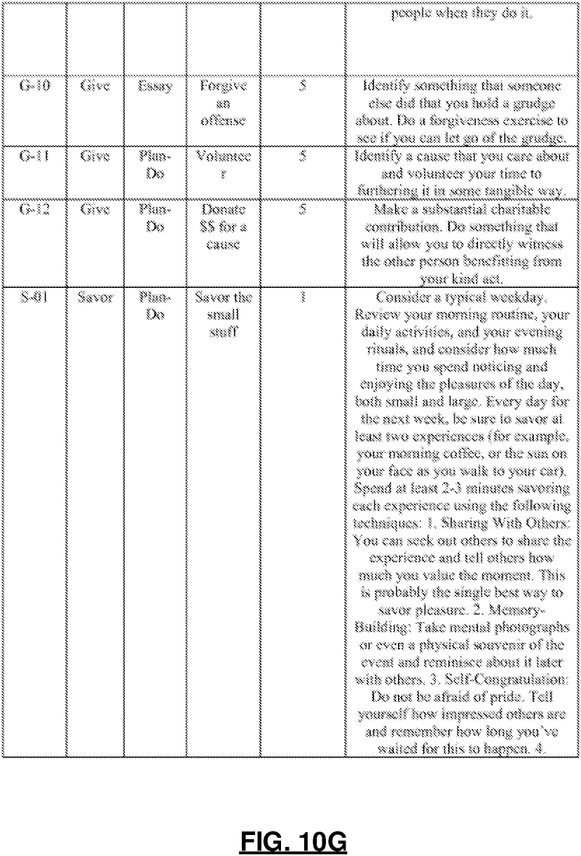

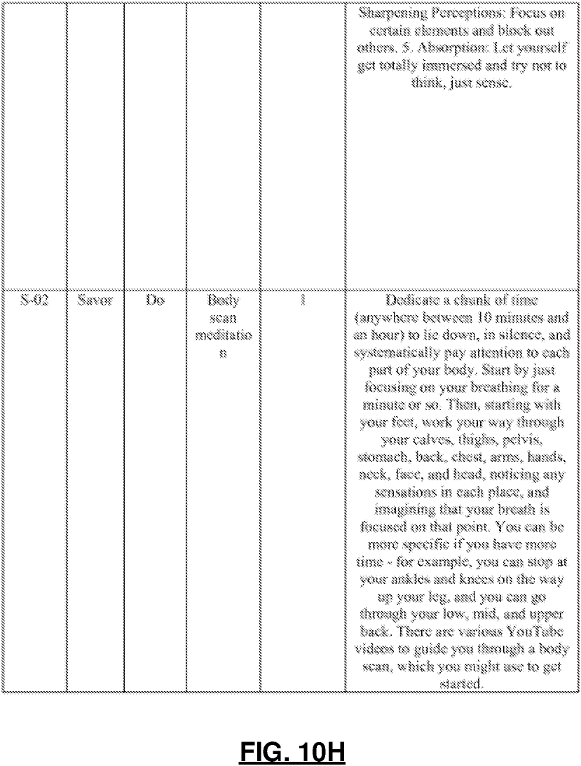

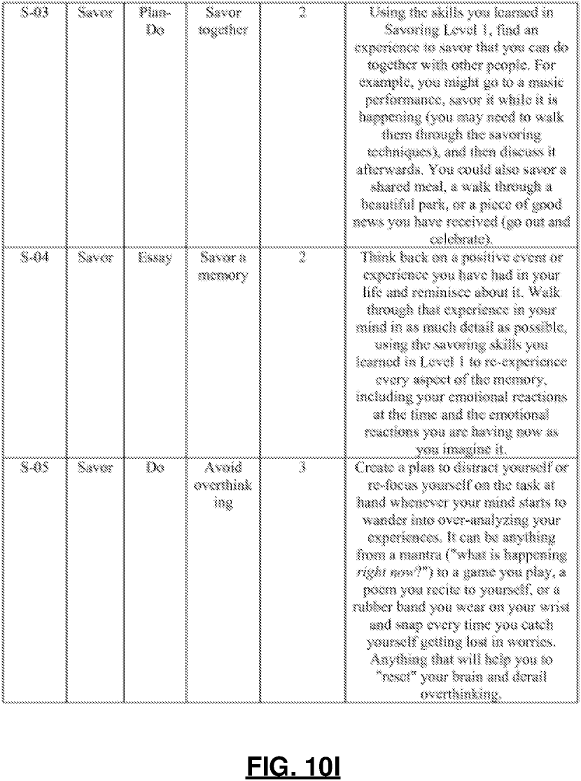

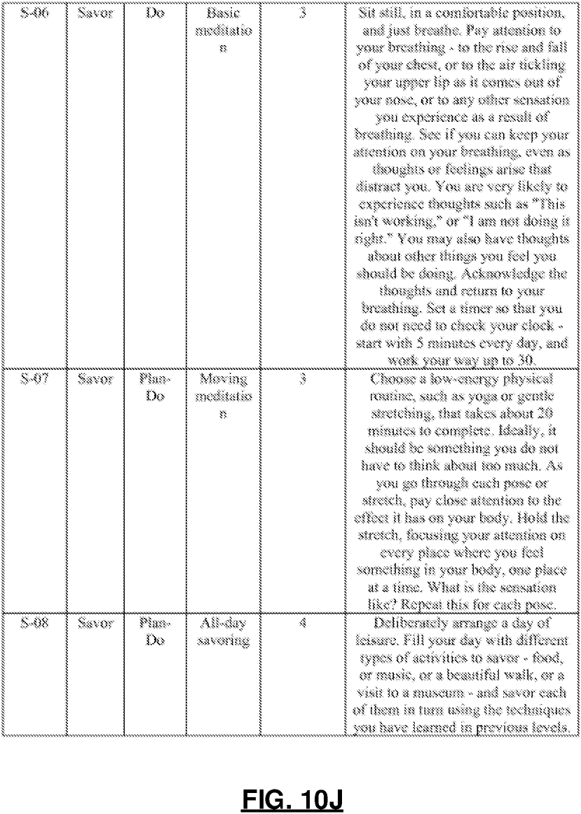

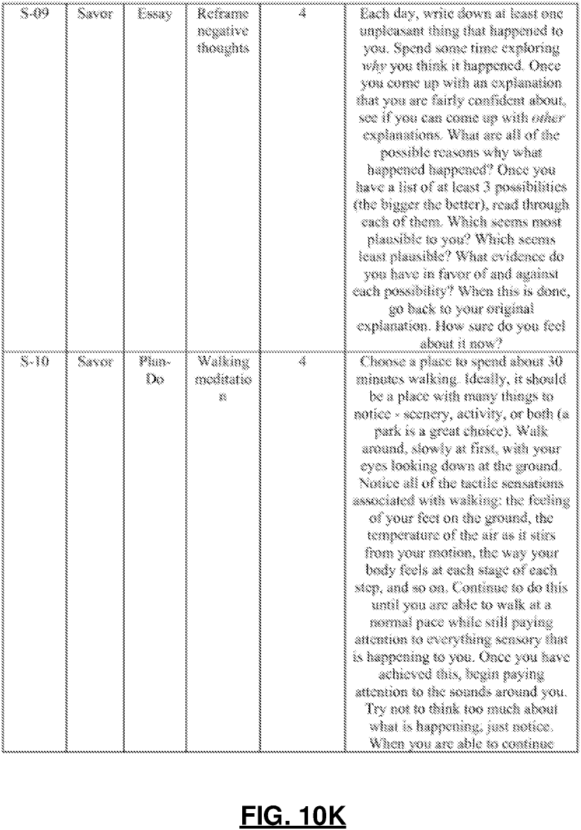

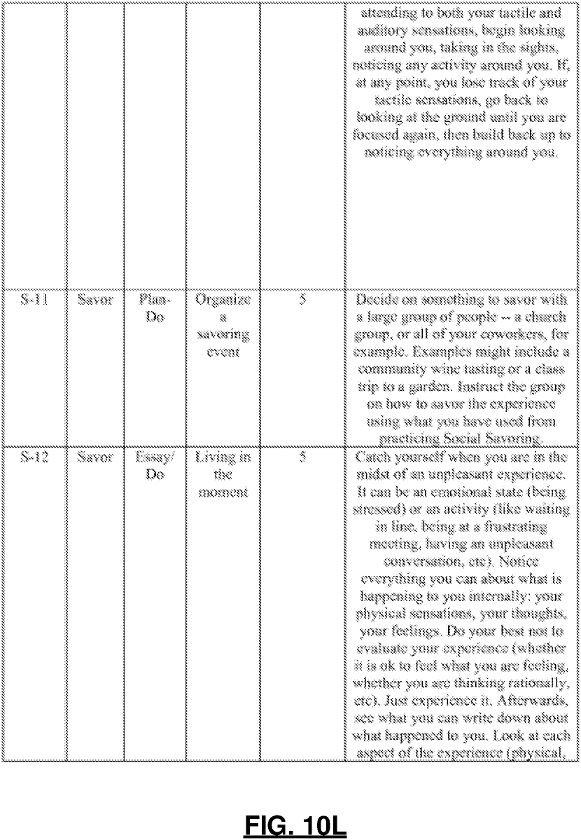

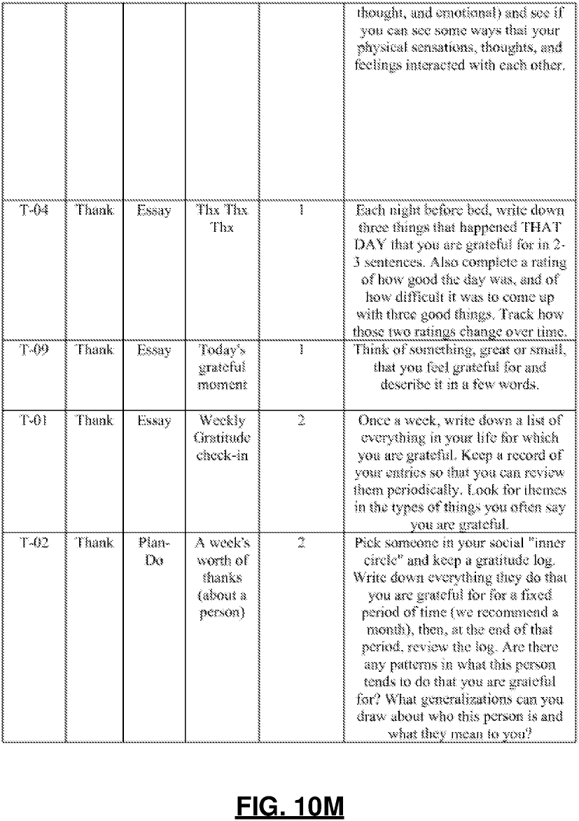

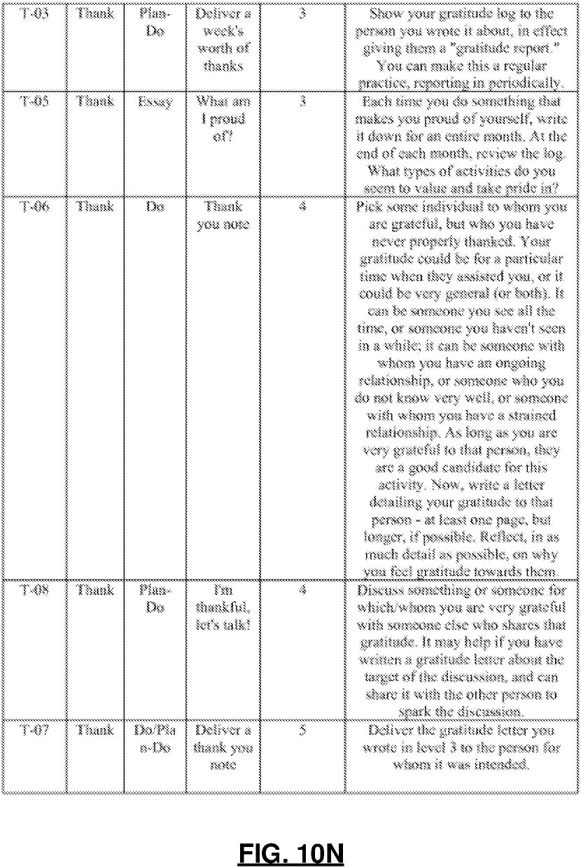

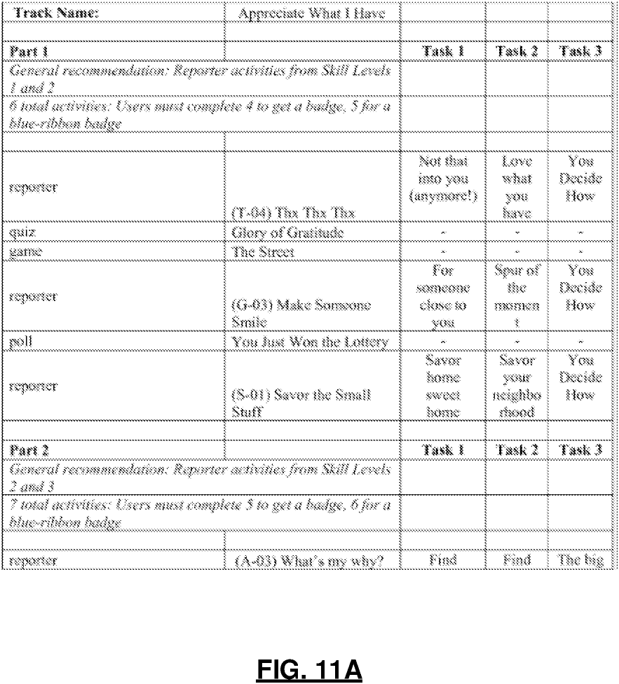

The STAGE framework of the app captures the essence of the science of positive psychology and allows for presentation in an accessible way. The STAGE framework of the app offers different types of science-based activities to users. The app provides nearly sixty science-based activities in various tracks to help users build the following five essential happiness skills: (1) Savor—Noticing the goodness around you and taking time to prolong and intensify your enjoyment of the moment. Savoring can involve the past (reminiscing), the present (mindfulness), or the future (positive anticipation); (2) Thank—Practicing gratitude; identifying and appreciating the things we have and the people in our lives; (3) Aspire—Feeling hopeful, having a sense of purpose and meaning in our lives, being optimistic; (4) Give—Performing acts of kindness; being generous and forgiving; and (5) Empathize—Imagining and understanding the emotions, behaviors, or ideas of others; having compassion. See FIGS. 10A-10N for details. Other and modified details regarding tracks are presented hereinbelow.

The framework of the app may provide multiple suggested tasks for each activity. For example, once the “reporter” activities are determined for each track part, the app provides 2-3 suggested tasks for each activity. These tasks retain the essence and the science of the proven intervention activity, but make sense within the theme of the track. The tasks are fun, and yet give clear and concise directions. A user needs to pick one of these tasks to complete in order to get credit for the activity. That is, users only need to complete one of the task options in order to get credit for a given activity. When a user selects an activity, s/he can choose one of the two suggested tasks or a third “You Decide How” (YDH) option. Each suggested task is accompanied by a “Why It Works” section, which includes science references and explains why the activity is useful and how it relates to happiness. Below are some examples of sample activities and suggested tasks. Comprehensive lists of tracks and activities are provided both in a table shown in FIGS. 10A-10N as well as hereinbelow. An example of a track and its activities and tasks is shown in a table in FIGS. 11A-11C.

For example, for the track Feel More Loved by My Partner, and activity Today’s Grateful Moment [Skill: Thank], a Suggested Task #1 may include the following. Name: The Little Stuff Counts, e.g., think of the reason you first fell in love with your partner or spouse—a trait or characteristic he/she still holds today. It could be his sense of humor, her kind generosity, or maybe his sex appeal. Write down some thoughts and spend a minute appreciating those same traits today. A Suggested Task #2 may include the following. Name: Thanks, Partner!, e.g., think of one good thing that happened today involving your partner or spouse. Write it down and add a few details about how it made you feel and the role you played, if any, in the positive experience. A You Decide How (YDH) task may include the following. For example, think of something, great or small, that you feel grateful for and describe it in a few words. Add a photo too if desired.

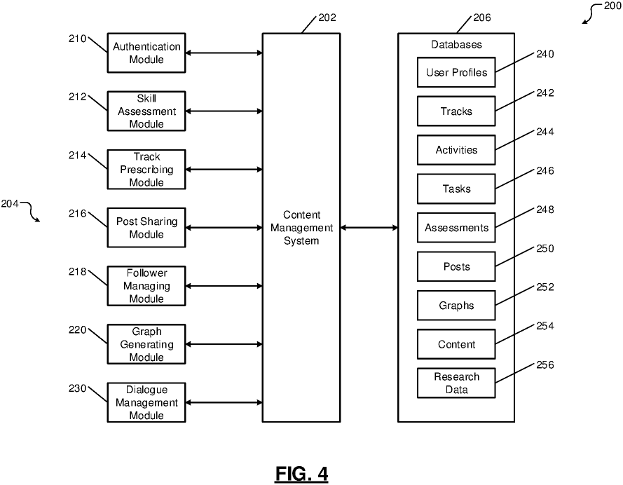

FIG. 4 shows a block diagram of the app described above, which is shown as the app 200. The app 200 comprises a content management system (CMS) 202, a plurality of modules 204 controlling various features and aspects of the app 200 described above, and a plurality of databases 206 associated with and utilized by the respective the plurality of modules 204 and the CMS 202. The CMS 202 manages the overall content provided by the app 200 to the users of the app 200 using the plurality of modules 204 and the plurality of databases 206.

The plurality of modules 204 comprises an authentication module 210, a skill assessment module 212, a track prescribing module 214, a post sharing module 216, a follower managing module 218, a graph generating module 220, and a dialogue management module 230. The authentication module 210 establishes user accounts and controls the users’ access to the app 200. The skill assessment module 212 assesses a user’s skills initially when the user signs up and later periodically as the user performs the prescribed activities. The track prescribing module 214 prescribes the tracks and modifies the tracks to the users according to their skill assessments as described above. The post sharing module 216 manages publication of the posts shared by the users, e.g., keeping them private or publishing them depending on the users’ preferences, handling the likes and comments on the posts by other users, etc.). The follower managing module 218 manages the follower recommendations to the users based on profile matching as described above. The graph generating module 220 generates the happiness graphs as described above. The dialogue management module 230 conducts dialogues between the users and the app 200 and includes the dialogue management system as described below in detail.

The plurality of databases 206 comprises a database for each of user profiles 240, tracks 242, activities 244, tasks 246, assessments 248, posts 250, graphs 252, content 254, and research data 256. The app 200 provides content to the users of the app 200 using the plurality of modules 204 and the plurality of databases 206 under the control of the CMS 202.

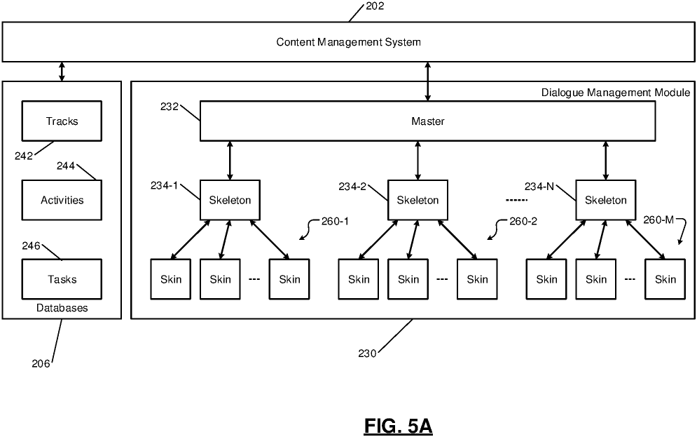

FIGS. 5A and 5B show the dialogue management system 230 in further detail. FIG. 5A shows the dialogue management system 230 having a 3-tier or 3-layer architecture. FIG. 5B shows an example of a dialogue box (or a dialog box) 270 on a user’s device, e.g., a client device 120-1 shown in FIGS. 1 and 2). Throughout the present disclosure, the various “dialogue files” can also be called the respective “dialog files.”

In FIG. 5A, the dialogue management system 230 includes a single master dialogue file (also called a master file or a master) 232, and a plurality of skeleton dialogue files 234-1, 234-2 to 232-N, where N is the number of activities 244. N may be 60, 100 or even higher. These may collectively be called skeleton dialogue files 234, skeleton files 234, or skeletons 234. For each of the skeleton dialogue files 234, the dialogue management system 230 includes a plurality of skin dialogue files 260-1, 260-2 to 260-M. These may collectively be called skin dialogue files 260, skin files 260, or skins 260. The skin dialogue files 260 may include You Decide How (YDH) skin files and task skin files. Throughout the present disclosure, an individual skin file (YDH or task), a YDH skin file, and a task skin file are also referenced by the numeral 260. The dialogue management system 230 and its components, which include the master dialogue file 232, the skeleton dialogue files 234, and the skin dialogue files 260 are described below in further detail.

The dialogue management system 230 allows users to engage in a dialogue with the app 200 about an experience related to performing a prescribed activity 244. Dialogue boxes are generated using a tiered system of files, each with a unique purpose. An example of a dialogue box shown in FIG. 5B. Specifically, the dialogue boxes may be created using three sets of tiered or layered files: a single master dialogue file (master) 232, a plurality of skeleton dialogue files (skeletons) 234, and a plurality of skin dialogue files (skins) 260. The dialogue management system 230 that creates the dialogue boxes may include three layers of files—master, skeleton, and skin (MSS)—and can also be called a MSS system. Note that theoretically there can be multiple master files 232; however, practically, having a single master file 232 simplifies the design of the dialogue management system 230. Alternatively, the master file 232 may be eliminated from some or all dialogues.

While a track 242 may include many activities 244, the dialogue management system 230 includes a hierarchical architecture that leverages some amount of overlap that exists across the activities 244. The dialogue management system 230 may include a single master file 232 for all the activities 244, one skeleton file per activity 244, and one skin file 260 per task 246. The master dialogue file 232 may include the entire and complete markup language or script based structure that to run any dialogue, i.e., for each activity 244 and task 246.

The master dialogue file 232 may, they need not, be a JavaScript Object Notation (JSON) file or an Extensible Markup Language file. The dialogue management system 230 may have a master dialogue file 232 that represents a set of capabilities of the dialogue management system 230. The texts in the prompts, buttons, choices, and responses in the master dialogue file 232 may be fairly generic. For example, in the master dialogue file 232 a response after a user makes a single choice might be “Response to first choice.” This allows the master dialogue file 232 and its HTML based structure to work in any context for any activity 244.

A skeleton dialogue file 234 represents the specific structure for an activity 244, e.g., a skeleton can be designed for S-01 Savor the Small Stuff). The skeleton dialogue file 234 is a JSON file that makes selected references to the HTML structure in the master dialogue file 232 through the use of “include” statements.

A skin file 260, i.e., one of the skin files 260 corresponding to the skeleton file 234 associated with the activity 244) represents actual text to be presented when running a skeleton dialogue file 234 as well as the specific names for variables called life graph variables (LGVs) to be saved for a skeleton dialogue file 234. A skin file 260 is a spreadsheet, a comma separated value (CSV) file or similar data file that specifies the location of strings of text and the text to be used in a dialogue.

The dialogue management system 230 includes two layers of skins 260. Every skeleton dialogue file 234 has an associated overview or You Decide How (YDH) skin file 260. Additionally, a task skin file 260 can also be assigned to a specific task 246, e.g., there would be a specific task skin 260 for S-01-T-27 Smell the Roses).

Running a dialogue may involve identifying a skeleton dialogue file 234 (for example, the skeleton for S-01 Savor the Small Stuff) and a skin file 260 (for example, the skin for S-01-T-27 Smell the Roses). The activity base skin can contain instructions for how to further customize. “Compiling” the dialog uses the master and skeleton assets. Once a dialog is compiled, it is no longer dependent. That is, the master and skeleton and skin could be deleted and the hpml dialog would run just fine. This is true in that the MSS artifacts are used to produce the runtime artifact.

One way to initiate a dialogue involves the master 232, the skeleton 234, and the skin 260 being combined or compiled offline in the CMS 202. A potential optimization would do this in runtime on demand at the time of invocation of the dialogue. The advantage of the former way is that the availability of a full development environment allows the CMS 202 to manage different versions of each master 232, the skeleton 234, and the skin 260 and identify and debug errors if compilation fails.

More specifically, the master dialogue file 232 is sometimes a single file. For example, only one version of the master dialogue file 232 may exist on the server, i.e., in the app 200) at a given time. The master dialogue file 232 can be edited and updated over time, e.g., via the CMS 202), but in ways that overwrite the prior version. The master dialogue file 232 includes all of the core logic needed to determine and lay out the flow of any dialogue that can occur on the dialogue management system 230. The master dialogue file 232, therefore, is comprehensive and non-specific.

For example, the master dialogue file 232 may include the code necessary to run any language modeling and analysis algorithms, performing tasks such as the natural language classifiers (NLCs), Named Entity Recognition, Sentiment Analysis, and Linguistic Style Analysis and Transformation. For example, such algorithms include but are not limited to machine learning, deep learning, neural networks, statistical pattern recognition, semantic analysis, linguistic analysis, and generative models. A final user-facing dialogue may rely on the analysis of user input, e.g., one or two NLCs).

Every potential choice point that can occur in the flow of a dialogue may be coded into the master dialogue file 232. The master dialogue file 232 may include placeholder text that is very broad and generic, e.g., “Response to user”; or e.g., choices for the user can be “Choice 1” and “Choice 2”). Alternatively, the default text, where breadth is not required, can be specific, such as ending the dialogue with “Goodbye” or offering the user choices such as “Yes” and “No”.

Skeletons 234 and skins 260, i.e., the skeleton dialogue files 234 and the skin dialogue files 260, are where specific conversations and interactions with the user are often designed. The dialogue management system 230 may include a skeleton dialogue file 234 for each core activity 244 offered to the users, e.g., the app 200 includes nearly 60 activities. A skeleton dialogue file 234 may be a decisive, singular manifestation of the conversation flow offered by the master dialogue file 232. For example, if the objective is to interview the user about a relationship with a person in the user’s life and the user’s favorite things about that person, the skeleton dialogue file 234 for this interview can clearly delineate the flow for this conversation. The flow in the skeleton dialogue file 234 is deterministic, such that a series of given inputs from the user create a specific, exact conversation with the dialogue management system 230. However, the flow in the skeleton dialogue file 234 is dynamic, and a different set of user inputs can create a different conversation with the dialogue management system 230.

A skeleton dialogue file 234 may utilize only a small portion, e.g., 20% or 10%, of the dialogue portions or sub-dialogues defined in the master dialogue file 232. A skeleton dialogue file 234 may also use the dialogue portions of the master dialogue file 232 more than once. No specific text is determined by the skeleton dialogue file 234. So the skeleton dialogue file 234 can carry over the default text defined by the master dialogue file 232.

Furthermore, there can be an overlap between some of the activities 244. In such instances, the skeleton dialogue files 234 for such overlapping activities 244 can utilize the same or similar dialogue portions of the master dialogue file 232. Further, these dialogue portions in the master dialogue file 232 themselves can be reduced in number based on the overlap in some of the activities 244, which results in optimization in the design of the master dialogue file 232 and which provides additional synergy between the skeleton dialogue files 234 and the master dialogue file 232.

A skin dialogue file 260, i.e., each one of the skin dialogue files 260 includes a list of “specifics” which describes the exact sentences and phrases to be used by the dialogue management system 230 at each point in the conversation flow described by a given skeleton dialogue file 234. Skin dialogue files 260, therefore, are inherently tied to a specific skeleton 234 and are not paired with other skeletons 234. The dialogue management system 230 includes a skin dialogue file 260 for each specific task 246 for an activity 244 offered to users by the app 200. For example, for the nearly 60 core activities, the dialogue management system 230 includes anywhere from dozens to hundreds of skin dialogue files 260 for each activity 244.

In some cases, the default text in the master dialogue file 232 can suffice, such as giving the user a choice between “Yes” and “No”. In these cases, the skin dialogue file 260 can include an indication such as a null entry, allowing the text to be determined by the master dialogue file 232. If the master dialogue file 232 is subsequently changed so that these choices respectively become “Absolutely” and “No way,” these changes are automatically reflected in any conversation where the skin dialogue file 260 has null entries at these points. For the most part, however, the skin dialogue files 260 determine the response text, and the skin dialogue files 260 often overwrite the default responses of the master dialogue file 232.

Every skeleton dialogue file 234 has paired with it a You Decide How (YDH) skin dialogue file 260 that is designed in a broad, general way depending on the scope of the conversation determined by the skeleton dialogue file 234. For example, if a savoring skeleton dialogue file 234 is built to help the user savor a positive feeling, the YDH skin dialogue file 260 can determine all the sentences and phrases for this conversation. However, a new skin dialogue file 260 may be created from a base skin that focuses the user specifically on savoring food. A different skin dialogue file 260 may be created from this YDH skin 260 that focuses the user specifically on savoring an experience. Notably, due to the tiered architecture of the dialogue management system 230, no changes are required at the master 232 or skeleton 234 level to add this new activity. The only edits needed are to the YDH skin dialogue file 260, where any new phrases or guidance specific to food (or experience) can be added or edited. This new skin dialogue file 260 can then be paired with the savoring skeleton 234 to run a food (or experience) savoring conversation. Due to the tiered architecture of the dialogue management system 230, this versatility is accomplished without requiring code changes at the master 232 or skeleton 234 level. This significantly simplifies the design of the dialogue management system 230.

The master dialogue file 232 can offer a broadly-defined capability to identify an object of the conversation. The master dialogue file 232 includes the built-in architecture (CHTML, based data structures) to receive variables that can decide how the object is identified, how many questions are asked of the user, whether or not to provide a response at certain points, etc. The skeleton dialogue file 234 is where the flow-determining variables that are fed to the master dialogue file 232 are defined. Accordingly, the result of designing a skeleton dialogue file 234 is the decision to use the identify capability to ask two questions, for example, and respond any time the user identifies an emotion or an activity 244 based experience. The skin dialogue file 260 paired with the skeleton dialogue file 234 defines, among all of the dialogue’s specific text, the questions that can be asked, which for one particular skin dialogue file 260 may be “What is your favorite hobby?” and “How do you feel when you are engaging with this hobby?”. The skin dialogue file 260 paired with the skeleton dialogue file 234 additionally defines the full set of potential responses to emotions that might be provided in the answer by the user.

The master dialogue file 232 includes a library of sections or dialogue portions, each of which is a subset (or sub-dialogue) of a conversation that is focused on a single task 246 and includes distinct pieces of a conversation designed to achieve a goal in the conversation. Only a few of the dialogue portions are used during a dialogue. Further, some of the same dialogue portions may be used in combination with other dialogue portions in another dialogue. Essentially, for conducting a dialogue about an activity 244, a few of the dialogue portions from the master dialogue file 232, a skeleton dialogue file 234 corresponding to the activity 244, and a plurality of skin dialogue files 260 corresponding to the tasks 246 associated with the activity 244 are compiled together.

The dialogue management system 230 conducts the dialogue with the user in a versatile, life-like manner using the compiled combination of the dialogue portions from the master dialogue file 232, the skeleton dialogue file 234, and the skin dialogue files 260. This method of conducting dialogues eliminates the need to have a one to one correspondence between the number of dialogue portions of the master dialogue file 232 and the number of activities 244. For example, the dialogue management system 230 may include only a few sections, 10-20, about 60 activities and a much greater number of tasks 260. Accordingly, this method, comprising generic, modular, and reusable data structures designed in the master file 232, which are then selected by the skeleton 234 and modified by the skins 260, results in significant improvements and optimizations in the architecture and resource utilization of the databases of the app 200.

In a conversation, i.e., in a dialogue, a node is an atomic element. A node typically includes a prompt for the user and includes logic to process the user’s response to the prompt. The prompt and the user’s response (user input) can include one or more of text, speech/audio, and video including virtual reality (which can be used to extract body posture/positions facial expressions etc. for use as user input). Based on the processing of the response, the conversation moves to a next node. A section or dialogue portion in the master file 232 includes a group of nodes.

There are two primary types of sections in the master file 232: linear (or sequential) sections and adherence sections. The nodes in the sequential sections may be processed sequentially, i.e., a next node is processed when a condition is satisfied after processing a prior node. In an adherence section, after a node is processed, control always returns to the first node, and a check is performed as to which, if any, variable remains to be filled, and control moves to that node for which a variable needs a response. The process is repeated until all the variable are filled or until a counter expires. In case of a non-ending loop, e.g., due to repeated irrelevant responses from the user, a counter is maintained, and the loop is exited on expiration of the counter. The counter is only an example; instead, any other stopping condition that is guaranteed to be met within a reasonable number of conversation turns can be used.

Across the different sections or dialogue portions of the master 232, while the prompts may be different, and the content of the text (in the user response) may be different, the structure of the sections may be kept fairly steady across different activities 244. For example, in a conversation, regardless of the activity 244, the dialogue may start with a greeting and may end with a summary, both of which can be short, repeatable, i.e., reusable sequential sections. The dialogue may additionally include an adherence section to elicit responses for a few variables needed to conduct the dialogue. The dialogue may further include another section to clarify or disambiguate an item, for example.

These sections tend to have similar structures though different content. Further, irrespective of the number of activities 244 offered by the app 200, these sections of the master file 232 are few in number, i.e., they are not as many in number as the number of activities 244; or there is no one to one correspondence between the sections of the master file 232 and the activities 244. Accordingly, the master file 232 includes only a handful of sections and is a collection or an array of a few sections that (can but) do not include any specific content, e.g., what to ask, but have variables with generic values that can be and are usually overwritten by the skeleton 234 and the skins 260.

The skeleton file 234 simply contains a series of include calls that select a few sections (dialogue portions) from the master file 232 to accomplish the dialogue at hand. At this point, however, the dialogue management system 230 does not know the exact nature of the dialogue, e.g., whether the user wants to savor an experience or food. The skeleton 234 therefore also includes an identify section from the master file 232, which is very generic in nature, e.g., it can identify a person, an object, etc..

The values for the variables in these sections are provided by the skin file 260. These values are elicited from the user by the skin 260 by prompting the user with questions, e.g., multiple choice questions. The YDH skin file 260 is also general in nature, e.g., it can indicate savoring something but cannot further specify an experience or food. The task skin 260 provides the specific values for the variables that override the generic values of variables as well as specific values provided by the master file 232, if any. These features of the master file 232, the skeleton files 234, and the skin files 260 eliminate the need for providing custom dialogue scripts by anticipating every input from users, which again greatly simplifies the design of the dialogue management system 230.

The specific features or data structures employed by the master 232, the skeletons 234, and the skins 260 are now described. Throughout the remainder of the disclosure, while references are made to natural language classifiers (NLCs) and associated variables and values, NLC is used only as an illustrative and non-limiting example of a task performed by language modeling and analysis algorithms mentioned above.

The master dialogue file 232 includes the following features or data structures that are implemented in markup language or scripting language: conditional values, default NLC values, and a single array. In the conditional values features or data structures, as part of a variable/value pair, a capability to assign values based on a condition is provided, e.g., response_text can be assigned to a string based on the value of_ emotion. For the first condition that evaluates as true, the variable assignment is made, and no further conditions are evaluated. Unless defined, by default the “else” condition is equal to the current value of the variable, e.g., in the above example, the “else” value can be “ _response _text”.

In the default NLC values features or data structures, as part of the initial attributes of a section within the Script, included is an attribute named “nlc_defaults” which specifies what the output of a classifier should be depending on whether a classifier is used or not. Each classifier used in a section (dialogue portion) is identified by name and a default value is defined. If a classifier is present in a section (dialogue portion) and a default is not defined under nlc_default, the default value is a blank string.

In the single array of variables feature or data structure, for each choice within a single (or multi) input request, three attributes are defined: a “label”, an “Lgv_value”, and a “prompt”, with each choice identified by a “name” to the left of the colon, and the three attributes as strings defined to the right of the colon. The first attribute, “label”, is the text that should be presented as a choice to the user. The following two attributes are accessible as attributes of sensor objects after a selection is made. Accordingly, an Lgv _value(sensor) is an Lgv _value text of a choice that is made, and a prompt(sensor) is a prompt text of the choice that is made. In other words, to illustrate, if a user choses a third option, for example, Lgv value(sensor)==`third choice text’ and prompt(sensor)==`Response to third choice'. If the “label” of a choice is blank, then that choice is not presented. If every choice has a blank label, a validation error should occur (however, this happens at the level of the skeleton 234 and skin 260; the master 232 allows for all blank values that should be filled in at the skeleton/skin level).

The skeleton dialogue file 234 may contain “include” calls for selected dialogue portions from the master dialogue file 232, including both variable folders, global handlers, and sections (dialogue portions). The following feature or data structure may be implemented for the skeletons: NLC Switches, Variable Assignments, and Section-to-Section Flow. In the NLC switches features or data structures, as an attribute of an included section (dialogue portion) in the master 232, “nlc_active” defines whether a classifier is run or not in that section (dialogue portion). The “nlc_active” attribute defined in the skeleton works in conjunction with the “nlc default” attribute defined in the master dialogue file 232. When “nlc_active” for a classifier is set to false, the output of the classifier is the default defined in “nlc_default”. By default, each classifier present in an included section (dialogue portion) has an “nlc_active” value of false. So unless the skeleton dialogue file 234 defines an NLC as active (set to true), that classifier will not run in this section (dialogue portion).

In the variable assignments features or data structures, as an attribute of an included section (dialogue portion), “assign” redefines values for certain variables found in that section (dialogue portion). For any variable present in the section (dialogue portion) and not included in the “assign” list, the value remains as it is defined by the master dialogue file 232. However, the “assign” values made by the skeleton dialogue file 234 override the values set by the master dialogue file 232. Functionally, the assign values help define the flow and structure of an included section (dialogue portion), allowing importing a single block of code that can be used differently depending on the value of these variables. This feature is not merely better code but rather a better data structure architecture that yields efficiencies in database design and resource usage and significantly improves the functioning of the databases as one skilled in the art can appreciate.

The section-to-section flow feature or data structure is as follows. The master dialogue file 232 has “next″/“goto” statements that reference every section, i.e., dialogue portion within the master dialogue file 232. When a skeleton dialogue file 234 inclu des only a subset of the sections (dialogue portions) from the master dialogue file 232, references to those sections (dialogue portions) that are not included in the skeleton dialogue file 234 need to be handled. The master dialogue file 232 includes three “identify” sections (dialogue portions) named “identify”, “2nd_identify”, and “3rd_identify”. For example, a given skeleton dialogue file 234 may include only the “identify” and “2nd_identify” sections (dialogue portions). In the “2nd_identify” section (dialogue portion), the master dialogue file 232 has “next″/“goto” statements pointing to “3rd_identify”, which does not exist in this skeleton dialogue file 234 in this example. At runtime, this skeleton dialogue file 234 should simply move to the identified section (dialogue portion) in the master dialogue file 232 (the “3rd_identify” section or dialogue portion in this example) and then look sequentially section by section for the next section or dialogue portion that the skeleton dialogue file 234 actually does include.

In the skin dialogue files 260, there may be two levels of skins. A YDH (or overview) skin, and a task skin. The skin dialogue file 260 can be in a spreadsheet format but can ultimately run as a comma separated value (CSV) file in the content management system (CMS) 202 of the app 200. First few top rows under the headers rename the life graph variables (LGVs) used by the skeleton dialogue file 234. For every instance of the LGV name in the “Original” column, it is replaced with the name in the “Value” column across the entire skeleton dialogue file 234. If an LGV in the skeleton dialogue file 234 is either not referenced here or has a blank value in the “Value” column, the original name persists. Subsequent rows redefine the text of the skeleton dialogue file 234. The text in the “Original” column is a reference to the text in the master dialogue file 232 at that location. The “Value” column is the new text that replaces the existing text from the master dialogue file 232. If the “Value” column is blank, the value from the master dialogue file 232 persists. But the priority is given to the skin 260. Ideally, the YDH skin 260 can be automatically generated from a skeleton dialogue file 234 in the CMS 202 by identifying every LGV and every segment of text. An exported skin created by the CMS 202 would have an empty “Value” column. An “Author” column designates whether or not this row is to be included in an automatically generated task skin 260. A “0” indicates it is not included, and a “1” indicates that it is included.

The task skin 260 can be automatically generated from the YDH skin 260 by: (1) removing the rows with “Author” designated as “0” and then removing the “Author” column altogether; (2) assigning each “Value” entry of the task skin 260 as the “Value” entry of the YDH skin 260 if it’s not empty or the “Original” entry of the YDH skin 260 if the “Value” entry is empty; (3) creating an empty “Value” column; and (4) adding a “Legacy” column with one cell automatically populated with the “Short text”, “Description text”, and “Short text labels” already in the CMS 202 for the designated task 246. For each of these legacy task attributes, a tag is present that defines and separates the different strings. The “Value” column can then be filled in. When the CMS 202 is running an activity 244 using a task skin 260, it first prioritizes the “Value” entries from the task skin 260; if those are empty, next prioritizes the “Value” entries from the YDH skin 260; and if those are also empty, lastly prioritizes the “Original” entries from the YDH skin 260. If all of these values are blank for an “ask″/“prompt” or “next″/“text” entry, the dialogue management system 230 does not create a text bubble and continues with the flow of the dialogue. As described above, if the value for a single/multi label is blank, then it is not shown, and if all the labels for a single/multi input are blank, there is a validation error. The task skin file 260 is still paired with the original skeleton dialogue file 234. Accordingly, for example, to run S-01 Savor the Small Stuff in “You Decide How” mode, the dialogue management system 230 pairs the S-01 skeleton dialogue file 234 with the S-01 YDH skin file 260; to run S-01-T-27 Smell the Roses, the dialogue management system 230 pairs the S-01 skeleton dialogue file 234 with the S-01-T-27 task skin file 260; and so on.

In FIG. 5B, the user initiates the dialogue 270, e.g., using a drop down menu from the app 200, which is presented on the user’s device, e.g., client device 120-1 in the form a user interface (UI). For example, the dialogue box 270 can appear similar to the UI of a text messaging app on a smartphone. In the dialogue 270, the entity “Service” represents an automated conversational agent driven by the 3-tier architecture of the dialogue management system 230 described above.

The dialogue 270 can begin with a greeting. The dialogue 270 can end with a summary and/or another greeting. The dialogue 270 provides the app 200 (via the dialogue management system 230) another opportunity, in addition to the tracks 242, activities 244, and tasks 246, to effect an intervention, for example, by coaching the user on a particular happiness skill such as how to practice empathy or how to improve practicing empathy. The dialogue 270 also offers the user the opportunity to share his or her experience, exhibit his or her skill level regarding a particular happiness skill via the dialogue 270, and improve the particular happiness skill based on the coaching received from the app 200 via the dialogue 270.

While not shown, the dialogue 270 can include text message as well as audio/video messages from either or both of the service and the user. Further, the dialogue can also include graphics such as emoticons, photos, videos, music, and so on that can be exchanged by and between the service and the user, i.e., either or both of the service and the user can also provide the graphics such as emoticons, photos, videos, music, and so on.

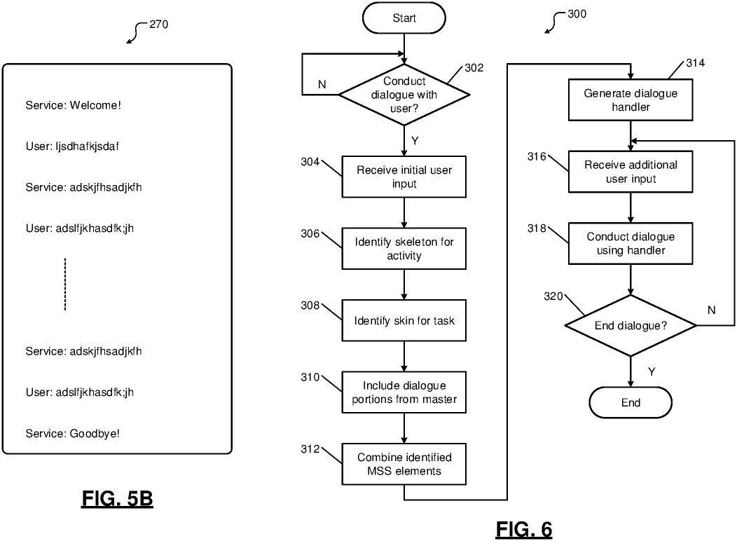

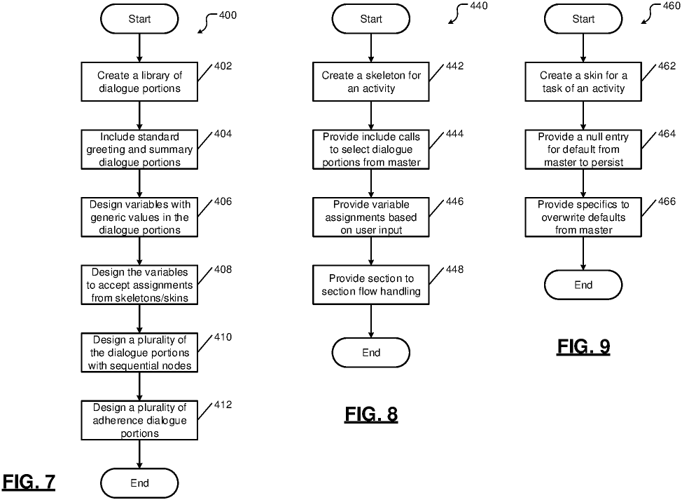

FIG. 6 shows a method 300 for conducting a dialogue between the app 200 and a user of the app 200 using the dialogue management system 230. For example, the method 300 is performed on one of the servers 130 and includes presenting the dialogue 270 on a user device such as the client device 120-1 via the distributed communications system 110.

At 302, the method 300 checks whether a user is initiating a dialogue 270 with the app 200. At 304, if a user initiates a dialogue 270 with the app 200, the method 300 receives an initial input from the user. At 306, based on the user input, the method 300 determines an activity 244 that the user wants to discuss in the dialogue 270 and identifies a skeleton file 234 for the activity 244. At 308, the method 300 identifies a skin file 260 for a task 246 associated with the activity 244. At 310, the method 300 includes dialogue portions from the master file 232 selected based on the activity 244 to conduct the dialogue 270. At 312, the method 300 combines the selected dialogue portions of the master file 232, the skeleton file 234 for the activity 244, and the skin file(s) 260 for the task 246, e.g., the method 300 compiles these master 232, skeleton 234, and skin 260 elements. At 314, the method 300 generates a dialogue handler generated based on the combination or compilation that is used to conduct the remainder of the dialogue 270.

At 316, the method 300 receives additional inputs from the user. At 318, the method 300 conducts the dialogue 270 with the user based on the user inputs using the dialogue handler, e.g., the method 300 interactively responds to the user inputs. At 320, the method 320 determines if the user wants to end the dialogue 270. The method returns to 316 if the user wants to continue the dialogue 270. Otherwise, the method 300 ends.

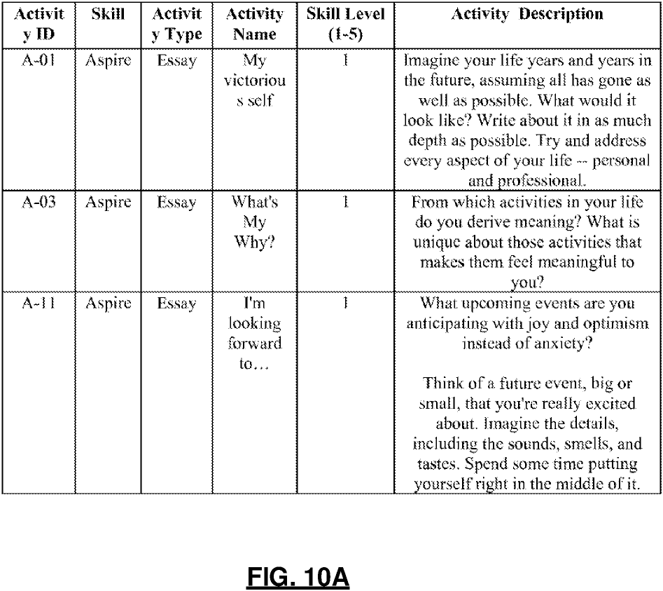

FIG. 7 shows a method 400 for designing and generating the master file 232. At 402, the method 400 creates a library of dialogue portions such that the number of dialogue portions is less than the number of activities 244, i.e., there is no one to one correspondence between the number of dialogue portions of the master file 232 and the number of activities 244 offered by the app 200. For example, the method 400 identifies and takes advantage of any overlap or redundancies across the activities 244 offered by the app 200.

At 404, in the library of dialogue portions, the method 400 creates a standard greeting dialogue portion to be presented at the beginning of any dialogue 270 irrespective of underlying activity 244, and a standard summary dialogue portion (or another standard greeting dialogue portion) to be presented at the conclusion of any dialogue 270 irrespective of underlying activity 244. At 406, the method 400 designs variables with generic values (and a few variables with specific values) in the dialogue portions of the master file 232. At 408, the method 400 designs or configures the generic variables to accept specific value assignment from skeletons 234 and skins 260. At 410, the method 400 designs a plurality of the dialogue portions of the master file 232 to include sequential nodes. At 412, the method 400 designs or configures a plurality of the dialogue portions of the master file 232 to function or operate as adherence dialogue portions.

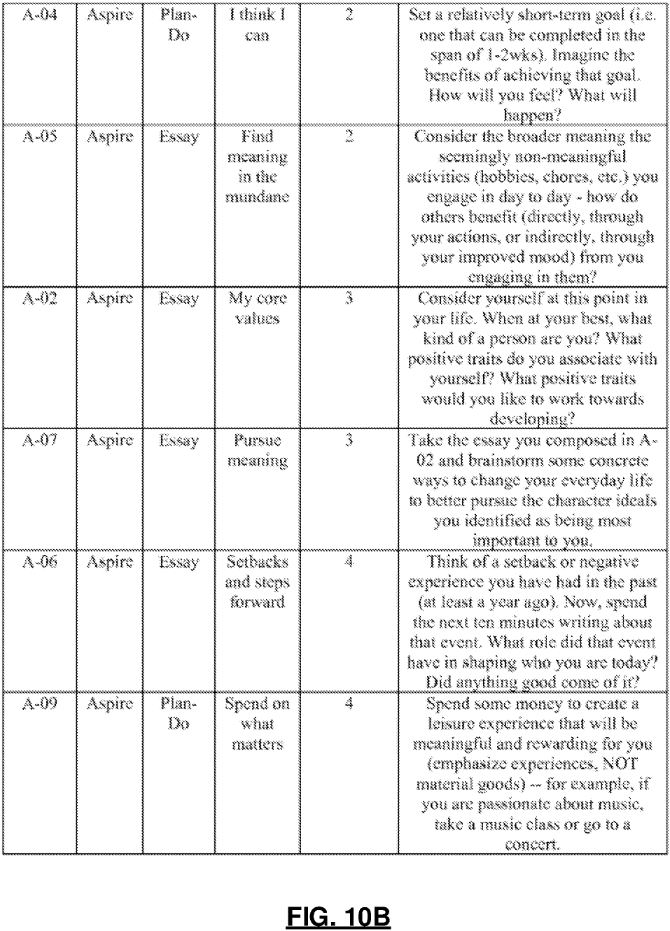

FIG. 8 shows a method 440 for designing and generating skeleton files 234. At 442, the method 440 creates a skeleton file 234 for an activity 244, i.e., the method 440 creates one skeleton file 234 per activity 244 offered by the app 200. At 444, the method 440 provides “include” calls in the skeleton file 234 to select relevant dialogue portions from the master file 232. At 446, the method 440 provides variable assignments to the selected dialogue portions based on user input to conduct the dialogue between the user and the app 200. At 448, the method 440 provides section to section flow handling to conduct the dialogue between the user and the app 200. For example, the order in which the flow of or between the sections is conducted during a dialogue may be different than the order in which the sections are arranged in the master file 232.

FIG. 9 shows a method 460 for creating a skin file 260. At 462, the method 460 creates a skin file 260 for a task 246 for an activity 244, i.e., the method 460 creates a skin file 260 for each task 246 of an activity 244 offered by the app 200. At 464, the method 460 provides an indicator such as a null entry to allow for a default value for a variable from the master file 232 to persist. At 466, the method 460 provides a specific value to overwrite a default value for a variable from the master file 232. The specific value is based on the user input and is passed to the skeleton file 234, which then assigns it to a suitable variable in a selected dialogue portion from the master file 232.

The dialogue management system 230 of the present disclosure differs from a chatbot. A chatbot is a very general description of any conversational agent that communicates with a user via text or voice/video on a turn by turn basis. A chatbot can therefore be intelligent, e.g., use machine learning or completely pre-scripted; so it is very broad in scope. The differences between the dialogue management system 230 of the present disclosure and a chatbot are in the specific applications and its 3-tier architecture based on the specific applications. The dialogue management system 230 does not focus on delivering efficacious psychological interventions in the best possible way, and on using machine learning and dialogue management mechanisms to accomplish that. Rather, the dialogue management system 230 is an efficient way to create and program a “chatbot” using the 3-tier architecture described above so that the scripts governing the dialogues do not have to be created for all possible conversational scenarios and so that the scripts governing the dialogues can reuse some code.

Further, the dialogue management system 230 of the present disclosure differs from other automated customer support systems. Specifically, the difference is due to the operation of the dialogue management system 230 based on the tracks 242, the activities 244, and the tasks 246, where the activities 244, about which dialogues are conducted, are recommended by the app 200. This schema of the app 200 creates a unique opportunity for designing the synergistic 3-tier architecture to conduct dialogues as described above. Unlike the app 200, systems that do not evaluate feedback from users regarding activities recommended by the systems and that do not attempt to improve user behavior via interventions offered based on the feedback, naturally lack the need for the 3-tier architecture described above. Of course, the dialogue management system 230 can be used with any other system that evaluates feedback from users regarding activities recommended by the system and that attempts to improve user behavior via interventions offered based on the feedback.

In sum, the dialogue management system 230 of the present disclosure uses a novel 3 layer approach—a master file 232 that can cater to dialogues on any of the nearly 60 activities offered by the app 200, a skeleton file 234 that is specific per activity 244 and that links to one or more “sections” or dialogue portions in the master file 232 (some of which can be reused for another activity 244), and a plurality of skin files 260 that handles the input and output at the user interface presented to the user as a dialogue box 270. For each dialogue 270, these 3 elements are combined and a dialogue 270 is conducted. For another user or another activity 244, another combination is used to conduct another dialogue 270. The synergy provided by the 3 tier approach is that the generic nature of the master file 232, the ability of the skeleton file 234 to include sections of the master file 232 in any combination as needed, and the ability of the skins 260 to provide the specific values to variables in the selected sections of the master file 232 result in significant reuse of the sections of the master file 232, which yields efficiencies in database design and use of database resources. The dialogue management system 230 is versatile in that it works across all activities 244 offered by the app 200 and regardless of the variations in the user’s inputs and in the activities 244. Thus, the 3 tier design of the dialogue management system 230 improves the functionality of the computer databases 206, not merely code.

Spatial and functional relationships between elements (for example, between modules, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

In certain embodiments of the present invention, the app 200 embodies a service of various treatment and prevention disciplines, such as positive psychology, cognitive behavioral therapy, mindfulness, stress reduction, etc. One exemplary service is referred to herein for convenience as “Happify.” Happify is a novel, science-based app for engaging, learning and training the skills of happiness. Happify is based on a framework developed by psychologists and researchers in a collection of therapeutic disciplines such as Cognitive Behavioral Therapy, Mindfulness, positive psychology etc., and assists users in the development of certain skills related to being happy, for example, Savor, Thank, Aspire, Give and Empathize (or S.T.A.G.E.^{™}). In certain embodiments, each skill is developed using various activities, ordered in increasing skill level, that gradually unlock as the user progresses in building that skill. With Happify, a user selects a “track” that contains sets of activities that are designed to address a specific life situation or goal.

As the user performs one or more of these activities, the Happify system assesses and re-assesses the user’s physical and emotional states using various tools. For instance, there may be a plurality of sensors, e.g., biometric that are placed within a vicinity of the user, e.g., in wired and/or wireless communication with the user’s smartphone that extract biometric information from the user while the user is engaged in an activity or a task. Examples of such extracted biometric information are heart rates, heart rate variability, brainwaves, body heat, pupil dilations, etc. In another instance, one or more sensor mechanisms within the user’s smartphone, e.g., speaker, camera, microphone, buttons, keys, etc. are used to capture user information. Examples of such captured information are recorded speech, typed texts, facial expression, etc. In a further instance, the user’s physical or emotional states may be assessed from self-reports such as questionnaires. In other instances, a mix of foregoing information may be used concurrently to assess the user’s physical or emotional states.

In accordance with the Happify system, the extracted, captured and/or otherwise provided information are processed to analyze the user’s feelings including, but not limited to, the user’s reaction, the user’s engagement level, the user’s adherence level, the change in the user’s psychological state, etc. in regards to the performed, or partially performed, Happify activities. Processing may be carried out within the Happify application or by another processing unit that resides within the smartphone (or tablet or other computing system). Alternatively, the extracted and/or captured information are transmitted and processed remotely by a server (or other remote electronic device). In any of these versions, processing includes application of select mathematical algorithms and analytical computations on user input data obtained while the user performs the activities. The processing ultimately results in providing of select follow up activities that further enhance development of the happiness skill in order to achieve the desired outcome.

In further accordance with the Happify system, the processing of data and/or the providing of follow-up activities is ongoing. In particular, as the user performs the provided activity, the Happify system continually monitors and interacts with the user to obtain ongoing real-time information. For example, the ongoing real-time information may be a user’s response to a question, what the user has done in response to a task, or various other biometric information of the user obtained from the sensor(s) placed within a vicinity of the user. With such real-time or aggregate analysis, the user’s interaction with the Happify system becomes more dynamic and results in higher levels of engagement as that interaction continues.

Further details of the Happify system and operation of the Happify system are set forth in U.S. Pat. Application Ser. No. 14/284,229, entitled “SYSTEMS AND METHODS FOR PROVIDING ON-LINE SERVICES” and U.S. Patent Application Ser. No. 14/990,380, entitled “DYNAMIC INTERACTION SYSTEM AND METHOD,” and the entire contents of each of these applications is incorporated herein by reference. For the sake of brevity, further details of the Happify system/service are not provided herein (except as otherwise described herein).

### Empathy

In accordance with the present invention, the computing system further dynamically responds to the user’s actions and feedback by demonstrating simulated human emotion and/or human cognitive skill. In certain embodiments to be discussed, the computing system is configured to demonstrate empathy.

In further accordance with the present invention, a computing system is equipped or otherwise programmed with artificial intelligence for simulating a variety of human emotion and cognitive functions. For purposes herein, the term artificial intelligence (AI) means a machine or device suitably adapted or programmed in a manner sufficient so that the machine or device perceives its environment (or the desired environment) and takes actions that maximize its chance of successfully achieving its intended goals, as well as processes carried out by such machines or devices. The term AI can further mean the ability to learn from data and generalize unseen data by a machine. Display of artificial intelligence by a computing system generally includes performance of tasks that normally require a human intelligence. Various embodiments of the present invention are directed to demonstration of artificial “emotional” intelligence, which is a particular subset of human intelligence.

The field of artificial intelligence draws upon various diverse fields, such as computer science, mathematics, psychology, linguistics, philosophy and many others. In more recent years, AI has progressed to the point of understanding (at least from the machine’s perspective) the aspect of human intelligence that is known as emotional intelligence, e.g., empathy. The term “empathy” generally is defined as the (human) ability to understand and share the feelings of another. In other words, empathy is the capacity to understand or feel what another person is experiencing from within the frame of reference of the other person. With recently developed AI emotion models, machines can now be programmed to learn when and how to display emotion in ways that enable the machine to appear empathetic or otherwise emotionally intelligent.

In accordance with the present invention, the above discussed Happify system further interacts and engages with users in an empathetic and supportive manner to provide certain benefits as herein described. The system/process of the present invention, therefore, in certain embodiments, is capable of emotional intelligence and with such emotion intelligence, conveys empathy to users of the system to keep the user advantageously engaged over time.

### Mirroring Prompt

In certain embodiments, the inventive system includes artificial intelligence sufficient to provide the system with a so-called “mirroring” ability. As described herein, the inventive system in such certain embodiments employs various algorithms, such as topic analysis, natural language classification, etc. to reflect back on input received from the user and/or measurement data collected from the user, and then responds to the user with context-based responses.

In each of the embodiments described herein in which AI is employed by the inventive computing system to convey or simulate emotional intelligence, the environment presented to the user beneficially is human-like from the perspective of the user that results in a more rewarding or engaging environment to the user that, in turn, results in greater engagement by the user that, in turn, results in a far greater chance of success in the ultimate goal of achieving a greater level of happiness.

In accordance with the present invention, the “next” step in the interaction may depend on what rules have been set in regards to the provided activity. For example, the mirroring stage may be performed in a loop until the computing system decides to move onto the next question to ask. As another example, the next step may be based on the user’s input. As a further example, the mirroring stage may be an interim stage that may be used at each “turn” of the interaction and the determination for the next turn may be based on adherence fidelity. Additional details of the adherence fidelity feature of the present invention is provided in the U.S. Provisional Application Ser. No. 62/533,423, filed on Jul. 17, 2017, the entire content of which is incorporated herein by reference.

The mechanism of mirroring entails maintaining the same flow of interaction with the user and including an appropriate “mirroring prompt” in the interaction. For example, when two people communicate, it has been scientifically researched that their brains tend to get activated in similar regions. This effect is also known as “brain mirroring.” See “Brain Basis of Human Social Interaction: From Concepts to Brain Imaging” by Hari, R., & Kujala, M. V., Physiological Reviews, 89(2), 453-479 (2009) for additional detail on brain mirroring, the content of which is incorporated herein by reference.

In accordance with an exemplary computing system of the present invention, the anatomy of a mirroring prompt can be outlined as follows: (1) Reflecting the content of what the user just said; (2) Using an understanding and supportive tone; (3) Using an emotional tone that is similar to the emotions the user conveyed or an emotional tone that is appropriate for the emotions the user conveyed; and (4) Addressing the context or situation that the user mentioned. The mirroring prompt demonstrates that the computing system “feels” what the user is feeling and, naturally, responds in a similar tone, mirrors the content of the conversation, conveys commiseration, etc.

Without mirroring, the computing system jumps to providing the user with solutions. However, with mirroring, the system has employed a mirroring prompt using a similar tone to reflect back “going back to school” and “feeling drained,” while mentioning that “everyone” feels drained from time to time, thus showing that it understands how the user is feeling. Again, similar to the first example, the user feels more appreciated and engaged with the conversation when mirroring is implemented.

As such, to better identify and understand the contents of the conversation, the computing system employs a set of techniques such as natural language classification, topic modeling, sentiment analysis, named entity extraction, emotion detection, etc. The list is not exhaustive and the computing system may employ additional techniques as necessary to identify and understand a broad spectrum of topics. The series of steps in applying various analytic techniques is also referred to herein as the computing system training a “classifier.”

Various details of topic or language modeling techniques that may be employed in certain embodiments of the present invention are not described, but rather are sufficiently and well understood in the art. Those details that are well known and understood are not described herein for brevity. Various publications that describe such techniques that may be employed herein include: “Probabilistic Topic Models” by Blei, D. M., Communications of the ACM, 55(4), 77-84, (2012); “Utopian: User-Driven Topic Modeling based on Interactive Nonnegative Matrix Factorization” by Choo, J., Lee, C., Reddy, C. K., & Park, H., IEEE Transactions on Visualization and Computer Graphics (Volume: 19, Issue: 12, December 2013); and “Hierarchical Topic Models and the Nested Chinese Restaurant Process” by Griffiths, T. L., Jordan, M. I., Tenenbaum, J. B., & Blei, D. M., Published in NIPS′03 Proceedings of the 16th International Conference on Neural Information Processing Systems, Pages 17-24 (Dec. 9-11, 2003), and each of these publications is incorporated fully herein by reference.

Next, the computing system runs additional clustering analyses to group together various themes and topics. For instance, this may require further grouping together themes and topics that may be facially different but nonetheless require a similar response to the user. For example, “working in the yard” and “being outdoors” may be grouped together as the mirroring prompt would be the same, e.g., “being outdoor is great!” regardless of whether the user is describing his or her effort in mowing the lawn or taking a leisurely walk in a park. Still further, this is particularly effective if the same response for different topics has the same psychological effect, as at the end of the day, the goal is to cater to the efficacy of a psychological intervention.

Once the reference data has been grouped into major themes via the steps described above, the computing system identifies the most representative text sample of the theme. The most representative text sample may be determined by scoring each text sample to assess its proximity or degree of match to each topic, and then using only the samples with the closest match (or top-scoring) as the most representative. Using these data, a text classifier is trained that can learn to distinguish between themes. For example, the text classifier can use features extracted from the text such as the topic scores or other language model scores, e.g., word2vec scores, and then use another classification algorithm, e.g., Bayesian classifier, support vector machine, deep learning, neural network, etc. to learn to distinguish between the features. In a case where voice or video data are used, the computing system may further include AN classification algorithms, such that the content beyond the text, such as the tone of the voice or the facial expression may also be used.

Some of the classification algorithms that are discussed above as being utilized by the Happify system are also known in the art. Details of the specifics of the known algorithms are omitted herein for brevity. Instead, below list demonstrates exemplary publications that are incorporated herein by reference that describe respective exemplary algorithms: “A Comparison of Event Models for Naive Bayes Text Classification” AAAI-98 Workshop on Learning for Text Categorization (Vol. 752, No. 1, pp. 41-48); and “word2vec Explained: deriving Mikolov et al.'s negative-sampling word-embedding method” Goldberg, Y., & Levy, 0., arXiv: 1402.3722 (2014).

After identifying and understanding the contents of the conversation, and before responding to the user demonstrating the understanding of the content of the user’s statements, the computing system must detect the “tone” of the user’s statements and respond using an emotional tone that is similar to or appropriate for the tone the user has conveyed. In particular, understanding and emulating the user’s tone allows the computing system to demonstrate that it is aware of the user’s feeling toward what is said and that understanding makes it feel the same feeling.

As an alternative to choosing from a list of available prompts, once the user’s tone or other characteristic has been identified, the computing system may synthesize a new prompt using natural language generation techniques. For example, using the entity “John,” the relationship “brother,” the topic “meal,” the subtopic “dinner” and the tone “fun,” the computing system may synthesize “Sounds like your brother John and you had a fun time during dinner!” As a further alternative, the computing system may draw from an inspirational quote or mention a fact from a research study. In some versions, the prompt may also be composed using real time query of online resources. For example, the prompt can be based on the variety of information that is available on the web. If it is detected that the user is describing a topic that happened recently, the computing system can go online to news websites and generate a prompt taking these events into account. In accordance with the present invention, generating a prompt with information that is based on recent event may be more effective in grabbing the user’s attention. For instance, if the name of a rock band is continuously detected as a topic, providing a real time update on that rock band may serve to draw the user deeper into the conversation. Once the mirroring prompt is administered and played to the user, the computing system continues with the normal course of interaction with the user.

In accordance with the present invention, if and when the mirroring prompt feature is activated, a sub component such as a dialogue manager or an interaction manager within the computing system may perform one or more of the analyses discussed above. Various components may work concurrently to train and/or retrain the classifier in real time, run real time analysis on the dialogue or the conversation, and retrieve or generate a mirroring prompt that serves multiple purposes, e.g., show empathy, increase adherence, etc.

In certain embodiments, an interactive session as discussed above is defined by the user freely speaking in the presence of the computing system. During the interactive session, the computing system may similarly speak back to the user and engage in an auditory conversation with the user. The computing system may intelligently adjust volume, pitch, gender, etc. of the spoken voice to as part of simulating empathy. For example, the computing system may distinguish a loud voice response from a quiet voice response. The computing system may also distinguish a rapidly spoken response from a calmly spoken response. The computing system may further distinguish an immediate response from a contemplated response. As such, the mirroring prompt may be more verbose or succinct or more high-key or low-key. When it is detected that the user is taking his or her time to answer a question, even prior to receiving a response, the computing system may ask what the user is thinking about. Accordingly, the mirroring prompt is not only relevant and indicative of identified topics and/or reflective of the ascertained tone from the user’s response, but also contemplative of the user’s mood, the user’s habit, the user’s manner, the user’s style, etc.

### Types of Interactive Sessions

An interactive session is triggered when the user is presented with an activity to be performed. As described above, some exemplary activities require the user to answer a series of questions. When these types of activities are presented, the session may become “interactive” when the user provides a response. As discussed above, the inventive computing system analyzes the text of the received response and simulates conveyance of empathy to increase the user’s level of engagement to a particular activity or a happiness track.

In certain other embodiments, the user communicates with the computing system via a screen and a keyboard by ways of typing and reading words on the screen. The computing system may intelligently adjust the manner in which words are displayed, such as color, font or size or incorporate pictures or short video clips as part of simulating empathy.

In certain further embodiments, when a more physical activity is presented, such as requiring the user to perform a certain action, e.g., perform an exercise, go interact with other people, etc., the performance of the activity by the user is monitored via various modules and sensors in connection with the computing system. When these types of activities are presented, the session may become “interactive” upon the computing system detecting a certain facial expression or a certain bio-physical change. For example, when the user is instructed to perform a particular exercise to help clear the user’s mind, the computing system may monitor the user’s heart rate and interrupt to provide an alternate activity when the user’s heart rate has reached a certain threshold. Or, the computing system may monitor the user’s posture and provide a guiding prompt. In these embodiments, the computing system can also simulate empathy, just as it does in an auditory or a visual conversation, by expressing a mirroring prompt that shows an understanding of the user’s current feelings and/or by providing words of encouragement to show that the computing system is watching the user’s performance in the shoes of the user.

As another example, when the user is performing a physical action as part of performing the presented activity, the computing system may analyze the facial expression, the voice, the gestures, etc. of the user to determine the user’s mood or attitude toward the particular activity. Based on detecting certain facial expressions or hand gestures, the computing system may output a mirroring prompt. In accordance with the present invention, based on detected facial expression, the mirroring prompt may be commiserative, encouraging, sympathetic or mirroring. In other words, these additional input data from the sensors impact how the computing system determines the tone of the outputted mirroring prompt.

Accordingly, the feature of providing a mirroring prompt during an interactive session can be achieved through numerous ways. In the end, the computing system displays emotional intelligence by mirroring the user in the most appropriate way possible and such effect leads to a higher level of engagement and an increased commitment to remain engaged with the activity or track.

### Proactive Triaging

In certain other embodiments, the inventive system includes artificial intelligence sufficient to provide a “proactive triaging” ability. One of the biggest causes for a drop in the level of engagement with sustained usage of program or application such as Happify is that the user is not finding a particular activity exciting or relevant. There may be additional different reasons why a user may not find wish to further engage with an activity. In some cases, the user is partaking in an activity while internally desiring something else. Most of the time, the user would not even bother requesting for a change and simply lose interest in continuing with the program. In one or more of these cases, it may be that the user is simply preoccupied with a certain different issue without fully realizing it.

As described in greater detail herein, with such proactive triaging ability, the computing system is capable of detecting, during an activity in progress and/or during an interaction with a user, that the user is currently focusing on a topic other than the one intended by the system, or focusing on a topic that is more relevant to a different Happify track or activity, and in such case, the system “proactively” suggests a suitable change to the user. Discovering the fact that the user is preoccupied with a different issue is in fact a new insight and a realization shared with the user. For instance, during execution of a particular activity within a selected Happify track, the computing system detects particular user behavior, characteristics and/or user feedback indicating a necessity for proceeding with a different activity within the selected track or proceeding to a different Happify track entirely and recommends a change to the user when appropriate.

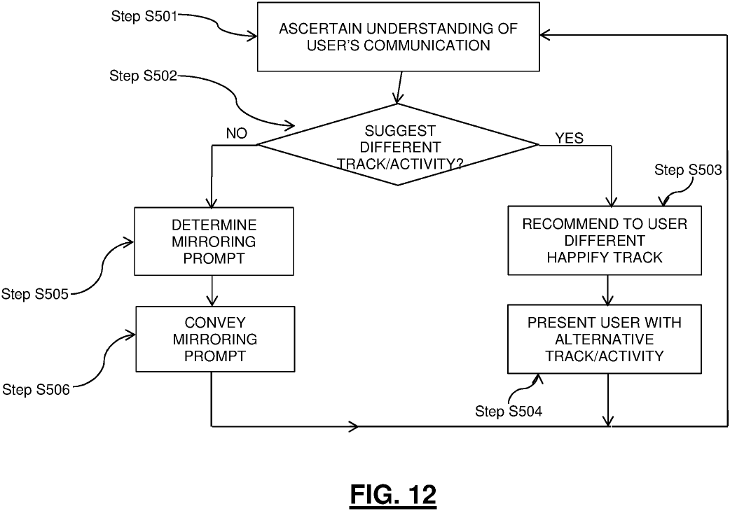

In accordance with an exemplary embodiment of the present invention, the user is engaged in an interactive session with the computing system. As shown in FIG. 12, the process for proactive triaging begins with ascertaining an understanding of the user’s communication (Step S501). This step, similar to the mirroring feature described herein, entails multiple sub-steps. For example, the computing system employs techniques such as natural language classification, topic modeling, sentiment analysis, named entity extraction, emotion detection, etc. to identify and understand the contents of the user’s communication. As previously described herein, the computing system may, for example, employ a trained classifier and identify one or more topics from the user’s communication.

Once the computing system identifies topics from the content of the user’s response, it determines whether a branching suggestion should be made (Step S502). This step also entails multiple sub-steps. For example, the computing system may employ a threshold system in which a determination as to suggesting a different track/activity is made when words suggestive of a different topic appear a certain number of times. As another example, the determination is made when none of the topics identified relates to the current activity/track. As yet another example, relevance of identified topics to the current activity/or track may be measured in a range of scale, and the branching determination is made when the relevance of the identified topics to the current activity/track is below a threshold level. As a further example, the computing system detects certain keywords that necessitate a branching suggestion. In some embodiments, the exact same set of Al engines as described above, e.g., emotion detection, topic modeling, natural language classification, etc. are used to determine whether or not the branching suggestion should be presented to the user. For example, sensors may detect certain facial expressions or gestures indicating lack or loss of interest and the computing system determines that the branching suggestion should be made. As another example, the computing system may keep a track of the progress of the user in regards to the provided activity and/or the selected Happiness track, and a branching determination is made based on the level of progress of the user. The goal of proactive triaging is that at each and every turn in the dialogue/conversation, the computing system conducts proactive triaging to re-evaluate what is the best course of interaction/treatment for the user.

If it is determined that the branching suggestion should be made, the process proceeds to step S503. In step S503, the computing system notifies the user that the user is seemed to be focusing on a topic that is different from the current activity and presents a recommendation. When the user accepts the suggestion, the computing system presents the user with alternative track/activity that has been determined as the better course of action for the user (Step S504). Thereafter, the process can be repeated to determine how well the user is interacting with the new activity/track.

If it is determined that the branching suggestion is not needed, the process proceeds to step S505. In Step S505, the computing system determines a mirroring prompt and in Step S506, the computing system conveys the mirroring prompt to the user.

In some embodiments, the proactive triaging feature is employed without the mirroring prompt feature. In certain other embodiments, the proactive triaging feature is employed concurrently with the mirroring prompt feature. In yet certain other embodiments, the mirroring prompt feature is carried out prior to the proactive triaging feature. Therefore, in some embodiments, the proactive triaging feature is the “next step” to the process of mirroring as disclosed herein. In accordance with the present invention, proactive triaging, thus, can be referred to as first, empathizing with the user and second, providing an advice or making a suggestion for a course of action to the user based on understanding of the user’s emotion. More particularly, with proactive triaging, the computing system analyzes, for example, what the user has said and the manner in which it is said and provides an appropriate suggestion. In some embodiments, the computing system will not only provide a suggestion, but also explain the reasoning behind it.

An example of the proactive triaging in a conversation employed by the present invention is shown in Tables 5 and 6.

TABLE 5

Steps Leading to Proactive Triaging

| | |
|---|---|
| Computer | > What are things you can do to improve the quality of_ the time you spend with your children? |
| User | >> Perhaps if I stopped thinking about my debt and the possibility of filing for bankruptcy when I am with them, perhaps then I could be more present.. |
| Computer | > Yes, it’s normal to worry about things, but too much worry can rob us of good life experiences. |
| Computer | > Is there something else you can do to improve the quality of the time you spend together |
| User | >> Well, I was thinking of taking them to the movies or even on a day trip up to the mountains, but every time I think of something like that I get scared about expenses and not being able to pay for it |

TABLE 6

Proactive Triaging

| | |
|---|---|
| Computer | > What are things you can do to improve the quality of the time you spend with your children? |
| User | >> Perhaps if I stopped thinking about my debt and the possibility of filing for bankruptcy when I am with them, perhaps then I could be more present. |
| Computer | > Yes, it’s normal to worry about things, but too much worry can rob us of good life experiences. |
| Computer | > Is there something else you can do to improve the quality of the time you spend together? |
| User | >> Well, I was thinking of taking them to the movies or even on a day trip up to the mountains, but every time I think of something like that I get scared about expenses and not being able to pay for it. |
| Computer | > I noticed that you mention worrying or perhaps being stressed about finances. Happify has a track called: “Dealing with Financial Stress.” Would it be useful for you to check it out? |

Initially, it should be noted from the above conversation that the computing system has employed the mirroring prompt and demonstrated human-like empathy by demonstrating an understanding tone and reflecting on the content of what the user just said, e.g., “it’s normal to worry about things”. Moreover, the computing system continues the interaction and receives the user’s further responses. During the course of the interaction, the computing system performs aforementioned analyses on the input data and identifies one or more words that are indicative of a different topic being mentioned repeatedly. For instance, in the above example, the computing system identifies the terms “debt,” “bankruptcy” and “expenses” that all belong to another group, e.g., “financial management”. The computing system also recognizes a negative tone in relation to the usage of these terms in the conversation. The computing system also recognizes a repetition of these terms in the conversation. At this point, as shown in Table 6, in addition to simply empathizing or showing support, the computing system proactively suggests that the user switch to a different track that is focused on financial worry.

The present digital therapeutic is designed to improve patient conditions according to one or more clinical measurements. For example, the Patient Health Questionaire-9 (PHQ-9), also called the DEP-9, is a depression scale from the Patient Health Questionaire (PHQ) containing nine questions that is used to make a depression diagnosis according to DSM-IV criteria. The PHQ-9 may also be used to track the progress of a user over time. Generalized Anxiety Disorder 7 (GAD-7) is similar to PHQ-9 but focuses on anxiety issues instead of depression and may be used similarly to diagnose and track anxiety. The digital therapeutic described herein creates physiological changes in patients that may be measured by the PHQ-9, GAD-7 and similar tools.

A number of implementations have been described. Nevertheless, it will be understood that various modifications may be made without departing from the spirit and scope of the invention. Accordingly, other implementations are within the scope of the following claims, including the following implementations, expressed as interrelated items:

### Digital Therapeutics for Migraine

The migraine digital therapeutic presented herein is a multi-week computerized behavioral therapy used to treat migraine, either directly or in connection with treating one or more mental disorder such as depression and anxiety. More specifically, the treated disorder may be one of more of major depressive disorder (MDD) and generalized anxiety disorder (GAD) and psychiatric disorders related thereto. The symptomology or comorbidity relationship between migraine and any psychiatric disorders may be indirect. That being said, treatment of disorders such as MDD and/or GAD have been shown to impact migraine symptoms or side effects of migraine treatments such as CGRP receptor antagonists.

The digital therapeutic presented here is based on the principles of cognitive behavioral therapy (CBT) and the related disciplines of acceptance and commitment therapy (ACT) and positive psychology. The digital therapeutic is intended to impact migraine symptoms and side effects by treating MDD and/or GAD. This MDD/GAD treatment is based upon correcting maladaptive patterns of cognition and helping patients engage in healthier behaviors.

### Generally Useful Definitions

Cognitive Techniques. These interventions emphasize making changes to maladaptive thinking patterns that maintain psychiatric conditions. Cognitive techniques might include: evaluating thoughts based on evidence, conducting experiments to gather new evidence to evaluate a thought, and thinking through the probability of negative outcomes. For example, cognitive techniques for MDD and/or GAD often include challenging unhelpful positive beliefs about worry, e.g., superstitious beliefs that worrying prevents negative outcomes).

Behavioral Techniques. Behavioral interventions are grounded in learning theory (operant and/or classical conditioning) and emphasize reward, punishment, habituation and extinction. Examples include exposure to feared stimuli or sensations to reduce anxiety and engagement in valued activities to reduce depression by increasing exposure to rewards.

Positive Psychology. Interventions from positive psychology include any skill to help patients notice pleasant experiences or emotions, focus on positive aspects of their lives, or improve functioning above baseline or statistical normality. Examples include gratitude exercises, imaging future events with joy and optimism, counting blessings, etc.

Psychoeducation. Psychoeducation provides important psychological information to the patient, typically about the disorder being treated or about the techniques that will be used within the treatment. In the digital therapeutic presented herein, we provide psychoeducation about functions of worry, mindfulness and acceptance as an alternative to worry, particularly in the context of migraines, how savoring and engagement in valued activities can be helpful, etc.

Daily Monitoring. This involves daily recordings of important symptoms or behaviors. In the present digital therapeutic product, a daily worry diary for much of the multi-week program may be included. It is designed to gather information about the patient’s worry patterns, e.g., triggers, common worry topics), as well as to increase the patient’s awareness of when they are engaging in worry.

Mindfulness. Mindfulness interventions are based on focusing attention on something specific while withholding judgments about whether it’s good or bad. The present digital therapeutic provides both formal mindfulness meditation exercises (for example, mindful breathing, mindful eating, mindfulness to emotion) and more pragmatic or informal mindfulness exercises (for example, mindfulness to daily activities such as focusing on the feeling of wind on your face while walking to work or school, or mindfulness to daily chores like laundry or dishwashing).

Acceptance. Acceptance-based interventions involve shifting behavior to be effective and pragmatic by acknowledging the truth about one’s current situation and working within those constraints. The goal is to reduce unnecessary suffering due to engaging in ineffective behaviors. In this digital therapeutic, acceptance-based activities may include acceptance of difficult emotions such as anxiety, acceptance of uncertainty regarding the future, or acceptance of the possibility of future problems or negative outcomes.

Problem solving. These interventions provide basic skills in problem solving which patients may have failed to develop because they were either too anxious and avoidant to learn them or because they suffer from a neurocognitive problem that makes learning these kinds of skills difficult. Either of these circumstances may be directly or indirectly related to their migraine condition. The present digital therapeutic provides important skills for solving problems as an alternative to worrying about them. The patient may work to solve current problems by identifying and defining the specific problem to be solved, thinking through several alternative options available to them in a given situation, and choosing one that is good enough, even if it does not guarantee a successful outcome. For example, a patient who is struggling at work will learn to define the specific issue, e.g., workload is too heavy), generate several alternative solutions, e.g., communicate with supervisor about ways to manage or decrease workload; hire a new employee; quit job), and select and implement the most promising solution, e.g., communicate with supervisor).

Social skills training. These interventions may involve explicitly helping the patient practice interactions with others. These techniques are particularly useful for patients with high anxiety or neurodevelopmental problems.

Relaxation. The intervention may involve specific methods for reducing physiological arousal such as guided imagery or physical activities to reduce muscle tension. The digital therapeutic may include written instruction and audio recordings to help the patient develop skills in progressive muscle relaxation where they are instructed to tighten and release all major muscle groups in a specific order.

Goal setting. Goal setting interventions help patients to define concrete, specific, and achievable behavioral goals that are consistent with their values. For example, these interventions may help a patient to identify physical fitness as an important value and to set a corresponding goal of walking outdoors for 30 minutes each day.

Termination. These interventions focus on reviewing material learned to consolidate gains and prevent relapse. Making specific plans to cope with increased worry in the future is one example of this kind of intervention that is included in migraine digital therapeutic.

The present migraine related digital therapeutic delivers therapy in a sequence of modules of neurobehavioral interventions, patient education, and skill-building. It may be implemented in the form of a mixture of text, videos, quizzes, and interactions with a conversational artificial intelligence (Al) chatbot. The therapy is delivered via a software application intended to be used on a patient’s mobile device or any other computerized device, the software application accesses additional software associated with the digital therapeutic through a web-based portal or similar network access point.

Besides the patient’s application interacting with additional software as part of a patient’s treatment program, clinicians have access to a clinician dashboard that shows, among other things, how a particular patient is utilizing and engaging with the application, i.e., the digital therapeutic. Further, this dashboard provides access to relevant patient information for each patient regarding which a given clinician has authorization, such that the clinician may switch between patients as necessary. A fuller device description is provided herein.

The present digital therapeutic is a personalized treatment for patients suffering from migraine with related mental health issues such as MDD and GAD. The mental health component may be a directly or indirectly related to the migraine diagnosis or treatment or even completely unrelated thereto. It is based on empirically supported interventions from CBT among other neurobehavioral interventions. CBT is the term used for a group of psychological treatments supported by several decades of scientific evidence. Such therapies are sometimes short-term treatments that focus on teaching specific skills to a patient and have been shown to be effective in randomized clinical trials for MDD and/or GAD. Many such randomized trials utilized face-to-face delivery of treatment by a trained clinician. A feature of the present digital therapeutic is that such face-to-face interventions have been adapted to work in a digital format, i.e., utilizing software and networked connections between the patient and at least portions of the digital therapeutic software.

Further, the CBT-related aspects of the present digital therapeutic has been enriched with content from other neurobehavioral and related interventions including acceptance and commitment therapy (ACT) and positive psychology. ACT is a form of psychotherapy that has been extensively researched. It emphasizes interventions that use acceptance and mindfulness strategies while emphasizing the commitment to values and behavior-change strategies to increase psychological flexibility. positive psychology is the scientific study of the positive aspects of the human experience that make life worth living. Content from these related disciplines is reflected in some of the specific interventions delivered through the present digital therapy and are discussed further with regard to therapeutic modalities, where additional details are provided.

The migraine digital therapeutic can be personalized to the patient in ways that do not compromise the fidelity of the treatment and, in fact, are designed to markedly increase the efficacy thereof. Personalized mechanisms are discussed in details in sections below concerning the device overview and device personalization for the migraine digital therapeutic.

GAD has historically been more challenging to treat than other anxiety disorders, with a high number of patients continuing to report clinically significant symptoms after treatment (Borkovec & Ruscio, 2001). CBT for GAD typically emphasizes self-monitoring of worry (a cognitive process) and anxiety (an emotion) to increase early awareness of anxiety cues and behaviors, followed by skills to manage worry and anxiety spirals. These skills may include: changing thoughts and beliefs, relaxation training, scheduled “worry time,” planning pleasant activities, and controlled exposure to thoughts and situations that are being avoided. The migraine digital therapeutic described herein has incorporated each of these skills into its digital interventions. The therapeutic may also include acceptance-based approaches to increase mindful awareness and engagement in valued actions as well as skills for increasing tolerance of uncertainty. The digital therapeutic product represents an integrative approach because it appeals to the largest population without sacrificing safety or efficacy.

A substantial portion of the U.S. population are affected by MDD and/or GAD. Further, it is unsurprising that an even higher proportion of persons suffering from frequent migraines are also affected by MDD, GAD and/or related mental health issues. There is a clear need to provide therapeutic intervention for MDD and/or GAD on a large scale, and digital solutions can meet this requirement. 81% of Americans own a smartphone, and nearly 75% own a desktop or laptop computer (Pew Research Center, 2019). Psychological interventions that are delivered using these devices can help to increase access to care, which is a significant issue in the US with many depression and anxiety patients receiving suboptimal care or no care at all and can also help overcome barriers like the perceived stigma associated with mental illness. Digital interventions can help make effective treatments available more widely. There has been a substantial increase in research on such interventions, and the available evidence shows that these kinds of interventions can help specific disorders, even in patients with co-occurring medical conditions.

In addition to high prevalence in the general population, depression and anxiety disorders (like other mental health problems) are even more prevalent in people suffering from chronic medical conditions such as migraine. A global study of 42 countries concluded that several highly prevalent chronic physical conditions are significantly associated with depression and/or anxiety and having just one condition increased the odds of depression and/or anxiety symptoms by almost twofold. A person suffering chronic migraine, i.e., 15 or more headache days per month, has between 30 and 50% chance of depression. The rates of anxiety in chronic migraine suffers is even greater, estimated above 50%. Much of the anxiety felt is about when their next migraine attack will occur and how it will affect their life.

The present migraine digital therapeutic treats MDD and/or GAD with CBT interventions that modify or reverse maladaptive patterns of cognition and behavior. The specific targets for MDD and/or GAD include the following:
faulty estimates about the likelihood of negative events,
distorted views about one’s ability to cope with difficult events,
poor problem-solving skills,
positive beliefs about worry, e.g., as a way of preventing, problem-solving, or coping with negative events),
intolerance of uncertainty, and
cognitive, behavioral, and experiential avoidance.

At present, the digital therapeutic product described herein includes 112 total interventions aimed at modifying these processes. Additional details about the specific therapeutic modalities that are represented and included in the migraine digital therapeutic are provided below.

The present migraine digital therapeutic is a multi-week therapy that, for example, may be implemented as an 8-week digital therapeutic used to treat migraine, symptoms of migraine or side effects resulting from pharmaceutical treatment of migraine. Principles of CBT is an important component of the digital therapeutic, as are principals of ACT and positive psychology.

CBT is typically delivered by a clinician in a one-on-one format, although group formats are also sometimes used. Standard exposure of a patient to CBT usually occurs in weekly sessions over 8-14 weeks. CBT can be conceptualized as a skills-based treatment that delivers proven behavioral and cognitive treatment strategies. A ‘skills-based treatment’ may be contrasted, for example, with an insight-oriented treatment.

Among many other advantages of a digital therapeutic is the flexibility of ‘dispensing’ and ‘dosing’ treatment. That is, the digital and networked nature of the treatment means that there is no need to schedule treatment(s) based on availability of a healthcare professional (HCP) or other factors, nor does there need to be any considerations of travelling to the HCP’s office. It has been found that a dosing of two digital therapeutic interventions per day is easily achievable by the average patient. Other than in an in-patient setting, such a therapy frequency is completely unattainable. This being the case, a battery of CBT substantially shorter than the typical 8-14 weeks may be achieved. The present migraine digital therapeutic may have a duration of between about four weeks and fourteen weeks, including durations of about four weeks, six weeks, eight weeks, ten weeks, twelve weeks and fourteen weeks. However, the product’s design allows flexibility to accommodate the needs of the patient, e.g., for missed interventions. Patients may be provided access to the migraine digital therapeutic for more weeks than typically required to complete to provide additional accommodation.

The availability of the digital therapeutic may be set to end automatically based on the start date, i.e., the date the patient creates an account and begin the treatment. A variable number of interventions may be unlocked each day. For many patients, two interventions per day seem to encourage ongoing engagement with the therapy. One, three or four interventions are, however, both feasible and even advisable for some patient groups. Obviously, if the time commitment and/or complexity of interventions were adjusted significantly upward or downward, this would have an impact on the appropriate dosage per day for the average patient. In the event that a patient does not complete the total number of interventions set for a given day, they may be required to do so before new interventions are unlocked. The ‘flow’ and order of interventions for a digital therapeutic has the potential to significantly impact the efficacy of the therapy.

The number of interventions in a particular digital therapeutic, as well as the order and flow of these interventions, will be an important factor in the efficacy of a digital therapeutic. For example, completion of two interventions per day for eight weeks has been measured as delivering efficacy for indications such as MDD and GAD. The entire treatment course under these circumstances would involve completion of one hundred and twelve interventions. Again, the potential exists for extending the duration of treatment so a patient can complete the full course of treatment. In addition, such flexibility as permitting patients to make up one intervention per day, for a total of three interventions in a day, may be used to keep a patient to schedule.

The migraine digital therapeutic delivers neurobehavioral interventions in a sequence of four modules: (1) learn about worry (2) reduce suffering (3) increase joy and meaning, and (4) maintain progress. See, e.g., FIG. 1. These interventions are delivered in four key media formats: 1) conversation guided by an AI chatbot, 2) audio recordings, 3) video recordings, and 4) quizzes, each module is described in detail hereinbelow.

The present digital therapeutic may request patients complete a daily worry diary as part of the therapeutic intervention. This diary may be completed through interactions with the AI chatbot. An advantage of delivering the worry diary via the AI chatbot is that it allows patients to ask questions and receive guidance if needed. This patient may monitor and record worry episodes and topics and situational and internal triggers for worry and associated emotions. It is intended to help give the patient a clear picture of their worry and increase the patient’s awareness of when they are engaging in worry - which is particularly important because worry is a covert event. As patients become more familiar with their worry patterns, they will also learn to categorize each thought as 1) worries about current problems that may respond to in-the-moment problem-solving or 2) worries about potential problems that may never actually come to pass and will be managed with acceptance and mindfulness. Insights from self-monitoring will be used later in the treatment.

Psychoeducation is also provided throughout all modules of the treatment and may include information about treatment rationale, common pitfalls, and scientific models about how thoughts, behaviors, and emotions influence migraine, migraine symptoms, migraine treatment side effects, GAD and MDD.

The presently described migraine digital therapeutic may be personalized to address key interest areas, increase engagement and accomplish other important efficacy goals, in several ways:
1. Information the patient provides through onboarding questionnaires and responses to the AI chatbot is saved, probably ‘remembered’ may be a better term, to be used later in the treatment. This information may be referred to as “life graph variables” that can be accessed to give the AI chatbot dialogues a more personalized feel. For example: if a patient tells the AI chatbot that he has a dog, she will ask the dog’s name and store it under the life graph variable “dog_name.” The chatbot may then use that name in any future task. If the patient completes an intervention two weeks later and suggesting that he go for a walk is appropriate, the AI chatbot may reference that name in the suggestion in the following way, “Perhaps you could take a walk with your dog Lolly.” If the life graph variable dog_name is empty, Anna will say, “Perhaps you could take a walk.”
2. The patient may be provided with the option of completing the MDD and/or GAD treatment with an emphasis on family concerns, career stress, or physical wellness, as further discussed below. If they choose one of these options, the treatment content may be modified with examples relevant to that area of interest. It is not necessary to change the number of interventions or the therapeutic modality of the interventions to achieve this. Rather, interventions may simply be adapted to better reflect the patient’s interest. This approach utilizes Happify’s Indication Treatment Sequence described below. Briefly, this sequence specifies each intervention’s therapeutic modality and the order in which those interventions are to appear. For example, day 19 of the treatment has one intervention that delivers a brief mindfulness exercise and one intervention that delivers psychoeducation regardless of whether the patient is completing the version that emphasizes family concerns, career stress, or physical wellness. On day 19, the mindfulness exercise content is adapted slightly, and examples within the psychoeducation intervention are modified based on which experience is offered, but the core therapeutic elements remain the same.
3. The migraine digital therapeutic can adapt to treat MDD and/or GAD in specific populations, notably in people suffering from chronic medical conditions, such as migraine, asthma or psoriasis. For these cases, the digital therapeutic may enrich psychoeducation with information about the medical condition and MDD and/or GAD and ensures that all examples and suggested activities are appropriate for people suffering from that condition. For example to adapt for a patient with migraine, the digital therapeutic may replace recommendations to go for a run with recommendations meditate instead. Changes may follow an indication treatment sequence, discussed further below, and be personalized to ensure the suggestions are appropriate for the intended subpopulation.

The migraine digital therapeutic presented herein is a software application intended to offer at-home treatment for migraine, GAD and/or MDD in an engaging, user-convenient format as a prescription or over the counter digital therapy. This design is expected to result in a safe, effective, and convenient treatment option that supports patients’ compliance and offers a favorable risk-benefit profile.

The Instructions for Use (IFU) for the present therapeutic may note that it should be used under the supervision of a licensed Health Care Provider (HCP) and it is not meant to be a substitution for any treatment medication. The IFU will also include product specific warnings and contraindications.

The migraine digital therapeutic may include a variable number of neurobehavioral modules, with the specific number of modules determined by a number of factors. Similarly, the number of interventions per module is also an important factor in designing the digital therapeutic. Important factors in making these determinations are efficacy of the treatment and retaining engagement of the patient for the entire course of treatment. Experiments involving actual patients may be conducted with varying numbers of modules and interventions may be utilized to achieve efficacy and patient retention. Experimental data has been collected to general uses of digital therapeutics as well as toward specific indications treated by such therapeutics, e.g., migraine, MDD and GAD. Further, since many interventions and even whole modules are potentially useful across indications and mental health disorders, much knowledge has been gathered by developers of the present digital therapeutic that is useful in determining how to maximize efficacy and patient retention as well as in designing experiments of the type discussed. The present migraine digital therapeutic may utilize four neurobehavioral intervention modules and provides performance feedback to patients and clinicians.

Looking to a digital therapeutic focused on treating GAD, the first module is organized to achieve three treatment goals:
1. To educate patients on key concepts related to worry and anxiety (psychoeducation)
2. To increase patients’ awareness of relationships among thoughts, actions, and emotions related to worry (daily monitoring)
3. To improve patients’ skills for identifying and targeting current problems, defining goals, and brainstorming and implementing possible solutions (problem-solving)

The first module focuses on introducing patients to the treatment program and setting the right framework for success. It begins with education about GAD symptoms followed by daily monitoring of thoughts, actions, and emotions related to GAD. The educational content is focused on the nature of anxiety and worry and common misconceptions about the value of worry. For example, many people with MDD and/or GAD believe that worrying protects them or that worry is required if you love someone. Self-monitoring involves paying attention to worry episodes and topics along with situational and internal triggers and associated emotions. Self-monitoring is intended to help give the patient a clearer picture of their worry and increase the patient’s mindfulness when they are engaging in worry, which is particularly important because worry is a covert event. It helps patients see how the diagnosis affects them as individuals. Next, problem-solving is introduced to ensure the patient has a viable alternative to worry.

As patients become more familiar with their worry patterns, they will also learn to put worries into two categories: 1) worries about current problems that may respond to in-the-moment problem-solving and 2) worries about potential problems that may never actually come to pass and will be managed with acceptance and mindfulness. Problem-solving skills are introduced to address worries in category 1. Many individuals with GAD see problems as a threat of failure, avoid facing current problems, or lack practical problem-solving skills. These skills include correctly identifying current problems, defining goals, and brainstorming and implementing possible solutions.

Interventions in Module 2 for GAD may be organized to achieve three treatment goals:
1. To educate the patient on critical concepts related to mindfulness and acceptance
2. To increase nonjudgmental awareness and acceptance of experiences in the present moment
3. To increase psychological flexibility and willingness to tolerate uncomfortable experiences and emotions

Mindfulness and acceptance-based techniques help patients to replace future-focused worry and anxiety with nonjudgmental awareness and acceptance of experiences in the present moment. It includes increasing psychological flexibility and willingness to tolerate uncomfortable experiences and emotions, including the anxiety and uncertainty inherent in life. For example, nobody knows for sure if they will have a job in two weeks and no worrying can change that. Therefore, patients are encouraged to observe and sit with the uncomfortable emotions and sensations associated with that reality. This module will include psychoeducation about mindfulness and acceptance, along with formal and informal mindfulness exercises. Mindfulness and acceptance-based techniques are useful for many purposes, including MDD and/or GAD.

Interventions in Module 3 may be organized to achieve three treatment goals:
1. . To help patient to identify their values and the ways in which worry and anxiety interfere with valued activities
2. To help patient set goals related to their values
3. To increase patient’s mindful engagement in valued activities despite the anxiety

Module 3 emphasizes increasing engagement in activities motivated by the patient’s values, rather than by anxiety or worry. Worry and anxiety often interfere with patients’ engagement in valued activities. Even if they are going through the motions of participating in valued activities, the worry and anxiety may distract mindful focus on these activities and reduce meaning and satisfaction. This module’s primary goal is to help patients move from a place where their activities are dictated by avoidance of worry, anxiety, or feared negative outcomes to a place where they mindfully and fully engage with valued activities despite anxiety. This module includes exercises to help patients to identify their values and make specific plans to engage mindfully in values-driven activities and goals, despite anxiety.

Interventions in Module 4 are organized to achieve three treatment goals:
1. To consolidate gains and maintain progress into the future
2. To review key concepts
3. To make a plan for how to cope with future worries and anxiety

The final module is focused on consolidating what the patient has learned and maintaining improvement in symptoms. Key interventions emphasize positive psychology to ensure a focus on continued growth and flourishing and planning & termination interventions. Key themes from psychoeducation are reviewed, and skills are practiced. Patients are guided through creating a list of helpful knowledge and skills that they can review in the future if anxiety increases. This section provides a helpful framework for thinking about relapse as a challenge that can now be met with greater success than patients would have had before treatment with migraine digital therapeutic.

A migraine focused digital therapeutic may incorporate performance feedback to both the prescribing clinicians and to the patients using the product.

A validated self-report measure of GAD symptoms, The Generalized Anxiety Disorder Scale-7 (GAD-7), is administered by the product as part of the treatment. The GAD-7 is one of the most frequently used diagnostic self-report scales for screen, diagnosis and severity assessment of anxiety disorder and it was developed by Drs. Robert L. Spitzer, Janet B.W. Williams, Kurt Kroenke and colleagues. Patients using the presently described migraine digital therapeutic are requested to complete GAD-7 scale during their treatment. For example, a GAD-7 assessment may be done prior to treatment as a baseline and then every week or every two weeks throughout treatment.

GAD-7 scores of 5, 10, and 15 are reported to the patient as indicating mild, moderate and severe anxiety respectively. Raw scores may be presented to the patient with additional text to explain what the score means. The migraine digital therapeutic may use the standard cutoff scores recommended in the scoring manual. These scores may be cumulatively graphed and presented to patients immediately following each completion of the measure, allowing them to easily track and understand their progress over time. Data from the GAD-7 may also sent to the prescribing physician. Feedback to the prescribing clinician may be delivered through a secure clinician portal. Clinicians will be able to log into the portal at any time to see compliance statistics and the performance metrics described above. Patients using the product will likely be informed that this information is being shared with their clinician.

The migraine digital therapeutic may include a conversational AI chatbot feature designed to mimic human interaction. The chatbot may be referred to utilizing a human name, e.g., “Anna” or the like, so as to give it a more personalized touch, but it is clearly stated to the patient that this is a computer, not a real person. Anna may guide the patient’s engagement with each intervention via a conversational dialog that responds to the patient’s text. In many cases, this may involve greeting the patient and collecting information. Anna employs a mix of instruction and feedback that includes open-ended questions, multiple-choice options, and clarifying examples to guide the patient.

In addition to the clinical interventions delivered daily, polls and games are designed to make the experience more enjoyable for the patients. These engagement features are not necessarily considered part of the dosing or the therapy. The patients are not required to interact with these features but may earn a “gold medal” if they complete all required interventions in a module within the prescribed time frame. Those who require extra time to complete a module may receive a silver medal. Also, the migraine digital therapeutic may be designed with a community feature to share activities the patients have completed, newsletters, and infographics containing relevant information about mental health.

The current migraine digital therapeutic was designed to be personalized without compromising its clinical efficacy. The treatment may be set to automatically adapt based on one of a plurality of areas of interest chosen by the patient. It also includes minor personalization elements, like remembering the names of important people in the patient’s life, through the chatbot Anna. The migraine digital therapeutic intervention can be adapted for work with a specific subpopulation of people suffering from migraine, such as those who have GAD, MDD or particular side effects from a migraine medication. The method for accomplishing these kinds of personalizations without compromising the treatment’s clinical efficacy is described in detail below. The sections may provide detailed explanations of personalization and justification for its need in migraine digital therapeutic.

The migraine digital therapeutic may deliver brief (10-20 minute) daily interventions based on CBT and enriched with techniques from positive psychology and ACT. Each of these daily interventions can be categorized into the specific modality it represents. The developers have identified 12 therapeutic modalities into which psychological interventions can be classified. Examples of therapeutic modalities include mindfulness and behavioral interventions, with three examples, each of the specific interventions that would fall under each modality. The migraine digital therapeutic interventions may fall within the following therapeutic modalities: psychoeducation, monitoring, mindfulness, relaxation, behavioral interventions, acceptance interventions, problem-solving, positive interventions, and termination. This system of labeling each intervention with its appropriate therapeutic modality, among other functions, helps link each intervention to the scientific literature supporting its efficacy.

Activities, Tracks, Interventions, Modules and Interventions

| Activity ID | Skill | Activity Type | Activity Name | Level (1-5) |
|---|---|---|---|---|
| A-01 | Aspire | Essay | My victorious self | 1 |
| A-02 | Aspire | Essay | My core values | 3 |
| A-03 | Aspire | Essay | What’s My Why? | 1 |
| A-04 | Aspire | Plan-Do | I think I can | 2 |
| A-05 | Aspire | Essay | Find meaning in the mundane | 2 |
| A-06 | Aspire | Essay | Setbacks and steps forward | 4 |
| A-07 | Aspire | Essay | Pursue meaning | 3 |
| A-08 | Aspire | Plan-Do | Create meaning | 5 |
| A-09 | Aspire | Plan-Do | Spend on what matters | 4 |
| A-10 | Aspire | Essay | I have a goal | 5 |
| A-11 | Aspire | Essay | I’m looking forward to... | 1 |
| A-12 | Aspire | Plan-Do | Developing and Using My Strengths | 2 |
| E-01 | Empathize | Essay | Give myself a break | 1 |
| E-02 | Empathize | Essay | Walking in their shoes | 2 |
| E-03 | Empathize | Essay | Whats my positive impact? | 1 |
| E-04 | Empathize | Essay | Weird..why’d they do it? | 2 |
| E-05 | Empathize | Plan-Do | Get to know someone | 3 |
| E-06 | Empathize | Essay | Empathize with a different viewpoint | 3 |
| E-07 | Empathize | Essay | Not cool - why’d they do it? | 3 |
| E-08 | Empathize | Essay | Empathize when you disagree | 4 |
| E-09 | Empathize | Essay | That hurt! What made them do it? | 5 |
| E-10 | Empathize | Plan-Do | Help someone | 4 |
| E-11 | Empathize | Essay | There’s No One Like Me | 4 |
| E-12 | Empathize | Essay | Empathize when you’re resentful | 5 |
| E-13 | Empathize | Do | Create a micro-moment of connection | 2 |
| G-01 | Give | Plan-Do | Give a small gift | 1 |
| G-02 | Give | Do | One day, 5 nice things | 4 |
| G-03 | Give | Plan-Do | Make someone smile | 1 |
| G-05 | Give | Plan-Do | Spend time on a valuable activity | 2 |
| G-06 | Give | Plan-Do | Spend $$ on a shared experience | 3 |
| G-07 | Give | Plan-Do | Celebrate someone’s good news | 4 |
| G-08 | Give | Essay | Forgive an annoyance | 4 |
| G-09 | Give | Plan-Do | Spend time with someone | 3 |
| G-10 | Give | Essay | Forgive an offense | 5 |
| G-11 | Give | Plan-Do | Volunteer | 5 |
| G-12 | Give | Plan-Do | Donate $$ for a cause | 5 |
| R-01 | Revive | Plan-Do | Sleep: Brighten Your Wake-Up Routine | 1 |
| R-02 | Revive | Audio | Sleep: Relax with Autogenic Training | 2 |
| R-03 | Revive | Plan-Do | Sleep: Wind Down | 3 |
| R-04 | Revive | Plan-Do | Sleep: Optimize Your Sleep Schedule | 4 |
| R-05 | Revive | Plan-Do | Body/Fitness: Take a Stand | 1 |
| R-06 | Revive | Do | Body/Fitness: Breathe Deeply | 1 |
| R-07 | Revive | Plan-Do | Body/Fitness: Make Time to Move | 2 |
| R-08 | Revive | Plan-Do | Body/Fitness: Feel the Stretch | 2 |
| R-09 | Revive | Plan-Do | Body/Fitness: Build Up Your Core | 3 |
| R-10 | Revive | Plan-Do | Body/Fitness: Commit to a Class | 4 |
| R-11 | Revive | Plan-Do | Body/Fitness: Rev Up Your Resistance | 4 |
| R-12 | Revive | Plan-Do | Body/Fitness: Commit to a Fitness Routine | 5 |
| R-13 | Revive | Do | Nutrition: Boost Your Water Intake | 1 |
| R-14 | Revive | Plan-Do | Nutrition: Start Your Day with Protein | 2 |
| R-15 | Revive | Plan-Do | Nutrition: Eat More of the Good Stuff | 2 |
| R-16 | Revive | Plan-Do | Nutrition: Start a Healthy Snack Habit | 3 |
| R-17 | Revive | Plan-Do | Nutrition: Meal-Prep Makeover | 4 |
| S-01 | Savor | Plan-Do | Savor the small stuff | 1 |
| S-02 | Savor | Do | Body scan meditation | 1 |
| S-03 | Savor | Plan-Do | Savor together | 2 |
| S-04 | Savor | Essay | Savor a memory | 2 |
| S-05 | Savor | Do | Avoid overthinking, | 3 |
| S-06 | Savor | Essay/Do | Basic meditation | 3 |
| S-07 | Savor | Plan-Do | Moving meditation | 3 |
| S-08 | Savor | Plan-Do | All-day savoring | 4 |
| S-09 | Savor | Essay | Reframe negative thoughts | 4 |
| S-10 | Savor | Plan-Do | Walking meditation | 4 |
| S-11 | Savor | Plan-Do | Organize a savoring event | 5 |
| S-12 | Savor | Essay/Do | Living in the moment | 5 |
| S-13 | Savor | Do | Loving-Kindness Meditation | 3 |
| T-01 | Thank | Essay | Weekly Gratitude check-in | 2 |
| T-02 | Thank | Plan-Do | A week’s worth of thanks (about a person) | 2 |
| T-03 | Thank | Plan-Do | Deliver a week’s worth of thanks | 3 |
| T-04 | Thank | Essay | Thx Thx Thx | 1 |
| T-05 | Thank | Essay | What am I proud of? | 3 |
| T-06 | Thank | Do | Thank you note | 4 |
| T-07 | Thank | Do/Plan-Do | Deliver a thank you note | 5 |
| T-08 | Thank | Plan-Do | I’m thankful, let’s talk! | 4 |
| T-09 | Thank | Essay | Today’s grateful moment | 1 |

Exemplary text and an explanation as to psychological theory underlying the activities is presented below in for each activity:

| | | |
|---|---|---|
| Activity ID | “You Decide How” Text | Why It Works |
| A-01 | Imagine everything you would have hoped to happen in the next few years actually DID happen. Meet your “Victorious Self.” Think about your future self in two, five, 10 years—whatever works for you. Imagine your life from all angles: your friends, where you live, what you do, how you feel about yourself, and how you’ve changed. Write specific details about what you’ve imagined and how it feels, touching on your personal life, relationships, and accomplishments at home and at work. Be both realistic— and optimistic! | Research shows that imagining a better future can actually make us happier—today. It clarifies our priorities and boosts our optimism. In a 2010 study, subjects who imagined a “best possible self” for one minute and wrote down their thoughts generated a significant increase in happiness. The researchers also concluded that the exercise increased the likelihood for someone to expect a positive future. If we feel optimistic about our goals, we’re more likely to invest the effort in reaching them. When we imagine and write about succeeding at the goals we’ve set for ourselves, it leads to increases in well-being and even physical health. In a study asking participants to write descriptions of the best possible version of their future selves for 20 minutes, four days in a row, researchers found that when compared with other writing prompts, this exercise was associated with positive mood and enhanced well-being three weeks later, and a decreased rate of illness five months later. In a variant on this exercise by Sheldon and Lyubomirsky, people who visualized and wrote about their best possible selves over four weeks also showed higher levels of motivation and interest than those who were simply asked to write about life details. Visualizing a future best self can be a powerful catalyst for change. It can raise our expectations for the future, allowing us to break out of shackling beliefs and set us in motion to deliver on great things for our future selves. |
| A-02 | Defining your values will help you define your life. If you take the time to really think about what’s important to you, you’ll feel more committed to living them, and be able to face life’s challenges with increased confidence. Take a few minutes to write down your values. At your best, what kind of person are you? What positive traits would you like to work towards developing? To get you thinking, here is a short list of common virtues, but | Defining your values will help you define your life. If you take the time to really think about what’s important to you, you’ll be able to face life’s challenges with increased confidence. And if you take the time to write down your values, you’ll feel more committed to living them. Studies found that when we engage in activities that are meaningful to us, we’re more inspired, satisfied, motivated and happy. We’re more self-confident, we have a greater sense of purpose, and we feel more connected. And, we’re better able to handle life’s challenges. Once we know our values, we have a valuable checklist for goal-setting. When our goals are in sync with our values, |
| | feel free to come up with your own: confidence, creativity, education, faith, family, friends, fulfillment, fun, happiness, health, integrity, intimacy, kindness, peace of mind, status, success, wealth, wisdom... to name a few. | we’re likely to pursue them with more purpose, more satisfaction and more success. Research that meaning in life correlates positively to mood stability and sociable behavior. Meaning at work seems to inspire motivation and engagement. Individuals who feel their work relates to a higher purpose, for example, are more satisfied at work and also devote more effort to their job. |
| A-03 | Think about the activities in your life that are the most meaningful to you— what motivates you, excites you, moves you? If you don’t know, and that in itself is one of your concerns, take a look at the categories below and pick two or three that fit: Parenting, Family, Friendships/Socializing, Education/Personal Growth, Career, Recreation, Spirituality/Religion, Physical Health, Helping Others. Now write a few thoughts about how these topics are meaningful to you and why. | Studies have found that when we engage in activities that are meaningful to us, we’re more inspired, satisfied, motivated and happy. We’re more self-confident, we have a greater sense of purpose, and we feel more connected. We’re better able to handle life’s challenges. Research shows that meaning in life correlates positively to mood stability and sociable behavior. Meaning at work seems to inspire motivation and engagement. Individuals who feel their work relates to a higher purpose, for example, are more satisfied and devote more effort to their job. |
| A-04 | Think of something you’d love to achieve by the end of the week— something that matters to YOU (not something your partner or boss or friend wants you to do)—or a task you’ve been avoiding. It could be reconnecting with an old friend or cleaning out your garage. Now jump ahead and imagine that you’ve just completed your goal. How is it making a difference in your life? What’s the feeling of accomplishment like? Write down your short-term goal and your reflections about completing it. Then jot down a few ways—“baby steps”—you might actually start to get there. Finally, go for it! And come back to report how you pulled it off and how it makes you feel. | Professors Edwin Locke and Gary Latham have studied goal-setting methods for decades. In numerous studies, they’ve discovered the most successful goals are those that are both “challenging and specific” - in other words, they should be ambitious and they should be measurable. Another study led by Bruce Headey shows that our “choice” of goals also plays a role in our subjective well-being. Goals that are non-competitive (commitment to relationships, helping others, community) promote our life satisfaction whereas competitive goals (job advancement, making more money) are actually detrimental to our satisfaction with life. Focusing on the process, rather than the outcome, can help you achieve your goals. Research by Pham and Taylor found that students who visualized themselves studying for a test performed better than students who visualized themselves doing well on the test. Those who visualized the process (i.e. studying) were more likely to study, which ultimately had the largest effect on their test performance. If you own your own goals—and then take steps towards them—you’re on your way to increasing your overall well-being. Studies show that when people pursue goals intrinsic to them, they’re more motivated, more likely to succeed, and far happier than people who don’t have or don’t pursue strong dreams or aspirations. |
| A-05 | Routines are boring. Daily chores are | Research shows that doing something tedious in a different |
| | boring. Life can be boring. The key is to power through things we don’t want to do, because procrastination is deadly—and an avoidable stressor. So take your least favorite activities and imagine you’re living in some pretend society that places enormous value on the people who perform that activity. How do they benefit from you engaging in this activity? Now carry out that task with purpose and try to come up with reasons it’s important. Think of the guys who repetitively slice, dice, and roll sushi pieces for hours on end. That can’t be that much fun, right? And yet most all of them have a razor focus on their work, perhaps because they’ve been trained to believe they are creating meaningful, artistic products. | way can help people stay focused and more motivated. When we consider the purpose of an activity, our attention shifts from the little mundane details to more abstract concepts and principles. Our beliefs about the activities we partake in can have a significant effect on their outcomes. Researchers Ellen Langer and Alia Crum split 84 hotel workers into two groups, one a control and the other given information about how the work they do (cleaning rooms) serves as good physical fitness along with clear examples. In just four weeks, the control group had no measurable physical changes, but the test group showed a number of changes including decreases in weight, blood pressure, and body fat. Simply thinking about an activity as being good for you or good for others can help reinforce its positive effects. |
| A-06 | Think of a setback or negative experience that occurred at least one year ago. How has that event shaped who you are today? Can you think of any benefits that came of it? Spend ten minutes writing about its impact on your life. | The most successful people are those who can actively learn from their mistakes, handle rejection and move forward. In other words, failure IS an option. What matters is how you respond to it. Over the years, researchers have found that asking people to participate in expressive writing about stressful experiences has beneficial psychological effects, reducing stress and producing long-term improvements in mood. When asked to think about the good things that came out of a bad experience, the benefits were even greater. People who engage in “benefit-finding” generally report less distress, fewer disruptive thoughts, less negativity, and more meaningfulness in their lives. Writing about extremely emotional and personal topics also has a positive health effect: One study found that in participants with elevated blood pressure, it lowered blood pressure for several months following the exercise. |
| A-07 | If you brainstormed about your core values in a previous activity, you may have come up with things like integrity, healthy living, or kindness. If you haven’t yet, take a few moments to write down your values. (If you take the time to really think about what’s important to you, you’ll feel more committed to living them, and be able to face life’s challenges with increased confidence.) | If you take the time to really think about what’s important to you, you’ll be able to face life’s challenges with increased confidence. Research shows that meaning in life correlates positively to mood stability and sociable behavior, and meaning at work seems to inspire motivation and engagement. Going after goals that are important to us also has a slew of psychological and physical benefits. Goals that include commitment to something outside ourselves, such as our family, friends or community, promote life satisfaction. Zero-sum goals, such as a commitment to material gains or career success, actually |
| | Now take what you learned and brainstorm concrete ways you can make change your everyday life to better pursue the character ideals you identified as being most important. | can be detrimental to our satisfaction with life. Studies show that when we pursue goals that are meaningful or intrinsic to our values, we’re happier, more motivated, and more likely to succeed. |
| A-08 | Take an everyday activity you do already, and transform it into something meaningful. So if you’re always tinkering with new recipes, compile your best ones into a book that you can pass along to your grandchildren one day. If you run every morning, train a group of teens to run a half-marathon. | When we set meaningful goals, we begin to direct our energies in a way that aligns with what’s important to us. And when we do something that we believe is meaningful, we feel good and achieve a sense of purpose in our lives. Studies have consistently found that the more meaning people report in their lives, the happier they are—and this is true of people in all life stages. You don’t necessarily have to help others in order to make your activity meaningful, but when you do, it amplifies the effects of that activity and brings about greater well-being. Research shows that helping others distracts us from our own thoughts and concerns. Anxiety and stress involve a high degree of focus on self, and focusing on the needs of others helps us shift our thinking and boosts our self-esteem. |
| A-09 | Spend some money—it doesn’t have to be a lot!—on a leisure experience that will be meaningful and rewarding for you (concentrate on experiences, rather than material things). For example, if you’re passionate about music, take a music class or go to a concert. If you’re a history buff, you might visit a museum or historic site near your town. | Studies show that people who spend money on experiences are happier than those who spend money on material possessions. Studies show this to be true across most demographic categories: male or female, liberal or conservative, high or low income and religious preferences. Life experiences become part of who we are. They’re woven into our memories and they shape our identity. In one study, researchers found that spending money on positive experiences increased people’s happiness. And the study showed that people who spent money on a series of smaller events were happier than those who spent money on one big event. In other words, you’ll be happier if you spread out your positive experiences rather than spending money on “peak” experience. |
| A-10 | Set a very long-term goal—one that can be completed in the span of several years. Maybe you finally complete the memoir you’ve wanted to write for years. Or, you go back to school in order to make a major career change. Imagine the benefits of achieving that goal. What will happen? How will you feel? | Professors Edwin Locke and Gary Latham have studied goal-setting methods for decades. In numerous studies, they’ve discovered the most successful performance goals (ones that can be measured, like getting a $10,000 salary raise or completing a half-marathon) are those that are both challenging and specific, meaning they can be clearly defined. If we set the right goals—and then take steps towards them—we get on the path to increasing our overall well-being. Studies show that when people pursue goals intrinsic to them, they’re more motivated, more likely to succeed, and far happier than people who don’t have or don’t pursue strong dreams or aspirations. Intrinsic goals come from your |
| | | genuine values, beliefs, and interests, rather than the promise of fame or money, or the wishes of others. Focusing on the process, rather than the outcome, can help you achieve your goals. Research by Pham and Taylor found that students who visualized themselves studying for a test performed better than students who visualized themselves doing well on the test. Those who visualized the process (i.e. studying) were more likely to study, which ultimately had the largest effect on their test performance. When we set meaningful goals, we begin to direct our energies in a way that aligns with what’s important to us. When we do something that we believe is meaningful, we achieve a sense of our own worth and place in life. And studies have consistently found that the more meaning people report in their lives, the happier they are—and this is true of people in all life stages. |
| A-11 | What upcoming events are you anticipating with joy and optimism instead of anxiety? Think of a future event, big or small, that you’re really excited about. Imagine the details, including the sounds, smells, and tastes. Spend some time putting yourself right in the middle of it. | When we find ourselves thinking about our future, we’re usually worrying about something, increasing our levels of anxiety. Even when we have upcoming events we’re truly excited about, most of us don’t take advantage of the anticipatory experience. Research shows that anticipating upcoming events prolongs our excitement and boosts our optimism. Optimistic people are more likely to persevere when the going gets tough. They’re good copers. They don’t give up. When we have something to look forward to we feel more enthusiastic, motivated, and psyched. And we’re more likely to behave in ways that ensure our positive future event will happen. Studies show that the act of anticipating future events also lowers our levels of cortisol, the stress hormone, because when we believe good things are coming our way, we’re happier in the day-to-day. |
| A-12 | What makes each of us special is that we all have unique character strengths—and when we use those strengths in novel ways, we become happier. Once you’ve taken the VIA survey to determine your top five strengths, come up with a way to use one of your strengths in a new and different way this week. For example, if one of your top strengths is appreciation of beauty and excellence, go to a museum you’ve never been to before. If your strength is kindness, you could leave a big tip on a small check. If your strength is courage, look for an opportunity to stand up for someone else who needs support. You | Gaining insight into what your strengths are—and then using more of those strengths in your daily life—can have a major impact on your happiness. Christopher Peterson, Martin Seligman, and their colleagues classified 24 signature character strengths (now published as the Values in Action (VIA) Classification), including courage, wisdom, curiosity, fairness, and self-regulation, that serve as the essential building blocks of positive character. Research has shown that using your top character strengths in novel ways can have long-term effects on your well-being. In one study, people took a survey to identify their character strengths and received feedback about their top five strengths. Then, they were asked to use one of their top strengths in a new and different way, every day for a week. After one-month, three-month, and six-month follow-ups, people who continued to find new ways to exercise their strengths saw the biggest increases in happiness and the |
| | get the idea! | biggest decreases in depression. (Merely identifying signature strengths can lead to a quick mood boost, but it doesn’t have the long-term effects of actually using your strengths.) Using signature strengths at work has also been linked to increased happiness and job satisfaction, and strengths-based school interventions have shown to improve student’s social skills, engagement in school, and “learning” strengths (like greater curiosity and an increased love of learning). |
| E-01 | As you go through your day, pay attention to your inner voice - and when you catch your inner voice berating you for something or other -catch yourself. Come up with with a phrase or mantra to shift your thinking. “Live and Learn” or “This too shall pass” - or whatever works for you. Then, focus on what makes you great. What have you overcome or achieved? You can even create a narrative of your life thus far, highlighting the aspects that showcase your positive traits. | Self-compassion boosts our resilience so that when we stumble, we can get up and try again. When making a mistake isn’t a big deal, we’re more open to learn from our missteps and try new things, rather than hide in shame. Kristin Neff, Ph.D., an associate professor at the University of Texas at Austin, has been researching self-compassion for well over a decade. Neff believes today’s competitive culture leads us to believe being good enough isn’t good enough. Media images and slogans push us to compare ourselves with others, even though there will always, always be someone who is more successful, richer, skinnier, or more attractive than we are. Neff’s research shows that self-compassionate people feel more secure and strongly suggests that accepting our imperfections may be the first step toward better health. People who are easier on themselves are less likely to be depressed and anxious and are more prone to happiness and optimism. Self-compassion steps in precisely when we fall down, allowing us to get up and try again. |
| E-02 | Pick a person and spend some time imagining what it’d be like to be them. What is life like for them? What must they be feeling and why? Write down some thoughts about your experience. | Research clearly suggests that when we empathize with others and try to see the world through other’s eyes, we’re more likely to treat them with kindness. And when we appreciate someone else’s point of view, he/she will most likely become more sympathetic and understanding of ours. In studies, scientists have found that people are generally kinder and more helpful after taking others’ perspectives, as it increases compassionate emotions towards the other person. This exercise also leads us to view and treat others more like ourselves and attribute more positive traits to others. That alone is a pretty good reason to start exercising our compassion muscles. |
| E-03 | Think of someone you’ve positively impacted, whether it’s someone from your inner circle or a complete stranger. Now imagine what it’s like for him or her to be on the receiving end of your kindness. | Most of us spend time dwelling on our negative traits and often neglect to recognize our virtues. One study showed that reframing negative thoughts and boosting self-esteem through someone else’s perspective is a very effective way to increase resilience, resourcefulness, optimism and positive thinking. Research shows that self-compassion comes with mental |
| | What positive impact have you had on his or her life? Are there ways you could be even more helpful? | health benefits like less depression, more optimism, greater happiness, more life satisfaction. Scientists have also found that people who respond to life’s challenges with self-compassion are happier, healthier, more proactive, and more conscientious than those who are not. People who are kind to themselves seem to take better care of themselves AND others. Self-compassionate people are more resilient, feel more connected to others, and are less depressed and anxious than others. So when you find yourself feeling guilty for not doing enough, consider the positive impact you have on the people around you and have compassion for YOURSELF. |
| E-04 | You’re right, they’re wrong. Okay, fine, sure, whatever. But that attitude isn’t going to get you far when it comes to patching things up. Time to try a different approach. When someone close to you does or says something that you don’t understand, try looking at the situation a little differently. What led up to it? What context might better explain his/her behavior? We all do strange things once in awhile, especially when we’re tired, hungry, angry or lonely. | Research clearly suggests that when we empathize with others and try to see the world through other’s eyes, we’re more likely to treat them with kindness. And when we appreciate someone else’s point of view, he/she will most likely become more sympathetic and understanding of ours. Studies have found that people are generally kinder and more helpful after perspective-taking, as it increases compassionate emotions towards the other person. This exercise also leads us to view and treat others more like ourselves. Scientists found that when we actively try to understand another person’s perspective, we think of them more positively afterwards. |
| E-05 | Think of someone you cross paths with but rarely speak to—someone who is different than you. Plan to strike up a conversation with this person the next time you see him/her. Talk to the elderly lady at the supermarket. Share gardening tips with a neighbor. Offer lunch to the kid who mows your lawn. Make a plan to make a connection with them in your spare time, then give it a try and report back. | Research has found that connecting with people different from us broadens our compassion and awareness and deepens our sense of community while opening us up to new perspectives on life. When we try to take the perspective of others, we behave more admirably and generously, as it increases compassionate emotions. Perspective-taking also makes us view—and treat—others more like ourselves. According to researchers at the University of California-Berkeley, young adults from affluent backgrounds are less empathetic to the suffering of others than people whose upbringing involved some financial struggle. It’s not that they’re cold-hearted—scientists believe they simply aren’t as adept as recognizing the signals of suffering because they haven’t experienced those types of obstacles in their lives. |
| E-06 | Think of someone you know-perhaps a friend or coworker-who disagrees with you about something rather minor. Maybe they don’t understand the genius of your favorite TV show, or perhaps they can’t stand cats, and you’ve got five of them slinking | Research clearly suggests that when we empathize with others and try to see the world through other’s eyes, we’re more likely to treat them with kindness. And when we appreciate someone else’s point of view, he/she will most likely become more sympathetic and understanding of ours. Studies have found that people are generally kinder and more helpful after perspective-taking, as it increases |
| | around your home. Take a moment to think about what it’s like to be them. Why do they feel differently from you? | compassionate emotions towards the person. This exercise also leads us to view and treat others more like ourselves. Scientists found that when we actively try to understand another person’s perspective, we think of them more positively afterwards. |
| E-07 | The next time someone close to you acts in a way that hurts or upsets you, take a few moments to think about the context and try to understand what might have led to their behavior. What circumstances or events might have led them to act the way they did? Were they really trying to offend you? | This activity asks you to consider a mindset that differs from your default one, which is what perspective-taking is all about. Researchers have found that people are generally kinder and more helpful after perspective-taking, as it increases compassionate emotions towards the other person. This exercise also leads us to view and treat others more like ourselves. Scientists found that when we actively try to understand another person’s perspective, we think of them more positively afterwards. Keep in mind: It’s not too much of a stretch to empathize with someone when they act in a strange or perplexing way, but the exercise becomes more challenging when your feelings (or your ego!) are involved. And it’ll get even more difficult to empathize with someone when you’re engaged in an outright conflict with them. (Hey, we never said empathy was easy to master!) In a situation where your feelings are hurt, you may find that you feel quite differently after you take a step back and think about the context. |
| E-08 | Think of what it’s like to be someone with whom you disagree strongly about a topic that’s moderately important to you. Can you come up with reasons why they might think or feel differently? For example, maybe you’re a die-hard Giants fan in the middle of a bar filled with Pats fans. Why are they such staunch supporters? Or, think about your coworker who’s been a vegetarian for 10 years--maybe she doesn’t believe in eating meat because she had an eye-opening experience at a farm. | When it comes to empathy and understanding where others are coming from, it might not be too difficult a task when you merely have different opinions about a fairly neutral topic, like where to find the best cupcakes in town or the best singer on “The Voice”, but it’s not so easy when you disagree about something that’s pretty important to you. That’s where perspective-taking comes in. Studies have found that people are generally kinder and more helpful after this exercise, as it increases compassionate emotions towards the other person. This exercise also leads us to view and treat others more like ourselves. Scientists found that when we actively try to understand another person’s perspective, we think of them more positively afterwards. So why is this important? Being able to take another person’s point of view is crucial to making and keeping friends. Empathy fosters deep social connections, and the benefits even extend to our romantic lives: In a study of couples, people who scored higher in perspective-taking were happier in their relationships. |
| E-09 | Reflect on a recent conflict you had with someone that really hurt your feelings. For example, maybe your spouse insulted you during an argument about the family’s spending | This activity asks you to consider a mindset that differs from your own, which is what perspective-taking is all about. You’ve probably tried empathizing with a friend or coworker who’s behaved strangely or even in a hurtful manner—but now you’re ready to practice empathy during |
| | habits, or your sister made a cutting remark during your last get-together. Try to gain some insight into where they were coming from. Why were they at odds with you? What was their perspective? | a conflict or argument, which, we admit, can be quite a challenge for most folks! In a situation where your emotions are stirred up and your feelings are hurt, you may find that you feel quite differently after you take a step back, mentally remove yourself from the situation, and think about the context. Researchers have found that people are generally kinder and more helpful after perspective-taking, as it increases compassionate emotions towards the other person. This exercise also leads us to view and treat others more like ourselves. Scientists found that when we actively try to understand another person’s perspective, we think of them more positively afterwards. |
| E-10 | Think of something you can do in your spare time that will allow you to improve the life of someone very different from yourself, whether it’s tutoring a child or spending time with an elderly resident at a nursing home. Then, do it! | Research shows that helping others distracts us from our own thoughts and concerns. Anxiety and stress involve a high degree of focus on self, so focusing on the needs of others helps us shift our thinking and boosts our self-esteem. In a 2015 study, researchers found that small acts of kindness like opening a door or simply asking someone if they needed help buffered people against everyday stress and made them feel happier. Once you’ve started helping someone, don’t be surprised if it feels pretty, well, natural. In studies of human cooperation, David Rand of Harvard found that the first impulse of adults is to help others, and the same instinct to help others achieve goals has been found in infants (and even chimpanzees and rats). A multi-year study from researchers at three universities also found that helping others may not only help buffer the negative effects of stress, but lengthen our lives. Lastly, acts of kindness are contagious. Research shows that when people benefit from kindness they “pay it forward” by helping others, creating a spiral effect for more and more acts of kindness. |
| E-11 | Imagine what it’d be like for one of your close friends, your spouse, or one of your family members if you disappeared, or had never existed in the first place. In what ways would their lives be worse? What would they miss the most about you? | When you imagine stepping outside of yourself and into the shoes of someone close to you, you have the opportunity to experience an entirely new perspective on life—and your role in it. Scientists have found that when people participate in a perspective-taking exercise like this one, they tend to act with more kindness and helpfulness, as it increases compassionate emotions towards the person whose perspective has been taken. This exercise also leads us to view and treat others more like ourselves. Scientists found that when we actively try to understand another person’s perspective, we think of them more positively afterwards. So why is this important? Being able to take another person’s point of view is crucial to making and keeping friends. Empathy fosters deep social connections, and the benefits even extend to our romantic lives: In a study of couples, people who scored higher in |
| | | perspective-taking were happier in their relationships. |
| E-12 | Think of what it’s like to be someone (real or hypothetical) with whom you vehemently disagree about something very important to you—for example, over a hot-button political or religious issue. What challenges do they face? Why do they feel as they do? We know it’s not easy, but in your mind, try to treat this person with sympathy, understanding, and acceptance. | At this point, you’re practically a pro at walking in other people’s shoes. You’ll probably agree that it’s not too difficult a feat when you simply think differently about a fairly neutral topic, but empathizing with someone who has opinions you violently disagree with can seem practically impossible. However, we know you can do it with a little effort in perspective-taking! Studies have found that people feel more compassionate emotions and behave more admirably after this exercise, as it increases compassionate emotions towards the other person. This exercise also leads us to view and treat others more like ourselves. Scientists found that when we actively try to understand another person’s perspective, we think of them more positively afterwards. So why is this important? Being able to take another person’s point of view is crucial to making and keeping friends. Empathy fosters deep social connections, and the benefits even extend to our romantic lives: In a study of couples, people who scored higher in perspective-taking were happier in their relationships. |
| E-13 | Creating the intention to seek out and create micro-moments of loving connection can be a tool for elevating your well-being. Today, aim for three micro-moments filled with warmth, respect and goodwill with a family member, friend, colleague, or even someone you don’t know that well. Freely offer your attention and eye contact as you talk or touch. Later, lightly reflect on whether these exchanges brought you a feeling of positivity resonance: a back-and-forth reverberation of positive energy. | Experiences of positive emotions do more than feel good. Research shows they also “do good” in that they can broaden your outlook and build your resilience and resourcefulness - even your physical health. New evidence suggests that this may be especially true for the positive emotions that you share with others in real-time, face-to-face interactions. Micro-moments of positive connection allow positive emotions to reverberate between people, creating a powerful resonance of good feeling and goodwill. Barbara Fredrickson, Ph.D., Kenan Distinguished Professor of Psychology and Neuroscience at the University of North Carolina at Chapel Hill, has been investigating the various benefits of positive emotions for 25 years and her latest book, Love 2.0, spotlights the benefits of what she calls “positivity resonance.” Experimental evidence from Fredrickson and her team suggests that positivity resonance may be the active ingredient that links good feelings to good health. |
| G-01 | Buy a little something for anyone you want and surprise him/her with it. Ideally, it would be something that will spark some interaction with them (if you buy someone a book, you can discuss it together; if you buy someone a cup of coffee, you can chat while drinking.) | Acts of kindness are contagious. Research shows that when people benefit from kindness they “pay it forward” by helping others, creating a spiral effect for more and more acts of kindness. But when you give to others, no one benefits as much as you. One study gave participants a sum of money and then asked some of them to spend the money on themselves. The others were told to spend it on someone else. The people |
| | | who spent money on someone else were significantly happier than those who spent the money on themselves. |
| G-02 | Most of us do nice things for other people without really thinking about it. Today you’re going to do something nice for 5 different people. Your “act of kindness” can be anything — call your mom, hold the door for a stranger, compliment a co-worker - or use your imagination and stretch yourself. Buy groceries for an elderly neighbor or help a co-worker with a project. Hold a door open for someone or add coins to an expiring parking meter. You decide! You might be surprised by how much it cheers you up. | Have you ever experienced a “helper’s high” while assisting a neighbor, volunteering, or donating goods to others? Or maybe while lending an ear to a friend, or passing on your skills to someone else? Dr. Sonja Lyubomirsky, psychology professor at University of California, Riverside, asked students to commit five random acts of kindness a week for six weeks. One group had to perform five kind acts in a single day for each of those weeks, whereas the other group could spread their kind acts out over the course of the week for six weeks. Guess which group showed a 42% increase in happiness? The students who had to perform five acts of kindness in one day. That’s why doing five acts of kindness in one day is so effective. In order to get it done, we have to plan ahead, refocus our priorities and shift our thinking. Studies also show that regularly performing kind acts makes people happy for extended periods of time, but that mixing up the types of kind acts is key. To maintain the happiness boosting effects of the act, people had to vary the types of acts they did; otherwise it became a tedious experience. All the more reason to be creative! On top of it all, behavior that distracts us from our own problems also boosts our self-esteem and gives us a sense of purpose. |
| G-03 | What nice thing did you do (or are you planning to do) for someone today? Whether it’s running an errand for a busy friend, baking cookies for a neighbor or mentoring a child, record it here. You can repeat (and report!) this activity as often as you’d like. | Research shows helping others distracts us from our thoughts and concerns. Anxiety and stress involve a high degree of focus on self, so attending to the needs of others helps us shift our thinking and boosts our self-esteem. In a 2015 study, researchers found that small acts of kindness like opening a door or simply asking someone if they needed help buffered people against everyday stress and made them feel happier, and a 2016 study found that being kind to others gives us a bigger wellbeing boost than being kind to ourselves. Studies also show that regularly performing kind acts makes people happy for extended periods of time, but that mixing up the types of kind acts is key. To maintain the happiness boosting effects of the act, people had to vary the types of acts they did; otherwise it became a tedious experience. All the more reason to be creative! |
| G-05 | Spend an hour devoting yourself to an activity that you find meaningful. You could select a skill that’s valuable to you and invest an hour developing it (for example, getting better at playing the guitar) or try something new. | According to research, we find the most gratification from experiences we’ve chosen for ourselves that fully engage and absorb us, that satisfy and fulfill us and that allow us to connect us with others. Our minds want to be fully engaged in activities that are meaningful. Learning new things, pursuing hobbies, being creative with our leisure time - |
| | (Make a plan to start a new hobby or take a class, or listen to live music or read a new book.) Just plan it and do it. | these are the activities of a seeking mindset. One recent study by Chancellor and Lyubomirsky suggested “the most satisfying pursuits should involve learning new skills (e.g. mastering a new instrument or learning a foreign language), spending time with others (e.g. taking out one’s family to dinner or having coffee with a friend), or doing something good for someone else (e.g. buying holidaydecorations for an elderly neighbor or sending a carepackage to a sick friend).” |
| G-06 | Your choice. Come up with an experience you find meaningful and ask someone to share it with you - on your dime. (Remember, it doesn’t have to be expensive to be worthwhile!) | Life experiences become part of who we are. They’re woven into our memories and they shape our identity. Research shows that people who spend money on experiences are happier than those who spend money on material possessions. And it’s true across most demographic categories: male or female, liberal or conservative, high or low income and religious preferences. The study also showed that people who spent money on a series of smaller events were happier than those who spent money on one big event. In other words, you’ll be happier if you spread out your positive experiences rather than spending money on “peak” experience. When you share the experience with others, you get even more bang for your buck. Shared experiences strengthen bonds between friends, couples, and families. And according to scientists, the one thing that sets apart the happiest 10 percent of the population from everyone else is (drumroll)... the strength of their social bonds. Yale researchers also found that when people pay attention to the same pleasant thing, whether it’s a song, a dessert, or a scenic view, the experience is much more pleasurable. In other words, we get more joy out of experiences when they’re shared than we do alone. |
| G-07 | When something good happens to someone close to you, make a point of helping him or her prolong the excitement. Tell them how much they deserve it, ask lots of questions, encourage them to commemorate the occasion—or even plan something to help them celebrate with you and others! | Most of us already share the big milestones in our lives with others - engagements, births, promotions, marriage. Research suggests that the sharing of good fortune actually contributes to our well-being, and that the benefits of sharing our good fortune can extend beyond the happiness from the event itself! Perhaps less well known is the power of “capitalization”: When we show enthusiasm for others’ goals and the milestones they are hitting along the way, we are capitalizing, or prolonging our positive feelings about something important to us. Studies show that amplifying the pleasure of a good situation that involves others contributes to an upward spiral effect of positive emotion that boosts happiness. Shelly Gable’s research also shows that when we respond to, and share, other people’s good news, they feel more understood, validated and cared for. This type of sharing enhances relationship quality, and increases happiness and |
| | | life satisfaction. |
| G-08 | Identify something that someone close to you does that often annoys or upsets you. See if you can become more forgiving about that particular behavior. Can you be more understanding about why they do it? Can you be more patient with them when it occurs? | Forgiving someone for a slight or an annoyance is often easier than it sounds. However, people have an easier time forgiving those whom they feel close to, like friends and family, or people for whom they feel empathy. As difficult as forgiveness can be, the overwhelming benefits are worth it from both a physical and emotional standpoint: Researchers have found that people who tend to forgive are less anxious, less depressed and less hostile. They also have higher levels of well-being, positive emotion, and are more satisfied with their lives in general. In studies measuring cardiovascular reactivity, people who imagined forgiving a transgression they’ve experienced had lower blood pressure and heart rate than participants asked to hold a grudge. Finally, those who readily forgive have a reduced risk for nicotine dependence and substance abuse. Isn’t that enough to convince you to be more forgiving? We think so. |
| G-09 | Plan an activity or excursion with one or more members of your inner circle-perhaps a group of your best friends, or your partner or child. It doesn’t need to be expensive-in fact, it might not cost any money at all-but make sure the activity emphasizes new experiences that will involve a lot of interaction with each other. | One of the most precious things you can give someone is your own time. You’ll experience a bigger mood boost after doing this activity if you take your time without employing any shortcuts. So when you’re planning your activity, select a day when you’re not feeling rushed or guilty about leaving other duties undone. In 2002, researchers found that people who spent less time alone and more time talking to others tended to be happier. Ten years later, a different team showed how to maximize the feel-good power of conversation: Spend less time on chitchat. The happiest people, it turns out, have a third as much small talk and twice as many meaningful conversations as the least happy people. Yale researchers also found that when people pay attention to the same pleasant thing, whether it’s a song, a dessert, or a scenic view, the experience is much more pleasurable. In other words, we get more joy out of experiences when they’re shared than we do alone. |
| G-10 | Think of a grudge you’re still holding against someone for something they did that offended you. It may not be easy, but perform a forgiveness exercise to see if you can let go of your hard feelings. Think of the energy that goes into holding on to this grudge, and assess whether there are others in your life that are being affected by your | We feel generous when we forgive someone for a slight or an annoyance, but it’s more difficult to forgive someone who’s truly offended us. As hard as it is, the overwhelming benefits are worth it from both a physical and emotional standpoint: Researchers have found that people who tend to forgive are less anxious, less depressed and less hostile. They also have higher levels of well-being, positive emotion, and are more satisfied with their lives in general. In studies measuring cardiovascular reactivity, people who imagined forgiving a transgression they’ve experienced had lower blood pressure |
| | inability to forgive this person. Perhaps you can even think of a few benefits that arose as a result of the offense. | and heart rate than participants asked to hold a grudge. Finally, those who readily forgive have a reduced risk for nicotine dependence and substance abuse. To help facilitate forgiveness, it’s useful to look for ways a personal transgression may have benefited you in some way. In a writing intervention by McCullough, Root, and Cohen, people who spent 20 minutes writing about the benefits that arose from a transgression reported more forgiveness than people in a control group who were asked to write about an unrelated topic or an unpleasant part of the transgression. |
| G-11 | Identify a cause that you care about and volunteer your time to furthering it in some tangible way. If education is important to you, consider donating tutoring students or helping high school students with their college admission essays. If you’re an animal lover, spend Saturday mornings walking dogs from the local shelter. | Volunteering and helping others distracts us from our own thoughts and concerns. Depression, anxiety and stress involve a high degree of focus on ourselves, so when we have a positive impact on someone else, we boost our mood and self-esteem, and give our life a greater sense of purpose. A study conducted by the University of British Columbia and Harvard Business School suggests that kindness and happiness form what’s called a positive feedback loop. When we do something nice, we feel happier. And the happier we feel, the more likely we’ll perform another kind act. Volunteering is also good for your health: Researchers have found that helping others is associated with reduced stress and a longer life! |
| G-12 | What cause is important to you? Make a charitable contribution (you don’t have to break the bank!) that will allow you to directly witness a person (or group of people) benefiting from your generosity. For example, maybe your donation to a local community garden allows everyone in the neighborhood to have easy access to fresh vegetables. Or, you chip in for an operation that a friend’s son desperately needs but can’t afford. | A study conducted by the University of British Columbia and Harvard Business School suggests that giving and happiness form what’s called a positive feedback loop. Giving to charity makes us happier, and when we’re happier, we give more. And believe it or not, they also found that people actually feel wealthier when they give money away! Similarly, researchers gave participants a sum of money and then asked some of them to spend the money on themselves. The others were told to spend it on someone else. The people who spent money on someone else were significantly happier than those who spent the money on themselves. When we can see the reactions of those we’ve helped, the effect is even more powerful. In a national survey of 3,300 participants, volunteers who witnessed the reactions of the people they helped reported increased self-esteem, less stress, and a greater sense of the “helper’s high.” And neuroscience backs this up: In a brain imaging study conducted at the Washington University School of Medicine, scientists found that the reward centers of the brain lit up when participants anonymously made a charitable donation—these are the same parts of the brain that light up when people experience pleasure, like money, sex, or a really decadent ice cream sundae. |
| R-01 | Good morning, sunshine! Time to look at the bright side. Literally. This week, aim to wake up at the same time each morning (resist the urge to snooze the alarm!), and get some sunlight as soon as you can. Open your curtains and let the light fill your room, go for a walk, or simply sit outside in the sun with your favorite wake-up beverage. What’s the bright idea behind this? Exposure to light when you first wake up will help reset your circadian clock and improve your energy levels during the day. It will also help your body get the message that nighttime is for sleeping. (Option:) Create a positive wake-up routine that includes activities you love. For example, you could play upbeat music, use a shower product with a scent you find energizing, drink really good coffee, or take a moment to think of what you’re grateful for and what you want to accomplish that day. Before long, you may find yourself excited to wake up every morning because you get to do all these awesome things! | One of the systems in the brain that controls when we sleep and when we’re awake is called the internal circadian clock. This clock is highly sensitive to light, and research shows that bright light in the morning can help wake you up and have a positive effect on your energy levels and hormone production. Exposing yourself to light early in the morning can also help improve your sleep at night by helping your circadian clock know when to start winding down. The benefit of purposely exposing yourself to light is supported by a whole lot of science. Research shows that a regular schedule is good for your circadian rhythm, which is what wakes you up at the right time each day, as long as you keep your routine consistent. If you go to bed and wake up at a different time each day, the circadian rhythm will get confused and you’ll be awake or sleepy at all the wrong times. The other benefit of a regular schedule is that it helps your brain know what to expect and to make sure you sleep deeply. Your brain also anticipates when you want to wake up and releases chemicals, including cortisol, to help you wake up and feel awake. If you wake up too early or too late, you won’t get this benefit. Having a fresh and happy morning routine can also tell your body when it’s time to feel awake and energized. If you wake up excited and ready to go, you perform better throughout the day and feel less disoriented when you wake up. |
| R-02 | Autogenic training is a relaxation technique that can help restore balance to mind and body. It offers a wide range of benefits, from reducing insomnia to improving sleep patterns. This guided practice is designed to support you to fall asleep faster and wake up feeling more energized. Find a quiet place free of distractions and try this brief guided autogenic training exercise to help you ease into a rest and relaxation mindset. | Autogenic training is similar to meditation in that it helps you feel calm. And calming your mind to pay attention to the sensations and rhythms in your body has a ton of benefits. It can help you to function better and feel less anxious during the day and also wind down more easily and sleep better at night. This relaxation technique typically involves a series of statements about the heaviness or warmth of various places in the body. Over time, you may find that it takes less and less time to bring about a peaceful state of mind. |
| R-03 | Step away from your work or must-dos one hour before your bedtime routine, and turn off your electronic devices at least 30 minutes before bedtime. Use this time to sit with yourself and think through your day. What were the highlights? Did you inch closer toward a goal or follow through on something that was | Consistently getting into bed feeling relaxed and prepared for sleep can have a profound impact on sleep health in both the short and long term. Having a predictable, pleasant bedtime routine lets you feel your natural sleep pressure. If you work out, watch an intense movie, or have a lot of social interaction before bed, however, chances are you’re going to feel wired and not at all ready for sleep. Give yourself space between your daily activities and bedtime to slow down and signal to your body |
| | important to you? Did you connect well with the people around you? Make a list of things that were impactful that day and a list of things to think about or deal with tomorrow so you don’t lie awake thinking about them. | that it’s time for bed, and then sleep should come more naturally. Try to give yourself permission to let go of negative thoughts and worries as well. Repetitive negative thinking, doing activities, and working close to bedtime can result in a longer period of time needed to fall asleep. Bright lights from electronics can also delay sleep, so it’s best to turn them off a few hours before you go to bed and keep them out of the bedroom. |
| R-04 | (Several task variations can fall under this activity, or they can be combined:) Task 1: (You Decide How) If you’re having trouble falling asleep at night and it’s been 20 minutes or more, put on a comfy robe or blanket, keep the lights low, and go do something relaxing. This could be reading, writing in a journal, or petting your dog or cat. The point is to change your environment so that you are practicing stimulus control—meaning sending yourself the correct messages, like bed is for sleeping, not for being awake. Do this every night and notice how soon you are able to fall asleep faster. Task 2: Turn Back Time: Clocks are a basic necessity throughout our day, but when it comes to sleep, it’s best to clock out. Try moving your clock out of sight at bedtime, and if you have to wake up at a certain time in the morning, set an alarm and don’t look at your clock until it goes off. If you wake up in the night, don’t worry about what time it is, and don’t even check. Just take some relaxing deep breaths and go back to sleep, however long that might take. Task 3: Time and Again: Try to wake up at the same time every day, even on your days off. If your alarm for work normally goes off at 6:30, for example, get up at 6:30 on the weekends, too, but do something enjoyable. Make coffee, go for a walk or to sunrise yoga, or read a book and then plan out your day. You can turn | Many people who don’t get enough sleep prioritize other things instead, like work, socializing, or relaxing. Millions of others suffer from insomnia, a very common sleep disorder where people can’t fall asleep or stay asleep, no matter how hard they try. Whatever the reason, there are many strategies we can use to help us get all the sleep we need. One way is to increase sleep efficiency. Sleep efficiency refers to how well you’re using your time in bed. People with a low sleep efficiency are spending a lot of time in bed not sleeping. If you decrease the amount of non-sleeping time you spend in bed, you might just become a more efficient sleeper. Helping yourself to fall asleep sooner by getting enough exercise during the day, eliminating caffeine, and putting devices away early in the evening can also increase your sleep efficiency. Stimulus control is also important. Stimulus control was developed based on the core principles of classical conditioning. The idea is that your body can learn a response to being in a specific situation, especially if that response is consistently paired with that situation. For example, if the bed is for sleep only, getting into bed will trigger a sleep response. However, when other activities occur in bed, like working on your laptop, sleep is no longer the triggered response. Instead, no specific response is triggered, or an alternative one is (such as thinking about work). Stimulus control is a powerful technique and an effective way to improve sleep, reduce insomnia symptoms, and improve sleep efficiency. Why is it so important to stick to a regular bedtime and wakeup schedule? A 2019 study found that getting different amounts of sleep each night can put you at higher risk for obesity, hypertension, high cholesterol, and other metabolic disorders. [Clock tyranny:] When people wake in the middle of the night, one of the first things they typically do is to check the time. But this is actually making things worse for yourself because frequent clock-checking can interfere with sleep. Try to make the clock (mostly) inaccessible, to reduce the temptation to check it. Maybe put your phone in a drawer or turn the bedside clock away from you. This way, you won’t get that nocturnal rush of brain activity that could make it |
| | those early morning hours into some epic “you” time! Task 4: Night Owl or Early Bird?: Do you ever describe yourself in terms of when you’re at your best, such as “I’m a night owl” or “I’m an early bird”? It turns out there’s something to it, and it’s actually beneficial to just roll with it. If your eyes get heavy at 9:30pm, for example, great! It’s time for bed. If you’re the type that just hits their stride in the evening and you groove until midnight, great! Do your thing and head to bed when you’re tried. Try matching your sleep and wake times to your natural preference, and you’ll be more in sync with your natural self. Task 5: There’s a Nap for That: Guess what: napping is allowed! If you feel yourself getting low on energy in the middle of the day, find a comfortable place to lie down if you can, pull a soft blanket over you, and nap away! A brief nap early in the day can be a great way to boost your energy and reduce fatigue. Try to keep your midday zzzz to under 40 minutes and lie down as close to midday as possible. (The later it is, the more trouble you might have falling asleep at bedtime.) | more difficult to return to sleep. [Wake up at same time each day:] If you can always wake up within about one hour of your usual wake time every day, rather than waking up at all different times, you’ll sleep much better at night and feel better during the day. Why? Because your brain likes routine, and it’s a fabulous host that gets things all ready for you before you wake and before you sleep. Think of it this way: Your brain is like that friend who throws a fantastic dinner party and has all the food and drinks set out for you before you arrive so you can have a great time while you’re there. But instead of food and drinks, your brain sets out super helpful chemicals, like cortisol to help you wake up and feel awake, or melatonin to help you wind down and feel sleepy. If you show up to the dinner party too early or too late, the host won’t be ready for you and the party (a.k.a. how you feel that day) will flop. [Night owl or early bird:] Science strongly supports that people are different when it comes to their best time of day. Those who think faster and feel better in the evening are often called “night owls” while those who do best in the morning are called “early birds.” Night owls can survive as early birds, but they tend not to flourish. If your best performance is in the evening, try to adapt your schedule so you can be awake at those times. [Napping:] A relatively brief nap can improve physical performance, reduce fatigue, and improve mental performance. Naps can also help improve learning and memory. The length of the nap doesn’t seem to matter, as long as it’s kept to less than an hour and is early in the day. Too much napping, however, is a no-no: it will likely throw off your internal clock and make your sleeplessness worse the next night! |
| R-05 | You should probably sit down for this. Actually, scratch that, you might want to stand up for this news: sitting for long periods is really bad for you. Most of us do it because we have desk jobs, so it makes sense how we got here. But we need to break the habit and take a stand. Try this: While sitting at work or at home, whether you’re watching TV, listening to music, reading or performing desk work, make a point to stand up for 3 to 5 minutes every hour. You can continue watching, listening, or reading/working while standing, to minimize the interruption to your activity. | You should probably sit down for this. Actually, scratch that, you might want to stand up for this news: sitting for long periods is really bad for you. Most of us do it because we have desk jobs, so it makes sense how we got here. But we need to break the habit and take a stand. Try this: While sitting at work or at home, whether you’re watching TV, listening to music, reading or performing desk work, make a point to stand up for 3 to 5 minutes every hour. You can continue watching, listening, or reading/working while standing, to minimize the interruption to your activity. |
| R-06 | To calm any stress and anxiety you might be feeling, connect with your breath by performing diaphragmatic breathing (DB) exercises throughout the day. This means breathing deeply so that when you inhale, your belly expands—rather than your chest-allowing you to breathe more fully. Notice how you’re feeling before the exercise, and then check in again after. The simplest technique involves inhaling through your nose for a count of 3 seconds, holding for 4 seconds, and exhaling for 3 seconds. | Performing diaphragmatic breathing has many benefits, including reducing anxiety, stress, and cortisol levels (which is the hormone we release under stress). It also reduces heart rate and breathing rate and increases melatonin levels, which help us sleep. What’s more, diaphragmatic breathing has been shown to increase our ability to pay attention and also help reduce negative thoughts, which are often the result of stress and anxiety. Talk about a powerful defense mechanism! |
| R-07 | When it comes to exercise, every little bit helps. And you can get your little bits in more easily than you think. For example, set an alarm once in the morning between 9am and 12pm and once again between 1pm and 5pm during the workday. When your alarm goes off stand up from your desk and go for a brief 3-5 minute walk. (If you’re able to, you can lengthen the time of your walk or recruit a “walking buddy” to do it with you.) [More difficult version:] Move on the Job: If you want more of a challenge, try this: During the day, incorporate two brief walking excursions into your routine. First, you could park farther away from your office than you normally do and take the extra steps into work. For your second excursion, walk to a co-worker in another building or on a different floor to stay hi, instead of calling them. [Advanced versions:] Track Your Moves: Track your steps using a pedometer, accelerometer, smartwatch, or smart phone, and set a goal of steps each day. The number of steps can range from an average of 2775 or higher. You can also run, cycle, or get your heart pumping in any other way you choose. | A recent study showed that sporadic walking or moderate-to-vigorous physical activity of any duration, including bouts as short as 5 minutes, improves our overall health and helps us live longer. You can do 5 minutes! And a 30-minute morning walk was found to be as powerful as medication when it came to lowering the blood pressure of sedentary older adults. Walking too boring for you? Researchers also found that swapping even half an hour of sitting for some type of physical activity of any intensity can reduce your risk of early death by 35 percent. Even those least inclined to be active and at the greatest risk for developing chronic disease can benefit from performing short bouts (5-10 minutes) of moderate walking to improve health outcomes. The benefits of walking are pretty significant, and you don’t have to do as much as you think. For example, one study sought to find the impact of a 100-day, 10,000-step program in 1,963 people, measuring signs of depression, anxiety and stress, as well as general well-being. At the end of the 100 days, all measures of mental health were improved regardless of how many steps a person walked over the 100-day period. Interestingly, the disparity between steps was pretty great, and all with the same outcome: some people walked an average of 2,775 steps, while others walked as many as 112,831 steps. This study confirms that you don’t need to walk 10,000 steps a day, as many people believe, to get the mental health benefits of walking. ~R-19 A 2019 study of over 16,000 senior women also found that those who walked just a moderate amount (an average of just under 4,400 steps a day) were 41 percent less likely to die over the next four years than women who walked around 2,700 steps a day. Aerobic exercise is beneficial for your brain, too—a recent study found that adults assigned to a 6-month aerobic training program significantly improved their cognition and executive function. |
| R-08 | Stretching is like the dessert portion of fitness routines. For 5 to 7 minutes each day, have your dessert by performing a simple series of large-muscle, whole-body stretches, using traditional stretches found in yoga, qigong, and tai chi. These stretches will get your blood flowing into places that may have been blocked by tight muscles. | Stretching can be done almost anywhere, and it is surprisingly good for you. Moreover, the benefits aren’t only physical: for example, 30 minutes of stretching exercises can enhance your mood and make you think a little faster and even improve your reaction time. One study showed that a 12-week yoga intervention resulted in increased brain thalamic GABA levels, which improves our mood and decreases anxiety levels. Yoga stretches also help to regulate our hormones. |
| R-09 | Engage your core! These are three of our favorites, which you can—and should—do several times a week. The first exercise is cat-cow performed on your hands and knees with your back straight, hands under your shoulders, and knees under your hips. Begin by arching your back up and hold for 3 seconds, and then let your back/abdomen sag to the floor and hold for 3 seconds. Perform 5 sets. The second exercise starts in the same cat-cow position (hands and knees on floor). Begin by extending your opposite upper and lower limbs straight out (for example, left leg behind, right arm in front); maintain for 30 seconds and switch sides. The third exercise is a spine twist It begins with kneeling on a pillow with your arms extended out to your sides. Rotate your trunk, head and arms to one direction and hold for 30 seconds and then rotate to the other side. Repeat 2 times. | Currently, low back pain (LBP) affects more than 80% of people at some point in their life and often results in lost wages, additional medical expenses, and a risk of developing other medical/health conditions. In the United States alone, total indirect and direct medical costs of LBP exceed $100 billion every year. A number of studies confirm that core/trunk stabilizing/balancing exercises are proven very effective for helping to treat and prevent LBP. Some of the research shows that performing these exercises for as little as a week is effective. Additionally, even in healthy adults, integrating core stability training into your exercise regimen may help to prevent injury, particularly in the lower extremities (think hips, knees, and ankles). |
| R-10 | Plan on signing up for a fitness class (including yoga, tai chi, pilates, aquatics, or ballroom dancing), ideally one that you can commit to with some co-workers, a partner, or friends. It can be first thing in the morning, at lunch, right after work, or in the evening. The important things are to get moving, do something you enjoy, and do it with others so you’re held accountable. If you find it really | A fascinating study using temptation bundling showed that when exercise is bundled with tempting audio book novels, it increased college students’ gym attendance by 51 percent! The study also showed that for people with self-control challenges, temptation bundling was especially effective by offering a low-cost solution to two common willpower problems of under engagement (not engaging in a “should” behavior like exercise, often enough) and over engagement (indulging in “want” behaviors too often). Ultimately, having the motivation to perform exercise is essential to forming a long-term habit, and this is driven |
| | challenging to exercise in general, incentivize your commitment to the class with what’s called “temptation bundling”. This involves linking an instant gratification “want” activity, such as watching the next episode of your favorite TV show, listening to your favorite podcast, reading a chapter from a book you’re loving, or receiving a massage, with a “should” behavior that provides a long-term health benefit but requires energy and willpower to complete, such as exercising. In short, do the “should” to get the “want.” For example, you could listen to your favorite album while you run on the treadmill, go to your favorite restaurant with friends after your pilates class, or listen to your audiobook on your way home from yoga. The bundle options are endless! | primarily by the self-determination theory, which focuses on personality factors and the surrounding environment of each individual. Choose your exercise companions wisely because they will greatly influence your likelihood of sticking with the exercise long term. Of course, this also influences your satisfaction and enjoyment of the exercise itself! |
| R-11 | Try boosting your workout routine a little to see what differences you notice. Combine a resistance exercise, like lifting weights, using resistance bands, or even water exercises, with your whole-body workout routine 2 to 3 times a week to increase your strength, improve your overall wellness, and enhance your thinking abilities. Does it make you feel stronger, faster, and happier? | Resistance/strength training improves not only your physical strength and performance abilities, but also your cognition and executive function. This is true for people across age groups, including older men and women. ~R-28 One study showed resistance training twice a week results in the most favorable changes to quality of life and sense of coherence (feeling optimistic and in control) among men and women 65 to 75 years old. Additional research showed that resistance training improves body composition (reduced fat mass, increased lean body mass), muscle strength, and physical function in the obese elderly, whether the individual has changed their diet or not. It can also give your mental health a boost. A meta-analysis of 33 randomized controlled trials found that resistance exercise training was associated with a significant reduction in depressive symptoms. |
| R-12 | Figure out your ideal fitness routine-you know, the one that makes you feel glowy and awesome head-to-toe afterward—and make a commitment to stick with it. This is your jam! Choosing the most appropriate fitness routine is a personal decision that takes into account preferences, goals, feasibility and time. For example, you might love how free weights, boxing, | Committing to a comprehensive fitness routine has many proven physical and psychological benefits-it gives us a feeling of accomplishment, helps us reduce stress, and promotes good sleep. In one study, people who did low, moderate, or high levels of physical activity were assessed for changes in mood and levels of brain activation of pleasure-seeking receptors. It turns out all levels of physical activity increased brain receptors of pleasure and enhanced mood improvements. In other words, regardless of the level of physical activity/exercise you perform, you will likely |
| | and spin class make you feel. Find the best times of day and the most convenient locations for you to do these favorite activities, and make it part of your daily routine. Period. Fitness and performance training are powerful: they enhance your mood and make you feel stronger, which in time will make you want to keep doing it. | feel happier. Additionally, when Yale and Oxford researchers collected data on over 1 million individuals, they found that people who exercised regularly tended to be happier. On average, regular exercisers felt bad for 35 days a year, whereas nonactive individuals felt bad for 53 days, on average. What’s more, they found that the active group felt just as happy as nonactive individuals who earned about $25,000 more a year. In other words, one has to earn a lot more to get the same happiness boost from regular exercise and sports. ~R-36 No need to overdo it, either. The same study found that when it comes to better mental well-being, three to five training sessions per week, each between 30 to 60 minutes, are ideal. People who exercised for more than three hours a day actually had lower mental health than people who weren’t particularly active. Regular exercise has also been shown to be an effective treatment for depressive disorders by itself and when combined with other therapies. Remember, there’s no one size fits all when it comes to an ideal exercise routine. What works for someone else may not work for you, so take stock of your capabilities and preferences as you craft a routine that’s ideal for you. |
| R-13 | Water is essential to life, but do you know what kind of life is waiting for you if you drink more of it? Increased water intake may lower your risk of depression and anxiety, and even improves cognitive function. Keep a water bottle filled with you wherever you go, and drink, drink, drink. In general, you should try to drink between half an ounce and an ounce of water for each pound you weigh, every day. We know. It’s a lot. But you get used to it quickly, and you can make it extra delicious by adding electrolyte tablets, or slices of lemon, lime, cucumber, or a handful of berries. | If you don’t tend to drink much water, like less than about a liter a day, doubling your water intake can significantly help improve any feelings of fatigue, confusion, thirst, or sleepiness you might have. One study showed that doing the opposite—cutting water intake in half for people who already drink enough water—had disastrous effects: it increased thirst and decreased feelings of contentedness, calmness, positivity, and vigor. Don’t do that! A related study showed that for every one percentage point increase in daily water intake, the quality of people’s diets improved drastically. Lowering your water intake, however, down to less than two glasses of water per day, may double your risk of depression and anxiety. How amazing is it that getting enough water could protect you against these conditions? |
| R-14 | Make your mornings a significant part of your self-care routine by starting your day with a nutrient-dense meal containing a high-quality source of protein and fiber. For example, eat foods like a bowl of oatmeal with a ½ cup of Greek yogurt and nuts/seeds sprinkled on top, or eggs with multigrain toast and a slice of | A simple, high-quality source of protein for breakfast, like eggs, peanut butter, or yogurt gets the day off to an energetic start and has been shown to help reduce body fat, make you eat less during the day, help you to feel less hungry overall, and may even improve your sleep quality. Regularly skipping breakfast isn’t such a great idea. A recent study among college students showed that skipping breakfast is associated with being less happy. ~R-42 Whereas in adults, eating breakfast showed a robust |
| | avocado, or a bowl of acai, chia, flax, almond butter and sliced banana, or high-protein cereal with whole grains and fresh fruit. A protein smoothie is also a delicious way to start the day and is always a hit with kids and those on the go. Try to gather the meal together the night before so it’s ready and waiting for you to assemble in the morning. Once you get into this high-protein morning habit, you may find yourself controlling your weight better, losing body fat, feeling less hungry during the day, and even sleeping better at night! | advantage for memory (particularly delayed recall), and for adolescents, tasks requiring attention, executive function, and memory were enhanced by eating breakfast (compared with skipping it). |
| R-15 | It’s amazing how much lighter and better you can feel just by altering your diet to accommodate fresh foods. Aim to incorporate into your diet five servings a day of healthy veggies and fruit, and a handful of nuts and seeds. If you’re not eating any of these things at all, start by consuming one serving of fruit, greens (veggies), and nuts during the day. A few simple strategies include 1) Making a breakfast protein smoothie with some berries and scoop of nut/seed butter. 2) Including a salad or some colorful raw or steamed veggies at lunch and dinner and 3) Having fresh-cut veggies with hummus or Greek yogurt dip for a snack. 4) Keeping nuts/seeds and dried fruit in your car or bag so you can have a handful when you need an energy boost. | The Mediterranean diet is high in fruits, nuts, greens, fish, and healthy fats and can reduce your risk for depression. No sea kelp shakes here, just tasty, healthy food to enhance your mood. A 2016 study also found that increasing your intake of fruits and vegetables is linked to greater happiness and well-being, even after adjusting for other factors. This way of eating is also great for the rest of your body. A comprehensive meta-analysis of over 2 million people showed that eating more fruits and vegetables led to reduced risk for cardiovascular disease, cancer, and mortality in general. This study also found that fewer than 5 servings of fruit and 8 servings of veggies per day increases the risk for these conditions. |
| R-16 | Healthy snacking is all about choosing foods and beverages with the lowest simple sugar content because our body and mind function better with less sugar intake. You’re in Revive mode, so choosing healthy, protein-rich, no-sugar foods and drinks are vital to nourishing your body and growing your vitality and energy. Here are a few snack hacks that might | Eating and drinking too much sugar may make you feel physically unwell in the short term and is associated with an increased risk of diseases like Type 2 diabetes and cardiometabolic diseases. High sugar intake may even lead to common mental disorders, such as depression. In fact, risk for depression is significantly elevated in men and women who consume large amounts of sugar; it elevates to almost 23 percent over 5 years! This is especially true for postmenopausal women. People who eat the right stuff, however, like the fiber, fruits, |
| | transform your afternoons: Make a small batch of hard-boiled eggs and take one with you when you go out in the morning, along with fresh sticks of carrot, peppers, beans, cucumber, and any other raw veggie you love. Pair that with some homemade hummus (it’s so fast to make!) and you have yourself a tasty, healthy snack. For your protein boosts, you can do a few things: Make homemade trailmix with nuts/seeds and dried fruit; buy some grass-fed jerky, available in most grocery stores or order online; pick up some organic, pasture-fed Greek yogurt (which tends to be lower in sugar than other yogurts) or cottage cheese and top with berries and chia or flax seeds. | and veggies experts recommend, have much lower incidents of depression. So stick to this way of eating, and you’re on the right track all around. Our snacking recommendations are also on point: One recent study showed that eating high-protein snacks throughout the day helped to satisfy hunger in overweight/obese men, which means it likely reduced their chances of overeating at meal times or turning to the wrong foods. |
| R-17 | One of the hardest things about changing your diet is the meal prep. You’re not used to cooking with those ingredients yet, and having to make something diet compliant every night gets a little daunting and tiresome. So do yourself a really big favor and feed two birds with one stone. Each time you cook a recipe at night, make enough for at least 2 to 3 meals so you have leftovers for lunch the next day or for dinner the following night. You can also pack your snacks ahead of time in “go bags” the night before so you don’t have to think too hard when you’re running late. Just remember to include fresh veggies, fruits, nuts, and proteins in bags or containers so they’re ready as you walk out the door. | Preparing your meals ahead of time is a sure way to choose foods wisely for yourself, and it can also lead to weight loss. In fact, a study of over 1,000 adults showed that those who planned meals ahead of time, such as preparing meals the night before, had a greater likelihood of weight loss compared to those who didn’t prepare meals. If you think about it, it makes sense. Eating in a restaurant or grabbing food on the go usually means high-fat and high-sugar options, and you’re not in a position to choose fuel that your body needs most. If you’re thinking about what you want to put in your body before you’re even hungry, your choices will make much more sense. |
| S-01 | Are you savoring an incredible meal, a hike through the woods, or a gorgeous sunset? Whatever it is, focus on the details, let yourself get totally | Studies show that people who often appreciate and savor what they have—their possessions, traits, relationships or accomplishments—tend to experience more happiness than people who don’t. |
| | immersed, and use all of your senses to intensify and prolong your positive experience. Upload a photo so you can savor the memory of it later! | When mindfulness becomes a habit, the neural pathways in our brains linked to positive feelings become stronger and more active, increasing the chances we will continue to feel happier in the future. And that’s just for starters. Researchers ~S-2 have also found that savoring makes us feel more grateful and hopeful, which gives us more self-confidence. And when we feel better about ourselves, we’re more open to others. People who savor are also less likely to experience depression, stress, guilt, shame and depression. |
| S-02 | Block out 10 minutes, lie down somewhere comfortable, and spend a minute focusing on your breathing. Now, imagine you’re taking a scan of your whole body, starting with your feet and moving past your torso on up to your head. Just “feel” each body part as you scan over it, then allow it to fade and move on. Dismiss any thoughts that stray from your focus. | A 2018 study published in the journal Psychosomatic Medicine found that body scan mediation decreases stress and increases levels of mindfulness. In addition, several studies have shown that when practiced regularly, meditation has the potential to help lower high blood pressure and lessen chronic pain, anxiety and depression. It can also alter the regions of the brain associated with memory, self-awareness and compassion. In a study conducted at the Massachusetts General Hospital, participants attended weekly 2.5-hour group meetings in which they practiced mindfulness meditation. At the end of the 8 weeks, the meditators felt more capable of acting with awareness, observing the world around them and remaining nonjudgmental. So how exactly does meditation reduce anxiety? In a 2013 study, scientists at Wake Forest identified the brain functions involved in the process. While meditating, participants showed greater brain activity in the ventromedial prefrontal cortex, the area that controls worrying. And when activity increased in the anterior cingulate cortex (the part of the brain that controls thinking and emotions), anxiety levels also decreased. The best part, at least for beginner meditators? You don’t have to sit for 30 minutes to reap the benefits: According to the lead author of the study, Fadel Zeidan, Ph.D, “just a few minutes of mindfulness meditation can help reduce normal everyday anxiety.” |
| S-03 | Come up with something you can enjoy doing with someone else, and savor your experience, then discuss it afterwards. It could be a shared meal, a walk through a beautiful park, or a piece of good news you have received. Be in the moment, enjoy the present and be mindful of everyone’s joy. Add a photo too! | In a study about savoring strategies, researchers found that a savoring strategy called capitalization, or sharing, enhances our life satisfaction and increases our everyday happiness, over and above the impact of the positive event itself. They say the best things in life are meant to be shared, and recent research proves this to be true! Yale researchers recently found that when people pay attention to the same pleasant thing, whether it’s a song, a dessert, or a scenic view, the experience is much more pleasurable. In other words, we get more joy out of experiences when they’re shared than we do alone. |
| S-04 | Today, revisit a good memory from your past and take the time to savor it. Reminisce about a happy childhood event, a great vacation, a joyful family event, a personal victory, a rewarding accomplishment. Use your senses to put yourself there. Think about how it looks, smells, tastes. How do you feel? Report back and describe your savoring experience. Add a photo too! | Dr. Fred Bryant (who coined the term “savoring”) says that savoring can be “time-shifted,” meaning it can be enjoyed in the past, present and future. Some like to savor the past through reminiscing, others savor the future through the anticipation of things to come, and others enjoy just being in the present. Through his research, Dr. Bryant has also confirmed that savoring is beneficial to our health and happiness. People who regularly and frequently savor are happier, less depressed, more optimistic and more satisfied with life in general. Each form of savoring has its own benefits. People who savor the present are less prone to depression, have less stress, guilt, and shame. Those who savor the future (anticipation) are more optimistic. And people who savor the past (reminiscing) are best able to buffer stress. In fact, a 2017 study conducted at Rutgers University found that recalling positive memories could combat acute stress at a physical level. Brain scans showed that it was associated with increased activity in prefrontal brain regions associated with emotion regulation and cognitive control—the same regions suppressed by acute stress—as well as in the regions associated with “reward” processing. |
| S-05 | When faced with a difficult decision or an upcoming challenge, we often overthink or over-complicate the situation to the point where we end up doing nothing. So next time you start looping your negative tapes, create a plan to distract yourself or re-focus yourself on the task at hand. It can range from reciting a mantra to scheduling a block of “worry time” each day, to wearing a rubber band on your wrist that you snap every time you catch yourself getting lost in worries— anything that will help you to “reset” your brain and derail overthinking. | Breathe a sigh of relief: Psychologist and author Robert Leahy, Ph.D., of Weill Cornell Medical School, found that 85% of the stuff we worry about have positive or neutral outcomes. And even when our worries do become a reality, about 80% of us say we handled the outcome better than we thought we would. Research by Matt Killingsworth suggests that a wandering mind is an unhappy mind—people are less happy when their minds are wandering and distracted than when they’re not— especially if they’re thinking about something unpleasant or even neutral. (Thinking about a pleasant topic other than their current activity had no effect on happiness one way or the other.) Mind-wandering also occurs with higher frequency on tasks that don’t demand our full attention. These activities are derived from Mindfulness-Based Stress Reduction exercises, which range from mindfulness meditation to being mindful during stressful situations. When practiced regularly, they can improve coping with distress and disability in our day to day, as well as under more extraordinary conditions of serious stress. How? They help us focus on what’s happening in the present moment, rather than what happened in the past, or what might happen in the future. These activities also allow us to recognize (and distance ourselves from) a distorted thought such as “Everything’s going to go wrong.” Recognize it as simply that—a thought—and then let it go. |
| S-06 | Sit with your spine upright but not stiff, keeping your torso centered and balanced. Feel your body from the inside. Pay attention to your breathing. The past is over and the future isn’t here yet. Just settle into the present, the only moment where we’re truly living. Beginning meditators: Don’t worry when your attention wanders. Start with 5 or 10 minutes. And keep in mind: This is something you can do whenever you have a few minutes of free time. | Studies show that meditation can lower high blood pressure and lessen chronic pain, anxiety and depression. Brain imaging studies show that meditation actually alters regions of the brain associated with memory, self-awareness and compassion. Beginner? No problem! Researchers at the University of Wisconsin-Madison found that even beginners were able to increase activity in the brain region associated with positive thoughts, although experienced meditators showed a greater level of activity. In a study at Massachusetts General Hospital, participants attended weekly 2.5-hour group meetings in which they practiced mindfulness meditation. At the end of the 8 weeks, the meditators felt more capable of acting with awareness, observing the world around them and remaining nonjudgmental. So how exactly does meditation reduce anxiety? In a 2013 study, scientists at Wake Forest identified the brain functions involved in the process. While meditating, participants showed greater brain activity in the ventromedial prefrontal cortex, the area that controls worrying. And when activity increased in the anterior cingulate cortex (the part of the brain that controls thinking and emotions), anxiety levels also decreased. The best part, at least for beginner meditators? You don’t have to sit for 30 minutes to reap the benefits: According to the lead author of the study, Fadel Zeidan, Ph.D, “just a few minutes of mindfulness meditation can help reduce normal everyday anxiety.” |
| S-07 | Choose a low-energy physical routine, such as yoga or gentle stretching, that takes about 20 minutes to complete. Ideally, it should be something you don’t need to think about too much. As you go through each pose or stretch, pay close attention to the effect it has on your body. Hold the stretch, focusing your attention on every place where you feel something in your body, one place at a time. What’s the sensation like? Repeat this for each pose. | Being mindful requires you to be aware of physical sensations, perceptions, thoughts and imagery. Numerous studies have shown that mindfulness meditation, especially when practiced regularly, can improve general coping with stresses from everyday life, as well as under more extraordinary conditions. According to studies, meditation can also lower high blood pressure and lessen chronic pain, anxiety and depression. It alters regions of the brain associated with memory, self-awareness and compassion. So how exactly does meditation reduce anxiety? In a 2013 study, scientists at Wake Forest identified the brain functions involved in the process. While meditating, participants showed greater brain activity in the ventromedial prefrontal cortex, the area that controls worrying. And when activity increased in the anterior cingulate cortex (the part of the brain that controls thinking and emotions), anxiety levels also decreased. The best part, at least for beginner meditators? The benefits happen quickly: According to the lead author of the study, Fadel Zeidan, Ph.D, “just a few minutes of mindfulness meditation can help reduce normal everyday anxiety.” |
| S-08 | Deliberately arrange a day of leisure. Fill your day with different types of activities to savor—food, music, a beautiful walk, or a visit to a museum—and savor each activity using techniques you’ve learned in previous levels, from mindfulness to capitalization to paying attention to the details. | According to researchers Dr. Fred Bryant and Joseph Veroff, savoring involves noticing and appreciating the positive things all around us. Savoring is the positive counterpart to coping. It’s about much more than mere pleasure - it also involves mindfulness and “conscious attention to the experience of pleasure.” Researchers found that savoring makes us feel more grateful and hopeful, which gives us more self-confidence. And when we feel better about ourselves, we’re more open to others. People who savor are also less likely to experience stress, guilt, shame and depression. In one study, researchers found that a savoring strategy called “capitalization,” or sharing positive news with others, enhances our life satisfaction and increases our daily happiness, over and above the impact of the positive event itself. When mindfulness becomes a habit, the neural pathways in our brains linked to positive feelings become stronger and more active, increasing the chances we will continue to feel happier in the future. Now that you’ve built up your savoring skills, we think you’re ready to put your mindfulness and capitalization habits to work in an all-day experience. Yes, it will be more intense, and your savoring moments will be more varied—but you’re ready for it! |
| S-09 | Each day this week, write down at least one unpleasant thing that happened to you, and think about why you think it happened. Come up with one explanation you’re fairly confident about, plus a few others. Once you have a list of at least 3 possibilities (the bigger, the better), read through each of them. Which seems most plausible to you? Which seems least plausible? What evidence do you have in favor of and against each possibility? When this is done, go back to your original explanation. How sure do you feel about it now? | One effective way to combat negative thoughts resulting from an unpleasant experience is to consciously challenge them by coming up with alternative explanations for what caused the event, an idea taken from cognitive therapy. Martin Seligman also coined the phrase “learned optimism” in his book of the same name, which states that people can view negative experiences as unlucky situations that are not personal in nature, and aren’t permanent. In a study by researchers at the University of Pennsylvania, college students at risk for depression were randomized into an 8-week workshop group and a control group. Those in the workshop group used cognitive-behavioral techniques to learn how to improve their explanatory style, which included identifying automatic negative thoughts and underlying beliefs, as well as replacing negative thoughts with more constructive interpretations, which reduced depressive and anxiety symptoms, and improved well-being. |
| S-10 | Take a 30-minute walk in a park—or anywhere with interesting scenery and activity. Walk slowly at first, with eyes on the ground. Notice the tactile | Being mindful requires you to be aware of physical sensations, perceptions, thoughts and imagery. Numerous studies have shown that mindfulness meditation, especially when practiced regularly, can improve general coping with |
| | sensations of walking: the feeling of your feet, the temperature, the way your body feels. Once you can walk at a normal pace while paying attention to sensory experiences, start noticing sounds— but try not to think too much! Once you can attend to tactile and auditory sensations, start looking around and noticing any activity around you. | stresses from everyday life, as well as under more extraordinary conditions. According to studies, meditation can also lower high blood pressure and lessen chronic pain, anxiety and depression. It alters regions of the brain associated with memory, self-awareness and compassion. The added benefit of doing a walking meditation outside (especially in green spaces) is that spending time in nature reduces stress and helps people feel energetic and more alive, according to scientists at the University of Rochester. A recent study using mobile EEG devices to monitor participants’ emotions during a walk also found that people were more likely to experience meditative-like brain waves, and exhibit less frustration if they were walking in a green space, compared to a bustling shopping street or a busy business area. |
| S-11 | Decide on something to savor with a large group of people—a community or neighborhood group, or all of your coworkers, for example. Perhaps you can organize a group wine tasting or a class trip to a garden. Instruct the group on how to savor the experience using what you’ve learned from practicing your social savoring skills—take photos, note all the tiny details, and discuss what you value about the event and your time together. | Now that you’ve savored small moments, you’re ready to savor longer experiences and events with others. In a study about savoring strategies, researchers found that capitalization, or sharing, enhances our well-being and increases our everyday happiness, over and above the impact of the positive event itself. So your savoring event may only last for a few hours, but its mood-boosting effects could last for days. The research also suggests that sharing positive experiences may allow individuals to perceive themselves positively in the eyes of others, hence boosting self-esteem and facilitating positive appraisals of one’s life. Plus, scientists know that people enjoy things more when they do it with others, and that spending quality time with friends is a highly effective mood-booster. |
| S-12 | Catch yourself when you’re in the middle of an unpleasant experience. It can be an emotional state (being stressed) or an activity (being at a frustrating meeting). Notice everything that’s happening to you internally: your physical sensations, thoughts, feelings. Don’t evaluate (for example, wondering whether you’re thinking rationally), just experience it. Afterwards, write about what happened to you. Look at each aspect of the experience and see if you can connect how your physical sensations, thoughts, and feelings interacted with each other. | Robert Epstein, PhD, says that we have the power to stop stress before it even starts. There’s only a tenuous relationship between stressors (the things that cause us to feel anxiety) and stress, or our response to them. In other words, we can proactively build up our resilience against anxiety. And one effective way to do that is by practicing mindfulness. Being mindful requires you to be aware of physical sensations, perceptions, thoughts and imagery—even when your brain would rather be worrying about the unpleasant situation itself. (And yes, it’s much harder to practice living in the moment when you’re stressed or angry than it is when you’re feeling at peace with the world!) This exercise is derived from Mindfulness-Based Stress Reduction techniques. Numerous studies show that mindfulness practice, especially when done regularly, can improve general coping with stresses from everyday life, as well as under more extraordinary conditions. |
| S-13 | Want to feel happier and more connected with the world? (Who wouldn’t?) Take 10 minutes to try a simple exercise called loving-kindness meditation, which can help you do just that. Sitting quietly and comfortably, breathe naturally and pay attention to your breath. Spend a few minutes thinking loving, compassionate thoughts about yourself. (Think something like: “May I be happy.”) Then focus your loving thoughts on to someone who is close to you, like your children. (“May she be happy.”) Next, transfer your thoughts to all people. (“May they be happy.”) If your mind wanders, don’t feel bad about it—it’s natural!—just gently bring your thoughts back to the exercise. | Research shows that the practice of loving-kindness meditation shifts people’s day-to-day emotions to be more positive, and in so doing, it also increases their overall health and wellbeing. Barbara Fredrickson, Ph.D., Kenan Distinguished Professor of Psychology and Neuroscience at the University of North Carolina at Chapel Hill has been investigating the various benefits of positive emotions for 25 years and has explored the specific impact of loving-kindness mediation for more than a decade. Experiments by Fredrickson and her team show that people who practice loving-kindness meditation show more self-acceptance and enjoy more positive connections with friends, family, and co-workers. They also become more mindful, find more purpose in life, and are better able to savor the good. They even report fewer illness symptoms and show improved cardiac function. |
| T-01 | Write down everything that’s happened in the past week that you are grateful for—from friends to experiences to personal accomplishments. | Dr. Robert Emmons of the University of California-Davis has been at the forefront of research into gratitude interventions. His research shows that people who kept gratitude journals on a weekly basis exercised more regularly, reported fewer physical symptoms, felt better about their lives, and were more optimistic about the upcoming week compared to those who recorded hassles or neutral life events. He also found another benefit when it comes to attaining goals: Participants who kept gratitude lists were more likely to have made progress toward important personal goals (academic, interpersonal and health-based) over a two-month period compared to subjects in the other experimental conditions. |
| T-02 | Keep a gratitude journal about someone close to you. Write down everything they do that you’re grateful for, then review it at the end of the week. Are there any patterns in what this person tends to do that you’re grateful for? What generalizations can you draw about who this person is and what they mean to you? | One theory as to why gratitude could be linked with improved well-being is that grateful people have stronger coping strategies than people who aren’t grateful. In one study, gratitude was shown to relate to three broad categories of coping. First, grateful people are more likely to seek out support from their social networks when the need arises. Second, grateful people face life’s challenges head-on. They’re able to put their problems in perspective, plan solutions and learn from the experience. And third, grateful people are less likely to run from or deny their problems exist. These results may explain why grateful people are also less stressed. Keeping a gratitude log can also fortify your existing relationships by producing feelings of greater |
| | | connectedness. When you value someone, you tend to treat them better, which makes them feel good and treat you better. In several studies, researchers have found that people who are grateful towards particular individuals in their lives experience closer and “higher-quality” relationships with them, even if their gratitude is never directly expressed. |
| T-03 | If you created gratitude log about someone close to you during previous activity, show it to the person you wrote about. You can email it, go through your list on the phone, or better yet—find an opportunity to read your list out loud, in person. If you haven’t started log, begin by choosing someone in your inner circle and writing down everything they do that you’re grateful for over one-week period. Afterwards, review what you’ve written and look for any patterns. What generalizations can you draw about who this person is and what they mean to you? Finally, deliver your log to the person you wrote about. If you enjoyed this exercise, you can make this regular practice, delivering your “gratitude report” periodically to different people in your life. | In one study, participants were asked to write letter of gratitude to someone they wished to thank before reading the letter aloud to the recipient. After the experiment, they reported immediate increases in happiness and decreases in depressive symptoms, giving them short-term boost in well-being. In variant on the above experiment, scientists recommend keeping gratitude log for fixed period of time (for example, one month) about person in your life for whom you’re thankful. This exercise lets the giver experience the powerful happiness effects of delivering the gratitude report, but keeps the exercise fresh, since it can be replicated with different recipients each time. |
| T-04 | Jot down three things that happened today or yesterday that made you feel grateful. It could be something someone did for you, something you did for yourself, or just the simple fact that the sun was shining. Add photo, too! | In study conducted by Drs. Martin Seligman, Tracy Steen and Christopher Peterson, group of people was asked to practice this gratitude exercise every day for one week. Even though the exercise lasted just one week, at the one-month follow-up, participants were happier and less depressed than they had been at baseline, and they stayed that way at the three- and six-month follow-ups. This practice primes our mind for gratitude, and helps overcome the brain’s natural “negativity bias,” phenomenon by which we are wired to give more weight to negative rather than positive experiences or other kinds of information. |
| T-05 | Maybe you quit smoking or joined gym. Maybe you controlled your anger when you were provoked. Maybe you made someone laugh right when he/she needed it most. This isn’t the time for humility. What abilities come easily to you? Which of | Studies show that affirming our sense of personal worth can improve our ability to cope with stress. Affirmations get us to focus on higher values, rather than immediate impulses, and that boosts our ability to act in our best interests. In fact, even brief, daily affirmations can have long-term benefits if they break our cycle of negative rumination. So |
| | your talents do others admire? Ask close friend, family member or trusted co-worker which of your skills they find exceptional. Take some time to indulge in being grateful for who you are, whenever! | take few minutes and think of some things that are great about you! Studies show that affirming our sense of personal worth can improve our ability to cope with stress. Affirmations get us to focus on higher values, rather than immediate impulses, and that boosts our ability to act in our best interests. In fact, even brief, daily affirmations can have long-term benefits if they break our cycle of negative rumination. So take few minutes and think of some things that are great about you! |
| T-06 | Think of someone in your life whom you’ve never properly thanked. Your gratitude could be for particular instance when they helped you, or it could be very general—or both. It can be someone you see all the time, or someone you haven’t seen in while. Now, write letter detailing your gratitude to that person. It should be at least one page, ideally longer. Reflect, with great detail, on why you feel thankful towards them. | Numerous studies show that gratitude is one of the strongest predictors of positive mental health. People who are grateful are more satisfied with their relationships with friends and family. They’re happier, less depressed and less stressed. They feel more in control of their lives, have higher self-esteem and cope better with stress. In one study, ∼- participants were asked to write letter of gratitude to someone they wished to thank before reading the letter aloud to the recipient. After the experiment, they reported immediate increases in happiness and decreases in depressive symptoms. And research by Sonja Lyubomirsky, Rene Dickerhoof, and Julia Boehm has shown that you can get substantial boosts in happiness from writing gratitude note even if you don’t deliver your letter. |
| T-07 | If you wrote gratitude letter to someone you’re grateful to during an earlier activity, deliver it to them— ideally in person, but you could read it to them over the phone or send an email if the recipient is halfway across the globe. (Haven’t written gratitude letter yet? First think of someone in your life you’ve never properly thanked, then express your gratitude in note.) If you can, capture the note—or the moment itself!-in photo. | Numerous studies show that gratitude is one of the strongest predictors of positive mental health. People who are grateful are more satisfied with their relationships with friends and family. They’re happier, less depressed and less stressed. They feel more in control of their lives, have higher self-esteem and cope better with stress. In one study, participants were asked to write letter of gratitude to someone they wished to thank before reading the letter aloud to the recipient. After the experiment, they reported immediate increases in happiness and decreases in depressive symptoms. |
| T-08 | Think of something or someone in your life for whom you’re grateful. Discuss your feelings with someone else who shares that same gratitude. It may help if you have written gratitude letter about the target of the discussion, so you can share it with | Numerous studies show that gratitude is one of the strongest predictors of positive mental health. People who are grateful are more satisfied with their relationships with friends and family. They’re happier, less depressed and less stressed. They feel more in control of their lives, have higher self-esteem and cope better with stress. |
| | the other person to spark the discussion. | This exercise is variant of several interventions that involve either keeping gratitude log or writing gratitude letter. Both Sonja Lyubormirsky and Martin Seligman have conducted studies with participants asked to write letter of gratitude to someone they wished to thank (and in some cases, delivering it to that person). In both cases, they reported immediate increases in happiness and decreases in depressive symptoms. Scientists also know that people enjoy things more when they do it with other people, and that spending quality time with others is highly effective mood-booster. What could be more powerful than bonding with friend over your shared gratitude? |
| T-09 | Think of something, great or small, that you feel grateful for and describe it in few words. Add photo too! | Numerous studies show that gratitude is one of the strongest predictors of positive mental health. People who are grateful are more satisfied with their relationships with friends and family. They’re happier, less depressed and less stressed. They feel more in control of their lives, have higher self-esteem and cope better with stress. When the going gets tough, grateful people learn from the experience. They don’t avoid the problem, deny that anything’s wrong, or blame themselves. That’s probably why grateful people also sleep better! In 2005 study, some participants were asked to transcribe, every night for one week, three positive events that happened during their day, as well as the causes of these events. Compared to participants in the control group, those who reflected on three positive events experienced more happiness. |

### Specific to Migraine Digital Therapeutic

The first intervention in the migraine digital therapeutic provides information about symptoms and side effects, MDD and/or GAD symptoms, and how those symptoms are related to the treatment the patient needs and will complete. The therapeutic modality label for this type of intervention is psychoeducation. Later in treatment, an intervention that provides information about how specific therapeutic activities (mindfulness, for example) help GAD and/or MDD may be included. It also falls under psychoeducation.

When personalization of the treatment for key interest areas or disease-specific therapy is sought, it is important that the therapeutic modality remains the same. One cognitive intervention may be replaced with another cognitive intervention, but replacing cognitive intervention with psychoeducation intervention would be avoided. The complete order of therapeutic modalities is referred to as the indication treatment sequence. It is the order of all interventions over the multi-week treatment that is an important consideration in designing any digital therapeutic for maximum efficacy and adherence levels. Following sequence also impacts the treatment’s safety and efficacy, these factors have been considered in designing the multi-week treatment.

In treatment relevant to MDD and/or GAD, targeting and preventing worry behaviors such as frequent telephone calls to loved ones, refusal to read obituaries, or cleaning one’s house daily in case someone drops by, may be monitored. The therapy helps the patient focus on the specific behavior that is relevant to that patient. Flexibility is also essential in therapy when patient does not enjoy or cannot complete specific intervention. For example, progressive muscle relaxation is standard intervention in many MDD and/or GAD treatments. Briefly, the patient tenses then relax muscles throughout their body to achieve more relaxed physiological state. However, some patients experience paradoxical “relaxation-induced anxiety” that predicts poor outcomes. For patients who experience such anxiety, it is appropriate to find an alternative method for providing relaxation.

It is imperative to personalize CBT-based treatments for people suffering from chronic medical conditions like migraine. While the active ingredients are the same for people living with vs. without these conditions, some language, examples, and recommendations can provoke negative reactions in people with chronic medical conditions. People who require the use of walker or wheelchair, for example, might not appreciate walking meditation. People who have recently had an organ transplant may be unable to travel far from their medical support team, so examples involving travel might need to be reworked. These kinds of changes do not fundamentally alter the treatment but provide more supportive treatment experience. It is intended to use migraine digital therapeutic to provide personalized treatment to specific populations in which MDD and/or GAD is prevalent, such as people diagnosed with migraine.

In migraine sufferers and especially persons suffering chronic migraine, untreated anxiety and depression can contribute to poor control over the condition and exacerbate physical symptoms. Treating anxiety and depression in people living with migraine may or may not directly impact their physical health, but by reducing negative emotions and unhelpful behavioral patterns, patients can manage their conditions more effectively. Management of the condition also includes management of any side effects resulting from medications the patient is taking to treat their condition. For example, a CGRP receptor antagonist may have intestinal side-effects that can be addressed and specifically managed with particular activities and tracks.

CBT and related behavioral therapies can reduce anxiety and depression in people with chronic medical conditions. However, some personalization is required to ensure the treatment resonates with the patients. Such personalization is not consistently achievable in traditional, face to face therapy. Economics, logistics, training and organization are merely the most readily apparent reasons for this. Given the number of different factors involved per different chronic condition, it is simply not possible to match properly trained therapists with each patient suffering chronic condition. Clinicians with advanced training in CBT and other important therapies would need to complete supplemental training in the specific population to ensure they are fully prepared to provide such treatment at optimum levels. It is simply not possible for given clinician to have training across even significant percentage of all chronic conditions, symptomologies, side effect profiles and other potential therapeutic areas. A digital therapeutic is, in contrast, ideally suited to provide therapy directed to essentially any number of conditions, symptoms, side effects, etc.; therapy not only highly personalized to an individual patient but also personalized to one or more conditions impacting the mental health of the individual patient. The above-mentioned optimum level of treatment based upon proper training, ready access to all tools updated to the time of treatment, integration of all available research/trials, selecting appropriate protocols, etc., is not possible for even the best human therapist. For digital therapeutic, however, such personalized treatment is possibility. The ability to scale digital therapeutics is even more important for those living with chronic medical conditions than those who are not.

The present migraine digital therapeutic provides standardized series of interventions. The product may offer personalized experience based on the patient’s key area-of-interest (AOI) as an overlay on the standardized activity series. Some examples of AOIs are family, career, and physical wellness. The patient can choose an AOI or complete the treatment without AOI personalization. The different options may have the same indication treatment sequence or modified one. The therapeutic modality of each intervention will be the same for each of the treatment options or may be different. For identical therapeutic modalities it is expected that the efficacy of each option is likely to be identical.

Table 3 below shows an example of three activities from the migraine digital therapeutic. Based on the Indication Treatment Sequence created for the product, the first three interventions may be selected from the following therapeutic modalities: Mindfulness, Cognitive, Acceptance. The Career option’s specific interventions are focus on your breath for minutes, reframe negative thought--career focus, and identify fact that is hard for you to accept--career focus. The family option’s specific interventions are Focus on your breath for minutes, reframe negative thought--family focus, radical acceptance--family focus. The same principle applies across the full migraine digital therapeutic indication treatment sequence.

TABLE 3

| | | | |
|---|---|---|---|
| Intervention | Area of Interest: Career | Area of Interest: Family | Therapeutic Modality |
| | Focus on your breath for minutes | Focus on your breath for minutes | MINDFULNESS |
| | Choose negative thought related to your career. Put the thought on trial. Imagine you are lawyer. What is the evidence for and against this thought? | Choose negative thought related to your family. Put the thought on trial. Imagine you are lawyer. What is the evidence for and against this thought? | COGNITIVE |
| | Identify something about your job that you wish were different. Describe how it is right now in detail. Try to identify what makes it hard for you to accept that this is true about your job. | Think of something about one member of your family that really drives you crazy. Now take deep breath and focus on letting go of your efforts to change this. Work toward accepting at deep level that this person is not going to change this part of who they are. Think about how your own actions can change if you give up the idea that this person will change. | ACCEPTANCE |

Personalizing treatment for people living with chronic medical conditions like migraine can fill significant gap in mental health treatment as well as in the treatment of concomitant migraine symptoms or treatment side effects. As stated previously, these treatments are effective but require additional knowledge and training in order to be delivered efficiently, properly and competently. It is unrealistic to expect busy practitioners to gain needed expertise in each subgroup of people living with chronic medical conditions. Therefore, it is particularly valuable to have digital therapeutic that can be personalized and delivered efficiently at scale to everyone who needs it.

The methods for personalizing migraine digital therapeutic for people living with chronic medical conditions are similar to the methods for adapting it to an area of interest. Changes may be made to ensure the interventions are appropriate and impactful for given subpopulation, changes to the indication treatment sequence may be assessed but may be unnecessary. The interventions included in versions of migraine digital therapeutic that have been personalized for people living with chronic medical conditions will have interventions that reflect the same modalities that may be provided in the same or different order as in the standard version.

Table 4 below shows the three options for personalization for an intervention. The need to change interventions are expected to be highly variable depending upon the condition, symptoms, side effects and related concerns that will be fact dependent from condition to condition. Some minimal changes are shown in examples below. These include modifications to wording, such as different example to illustrate an idea, or physical exercise modification to allow for common physical limitation. A small number of interventions may be replaced with different intervention from the same therapeutic modality. Example below illustrates how one intervention from the “mindfulness” modality is exchanged for another. By adding these subtle personalization elements, more supportive treatment may be developed that will encourage people with specific chronic conditions to engage with the treatment more than they would without such personalization.

Table 4 shows interventions that may appear in standard MDD and/or GAD treatment compared to migraine-oriented model. The wording has been modified to make it appropriate for someone with migraine. Intervention is identical for the two treatment models. Intervention has very small change, but is nearly identical between the two treatment models. Intervention is an entirely different activity for the standard MDD and/or GAD treatment model vs the migraine-oriented MDD and/or GAD treatment model, but it comes from the same therapeutic or behavioral modality.

TABLE 4

| | | | |
|---|---|---|---|
| Example | No chronic condition | Chronic condition: Migraine | Therapeutic Modality |
| (no change) | Keep daily diary of your worry content, anxiety level, and whether it’s current or potential problem. | Keep daily diary of your worry content, anxiety level, and whether it’s current or potential problem. | DAILY MONITORING |
| (minimal change) | Worry is part of life for everyone, but for people with GAD it can get out of control. The physiological arousal that comes with excessive worry can make it hard to concentrate, interfere with your sleep, and make you irritable. | Worry is part of life for everyone, but for people with GAD it can get out of control. The physiological arousal that comes with excessive worry can make it hard to concentrate, interfere with your sleep, and make you irritable. For people with migraine, these problems can make it harder to | PSYCHOEDUCATION |
| | | follow your treatment plan and stay healthy. | |
| (different intervention, but same therapeutic modality) | One good way to manage worry is to refocus your attention from worry about the future to vigorous activity, such as running or jumping jacks. Choose level that is difficult for you, but that you are confident you can complete. If you have heart rate monitor, see if you can get your heart rate up to somewhere between 85 and 145 bpm for about minutes. Slowly cool down and see how you feel. | One healthy way to manage worry is to engage in something enjoyable instead. This will help get you out of your negative thought spiral and focused on the present. Choose something you enjoy, like cooking, playing musical instrument, or doing crossword puzzle. Try to throw yourself into it completely and take break from worry. | MINDFULNESS |

Modifying a generally useful and potentially FDA cleared product for a specific patient population has several benefits. There are often small changes that need to be made to ensure that the more specific population’s developed skill sets resonate within the app.

An example of small changes for migraine is that people with migraines, especially toward more severe end of spectrum, might worry about the onset of their next migraine. While many worrying thoughts are unlikely to come true, the question of when a migraine will occur in a person with chronic migraine is both valid and reasonable. The therapeutic product could address this and encourage the patient to focus instead on considering how they will cope when the migraine arrives.

It is very useful to include additional information helpful to people with migraine, such information may be integrated with various portions of the app. For example, some conditions are treated with reasonable number of medication types or have particular symptoms. Further, side-effects of medication types are also a known issue that may be planned for in the app. Migraine is typically dealt with using one or more of about 6-8 medications or other treatments of variable efficacy, patient to patient. The efficacy of these medications also varies from symptom to symptom. The chatbot integrated with the app, e.g., Anna, might therefore ask about these medications, symptoms and side-effects and reference them in future dialogues.

Another potentially useful feature is to connect patients suffering the same condition to each other through our product’s community features. Such feature may even drill down to particular symptoms and side-effects impacting a group of patients and connect them with reference thereto. These different groups might require different guidelines for discussions. For example, people with migraine who are recovering from surgery might want a community where details of triggers, aura, etc. are discussed or, potentially, not discussed. Similarly, a community discussing the efficacy of treatments might be interesting to a patient prior to deciding on whether to try that particular treatment.

A migraine digital therapeutic app may be used in the home as prescription device, under the management of licensed healthcare provider, for the treatment of migraine. The migraine app presented here has been developed under design controls developed as part of the Happify’s Quality System.

The development of the migraine digital therapeutic app has operated under applicable FDA regulations, FDA Guidance and consensus standards for software as medical device. This includes conformance with the following: 21 CFR 820.30 Design Controls, Guidance for the Content of Premarket Submissions for Software Contained in Medical Devices, Content of Premarket Submissions for Management of Cybersecurity in Medical Devices, Software as Medical Device (SaMD): Clinical Evaluation, ISO 14971, and IEC 62304. Also, each release of the device undergoes software testing in accordance with FDA’s Guidance, “General Principles of Software Validation” to further ensure that the software performs as intended.

Key design and development elements of a quality system include configuration management plan, software requirements specification, software development plan, software verification & validation plan, software risk analysis, and software defect tracking. Functional verification testing ensure the software performs per specification prior to clinical validation. Development through the design control and risk management processes provide the results and data necessary to demonstrate safety, effectiveness and overall quality of the migraine digital therapeutic app. Iterative bench testing and bug fixes are captured and documented by the developers. More formal verification and validation testing will be conducted subsequent to release. Verification and validation tests provide traceability back to design documents and the IEC 62304 requirements. In addition to software testing, the developer conducts reliability testing and human factors testing. Usability and risk of user error (intended and unintended miscue) may be studied though human factors engineering studies. Bugs and defects identified at this stage are captured using a tracking system. During the validation stage fixes would be approved in formal change order (CO) protocols.

### Additional Embodiments

The foregoing disclosure of a digital therapeutic app for the treatment of migraine is not intended to be limiting.

A component of the invention lies in acquiring ongoing and real time input data from the user and performing analysis to respond more empathetically and more emotionally and more in context. However, the extent of the analytic capability by the AI is not limited to simply detecting the “tone” or identifying certain “topics.” For example, the artificially intelligent computing system can analyze input data to ascertain whether the user is answering the question truthfully, whether the user is only providing a partial answer to an inquiry, whether the user is engaged with enthusiasm or lack of enthusiasm, the extent to which the user is interested in the activity being performed, and whether the user prefers certain types of activities over other types of activities. In addition, when the user’s response is analyzed, the computing system may detect not only topics, but also entities, and what the user’s sentiment is toward these entities. Any of these analyses may be performed in addition to, or in conjunction with, the above-described analyses to develop a conversation that is emotionally specific.

In accordance with the present invention, the techniques as disclosed herein for the computing system to utilize AI in demonstrating empathy and providing more in context response goes far beyond merely automating what may occur in a typical current-day therapy session. One most notable advantage of the present computing system is its capability of providing a “super human” therapy or coaching session. A human therapist/coach bases his or her treatment based on familiarity with X number of patients. In contrast, the computing system of the present invention implements mirroring and other data-driven methods based on data collected from millions of users. For example, the computing system of the present invention knows how people tend to respond to a certain question much better than any single human therapist. Moreover, the computing system in accordance with the present invention can choose from a very large number of prompts, or generate new prompts from using natural language generation tools, some of which may include scientific facts, quotes, etc. in a way that significantly exceeds the capacity of a single human therapist. For example, if a user is into Indonesian movies from the 1950 s, the computing system can find and/or generate a prompt weaving that into the conversation. No human therapist can personally relate to all topics that interest millions of people.

In accordance with the present invention, the English language is not intended to limit application or scope of any of the foregoing aspects of the present invention. For example, the classifier may be trained in multiple languages and one or more of the known techniques employed may work equally in different languages. In some embodiments, the artificial intelligence of the computing system may also learn cultural uniqueness in regards to tone, or in regards to conveyance of empathy in general, and adapt accordingly.

As herein used, a computer readable storage medium is not to be construed as being transitory signals per se, such as radio waves or other freely propagating electromagnetic waves, electromagnetic waves propagating through a waveguide or other transmission media, or electrical signals transmitted through a wire. The computer readable storage medium may be, but is not limited to, e.g., a magnetic storage device, an electronic storage device, an optical storage device, a semiconductor storage device, an electromagnetic storage device, or any suitable combination of the foregoing, and can be a tangible device that can retain and store instructions for use by an instruction execution device. The following is a list of more specific examples of the computer readable storage medium, but is not exhaustive: punch-cards, raised structures in a groove, or other mechanically encoded device having instructions recorded thereon, an erasable programmable read-only memory, a static random access memory, a portable compact disc read-only memory, a digital versatile disk, a portable computer diskette, a hard disk, a random access memory, a read-only memory, a memory stick, a floppy disk, and any suitable combination of the foregoing.

The operations of the present invention may be carried out by program instructions which may be machine instructions, machine dependent instructions, microcode, assembler instructions, instruction-set-architecture instructions, firmware instructions, state-setting data, or either source code or object code written in any combination of one or more programming languages, including an object oriented programming language such as, but not limited to, C++, Python, Java, and other conventional procedural programming languages. The program instructions, while having the capability of being executed entirely on the computer of the user, may also be executed partly on the computer of the user, partly on a remote computer and partly on the computer of the user, entirely on the remote computer or server, or as a stand-alone software package. In the “entirely on the remote computer or server” scenario, the remote computer may be connected to the user’s computer through any type of network, including a wide area network or a local area network, or the connection may be made to an external computer. In some embodiments, electronic circuitry including, e.g., field-programmable gate arrays, programmable logic circuitry, or programmable logic arrays may execute the program instructions by utilizing state information of the program instructions to personalize the electronic circuitry, in order to perform aspects of the present invention.

These program instructions may be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. These program instructions may also be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The computer readable program instructions may also be loaded onto a computer, other programming apparatus, or other device to produce a computer implemented process, such that the instructions which execute on the computer, other programmable apparatus, or other device implement the functions/acts specified in the flowchart and/or block diagram block or blocks.

Aspects of the present invention are described herein with reference to block and/or other diagrams and/or flowchart illustrations of methods, apparatus, and computer program products according to the present invention’s embodiments. It will be understood that each block of the block and/or other diagrams and/or flowchart illustrations, and combinations of blocks in the block and/or other diagrams and/or flowchart illustrations, can be implemented by program instructions that are readable by a computer.

The block and/or other diagrams and/or flowchart illustrations in the Figures are illustrative of the functionality, architecture, and operation of possible implementations of systems, methods, and computer program products according to the present invention’s various embodiments. In this regard, each block in the block and/or other diagrams and/or flowchart illustrations may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently or sometimes in reverse order, depending upon the functionality involved. It will also be noted that each block of the block and/or other diagram and/or flowchart illustration, and combinations of blocks in the block and/or other diagram and/or flowchart illustration, can be implemented by special purpose hardware-based systems that perform the specified functions or acts or carry out combinations of special purpose hardware and computer instructions.

In view of the foregoing disclosure, an inventive computing system and technique for interacting with users have been described. In accordance with the disclosure provided herein, a computing system engages with users in a novel manner, for the purpose of improving levels of happiness, or more broadly, to alleviate or reduce symptoms of mental health conditions such as depression and anxiety, such interaction entailing simulation of human emotion and/or human cognitive skills by the computing system, to beneficially result in a high level of engagement by the users and better efficacy of the overall interaction, leading to higher increases in the behavior and/or the psychological well-being of the users. In further accordance with the disclosure provided herein, the computing system receives and analyzes on-going supply of user data for the purposes of identifying topics and tone of the user’s communication and responding with a mirroring or an appropriate tone that most empathetically advances an interactive session with the user. Finally, in accordance with the disclosures provided herein, the computing system proactively recognizes the user’s adherence or enthusiasm toward a given program and recommends alternative options that have been determined to better suit the user’s current physical and/or psychological states.

The present disclosure concerns implementing a prescription or non-prescription digital therapeutic configured to treat major depressive disorder (MDD), general anxiety disorder (GAD) and related mental health challenges. In particular, the disclosure concerns MDD, GAD, lower level depressive/anxiety disorders and related mental health conditions that occur in the context of patients suffering from migraine. Such conditions may be comorbidities of migraine, related to migraine symptoms or related to side-effects from migraine treatment(s). The digital therapeutic may include cognitive behavioral therapy (CBT) or other cognitive therapy as well as behavioral activation. Administration of CBT may serve to correct distorted cognitions that can cause patients to have a negative view of themselves, the world, their current and future context.

The digital therapeutic may include a number of interfaces of various types to help a user understand automatic thoughts, common situations and symptoms related to negative aspects of their mental health. The user may also check their thoughts against a set of common cognitive distortions or “thinking traps” and identify alternative cognitions that may prove helpful. The user may be exposed to ‘known’ automatic and alternative thoughts collected from a sample of people, sometimes a large sample of people, with similar circumstances to the user.

What is claimed is:

1. A treatment protocol comprising:
an abortive drug configured to inhibit at least one of a plurality of a calcitonin superfamily of peptides, the abortive drug adapted to bind with a plurality of calcitonin gene-related peptides (CGRP) receptors, the abortive drug further comprising an abortive dosage,
wherein the plurality of the calcitonin superfamily of peptides comprises one or more CGRPs,
wherein the one or more CGRPs comprise a 37 amino acid vasoactive neuropeptide,
wherein the abortive drug bound to the plurality of CGRP receptors mitigates vasodilation; and
a client device comprising at least one processor, at least one memory comprising a drug database and computer-executable instructions which, when executed by the at least one processor, cause the client device to:
receive, via the at least one memory, an initial abortive drug regimen,
wherein the abortive drug regimen comprises one or more abortive drugs and one or more abortive dosages; and
receive, via the client device, a user input through an interactive session,
wherein the interactive session is configured for bidirectional communication between the client device and a user.

2. The treatment protocol of claim 1, the computer-executable instructions which, when executed by the at least one processor, further cause the client device to:
extract, via the at least one processor, medication-relevant information from the user input,
wherein the medication-relevant information is correlated to drug-specific information stored within the drug database; and
determine, via the at least one processor, based on the medication-relevant information and the user input, a migraine severity of the user.

3. The treatment protocol of claim 2, the computer-executable instructions which, when executed by the at least one processor, further cause the client device to:
display, via the client device, an alert configured to inform the user to administer the one or more abortive dosages if the migraine severity is greater than an abortive drug threshold; and
analyze, via the at least one processor, the medication-relevant information in view of the drug-specific information to determine whether the initial abortive drug regimen is being adhered to and whether the medication-relevant information comprises one or more side effects or one or more positive effects,
wherein the one or more side effects and the one or more positive effects are correlated to the one or more abortive drugs via the drug database.

4. The treatment protocol of claim 3, further comprising:
a preventative drug configured to inhibit at least one of the plurality of the calcitonin superfamily of peptides, the preventative drug adapted to bind with the plurality of CGRP receptors, the preventative drug further comprising a preventative dosage,
wherein the preventative drug bound to the plurality of CGRP receptors mitigates vasodilation; and
the computer-executable instructions which, when executed by the at least one processor, further cause the client device to:
receive, via the at least one memory, an initial preventative drug regimen,
wherein the preventative drug regimen comprises one or more preventative drugs and one or more preventative dosages; and
display, via the client device, a reminder configured to inform the user to administer the one or more preventative dosages according to the initial preventative drug regimen.

5. The treatment protocol of claim 4, the computer-executable instructions which, when executed by the at least one processor, further cause the client device to:
analyze, via the at least one processor, the medication-relevant information in view of the drug-specific information to determine whether the initial preventative drug regimen is being adhered to and whether the medication-relevant information comprises one or more side effects or one or more positive effects,
wherein the one or more side effects and the one or more positive effects are correlated to the one or more preventative drugs via the drug database.

6. The treatment protocol of claim 5, the computer-executable instructions which, when executed by the at least one processor, further cause the client device to:
generate, via the at least one processor, an activity within the interactive session, the activity configured to increase the one or more positive effects and decrease the one or more side effects,
wherein the activity is one of a plurality of activity types, and
wherein the activity comprises an activity content;
receive, via the at least one memory, an activity response; and
analyze, via the at least one processor, the activity response to determine whether the one or more side effects and the one or more positive effects have fluctuated relative to the previously assessed one or more side effects and one or more positive effects.

7. The treatment protocol of claim 6, the computer-executable instructions which, when executed by the at least one processor, further cause the client device to:
determine, via the at least one processor, an activity content efficacy and an activity type efficacy; and
generate, via the at least one processor, a succeeding activity,
wherein the succeeding activity is one of the plurality of activity types according to the activity type efficacy, and
wherein the succeeding activity comprises the activity content according to the activity content efficacy.

8. The treatment protocol of claim 7, further comprising:
one or more sensors in electrical communication with the client device, the one or more sensors configured to capture the user input and the activity response, and transmit said user input and said activity response to the at least one memory.

9. The treatment protocol of claim 7, the computer-executable instructions which, when executed by the at least one processor, further cause the client device to:
generate, via the at least one processor, an updated abortive drug regimen based on the initial abortive drug regimen, the one or more side effects, and the one or more positive effects.

10. The treatment protocol of claim 9, the computer-executable instructions which, when executed by the at least one processor, further cause the client device to:
generate, via the at least one processor, an updated preventative drug regimen based on the initial preventative drug regimen, the one or more side effects, and the one or more positive effects.
